SEQUENCE LISTING (72 sequences)

SEQ ID NO: 1 - protein, 590 aa, synthetic construct
  REGION 1..590: note = addressing polypeptide
MNKAYSIIWSHSRQAWIVASELARGHGFVLAKNTLLVLAVVSTIGNAFAVDHHHHHHLEALFQGPGTQKQRTELENLYFQGEQKLISEEDLSRVNNNGSIVINNSIINGNITNDADLSFGTAKLLSATVNGSLVNNKNIILNPTKESAAAIGNTLTVSNYTGTPGSVISLGGVLEGDNSLTDRLVVKGNTSGQSDIVYVNEDGSGGQTRDGINIISVEGNSDAEFSLKNRVVAGAYDYTLQKGNESGTDNKGWYLTSHLPTSDTRQYRPENGSYATNMALANSLFLMDLNERKQFRAMSDNTQPESASVWMKITGGISSGKLNDGQNKTTTNQFINQLGGDIYKFHAEQLGDFTLGIMGGYANAKGKTINYTSNKAARNTLDGYSVGVYGTWYQNGENATGLFAETWMQYNWFNASVKGDGLEEEKYNLNGLTASAGGGYNLNVHTWTSPEGITGEFWLQPHLQAVWMGVTPDTHQEDNGTVVQGAGKNNIQTKAGIRASWKVKSTLDKDTGRRFRPYIEANWIHNTHEFGVKMSDDSQLLSGSRNQGEIKTGIEGVITQNLSVNGGVAYQAGGHGSNAISGALGIKYSF

SEQ ID NO: 2 - protein, 441 aa, synthetic construct
  REGION 1..441: note = hydrophilic domain of cytochrome P450
HRNLTDLAKRFGEILLLRMGQRNLVVVSSPELAKEVLHTQGVEFGSRTRNVVFDIFTGKGQDMVFTVYGEHWRKMRRIMTVPFFTNKVVQQYRYGWEAEAAAVVDDVKKNPAAATEGIVIRRRLQLMMYNNMFRIMFDRRFESEDDPLFLKLKALNGERSRLAQSFEYNYGDFIPILRPFLRNYLKLCKEVKDKRIQLFKDYFVDERKKIGSTKKMDNNQLKCAIDHILEAKEKGEINEDNVLYIVENINVAAIETTLWSIEWGIAELVNHPEIQAKLRHELDTKLGPGVQITEPDVQNLPYLQAVVKETLRLRMAIPLLVPHMNLHDAKLGGFDIPAESKILVNAWWLANNPDQWKKPEEFRPERFLEEEAKVEANGNDFRYLPFGVGRRSCPGIILALPILGITIGRLVQNFELLPPPGQSKIDTDEKGGQFSLHILKH

SEQ ID NO: 3 - protein, 468 aa, synthetic construct
  REGION 1..468: note = hydrophilic domain of a plant cytochrome P450
IPVPIFGNWLQVGDDLNHRNLTDLAKRFGEILLLRMGQRNLVVVSSPELAKEVLHTQGVEFGSRTRNVVFDIFTGKGQDMVFTVYGEHWRKMRRIMTVPFFTNKVVQQYRYGWEAEAAAVVDDVKKNPAAATEGIVIRRRLQLMMYNNMFRIMFDRRFESEDDPLFLKLKALNGERSRLAQSFEYNYGDFIPILRPFLRNYLKLCKEVKDKRIQLFKDYFVDERKKIGSTKKMDNNQLKCAIDHILEAKEKGEINEDNVLYIVENINVAAIETTLWSIEWGIAELVNHPEIQAKLRHELDTKLGPGVQITEPDVQNLPYLQAVVKETLRLRMAIPLLVPHMNLHDAKLGGFDIPAESKILVNAWWLANNPDQWKKPEEFRPERFLEEEAKVEANGNDFRYLPFGVGRRSCPGIILALPILGITIGRLVQNFELLPPPGQSKIDTDEKGGQFSLHILKHSTIVAKPRSF

SEQ ID NO: 4 - protein, 395 aa, synthetic construct
  REGION 1..395: note = hydrophilic domain of a plant cytochrome P450
MDIFTSLLYLALILFFSLQVFRSFAFPKHKRLPPGPKPRPIIGSLLELGDQPHRSLARLSESYGPFMHLKLGQVTTVVISSTTMAKEVLQANSQVVSSRTITDASRAHRHSDFSMVMLPVSPLWRNLRKISNSHLLSSKALDGNMELRNKKVQELLNDVHKSVQAGEAVEIASLSFRATLNLLSTTFFSMDMADDTNSVTLKELKEAMSHMMEELGKPNLADYFPFLQKIDPQGIRRRNTVTFRKLINLFGRIIDQRLKVREASGSLKDDDILDTLINMMVVDQEKKEDQLDKTIIEHFLLDLFSAGTETTSTTLEWAMAELVKAPEIMSKARAELDQVIGKGNQVKESDVSRLPYLQAIVKETFRMHPTAPLLIPRKADSDIEISDYIIPKDAQ

SEQ ID NO: 5 - protein, 359 aa, synthetic construct
  REGION 1..359: note = hydrophilic domain of a plant cytochrome P450
KPRPIIGSLLELGDQPHRSLARLSESYGPFMHLKLGQVTTVVISSTTMAKEVLQANSQVVSSRTITDASRAHRHSDFSMVMLPVSPLWRNLRKISNSHLLSSKALDGNMELRNKKVQELLNDVHKSVQAGEAVEIASLSFRATLNLLSTTFFSMDMADDTNSVTLKELKEAMSHMMEELGKPNLADYFPFLQKIDPQGIRRRNTVTFRKLINLFGRIIDQRLKVREASGSLKDDDILDTLINMMVVDQEKKEDQLDKTIIEHFLLDLFSAGTETTSTTLEWAMAELVKAPEIMSKARAELDQVIGKGNQVKESDVSRLPYLQAIVKETFRMHPTAPLLIPRKADSDIEISDYIIPKDAQ

SEQ ID NO: 6 - protein, 51 aa, synthetic construct
  REGION 1..51: note = binding polypeptide
PGGSGGGSGGGGSGGGGSGGGGSGGGGSGGGGSGGGGSGGGGSGGGSGGSP

SEQ ID NO: 7 - protein, 610 aa, synthetic construct
  REGION 1..610: note = hydrophilic domain of NADPH P450 reductase from plant cytochrome P450
TRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 8 - protein, 563 aa, synthetic construct
  REGION 1..563: note = hydrophilic domain of NADPH P450 reductase from plant cytochrome P450
YEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSE

SEQ ID NO: 9 - DNA, 1608 bp, synthetic construct
  misc_feature 1..1608: note = polynucleotide encoding a fusion protein
  source 1..1608: mol_type = other DNA
accatgggcaataaggcctacagtatcatttggagccactccagacaggcctggattgtggcctcagagttagccagaggacatggttttgtccttgcaaaaaatacactgctggtattggcggttgtttccacaatcggaaatgcatttgcagtcgaccaccatcaccatcaccatctggaagcgctgttccagggtccgggtaccgctacagtgaatggtagtcttgttaataacaaaaatatcattcttaatcctacaaaagaaagtgcggccgctataggtaatactcttaccgtgtcaaattatactgggacaccgggaagtgttatttctcttggtggtgtgcttgaaggagataattcacttacggaccgtctggtggtgaaaggtaatacctctggtcaaagtgacatcgtttatgtcaatgaagatggcagtggtggtcagacgagagatggtattaatattatttctgtagagggaaattctgatgcagaattctctctgaagaaccgcgtagttgccggagcttatgattacacactgcagaaaggaaacgagagtgggacagataataagggatggtatttaaccagtcatcttcccacatctgatacccggcaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagctgaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttctgataatga

SEQ ID NO: 10 - DNA, 1779 bp, synthetic construct
  misc_feature 1..1779: note = polynucleotide encoding a fusion protein
  source 1..1779: mol_type = other DNA
atgaataaggcctacagtatcatttggagccactccagacaggcctggattgtggcctcagagttagccagaggacatggttttgtccttgcaaaaaatacactgctggtattggcggttgtttccacaatcggaaatgcatttgcagtcgaccaccatcaccatcaccatctggaagcgctgttccagggtccgggtacccagaaacagcgtaccgagctcgaaaacctgtacttccagggtgaacagaaactgattagcgaagaagatctgtctagagtgaataacaatggaagcattgtcattaataacagcattataaacgggaatattacgaatgatgctgacttaagttttggtacagcaaagctgctctctgctacagtgaatggtagtcttgttaataacaaaaatatcattcttaatcctacaaaagaaagtgcggccgctataggtaatactcttaccgtgtcaaattatactgggacaccgggaagtgttatttctcttggtggtgtgcttgaaggagataattcacttacggaccgtctggtggtgaaaggtaatacctctggtcaaagtgacatcgtttatgtcaatgaagatggcagtggtggtcagacgagagatggtattaatattatttctgtagagggaaattctgatgcagaattctctctgaagaaccgcgtagttgccggagcttatgattacacactgcagaaaggaaacgagagtgggacagataataagggatggtatttaaccagtcatcttcccacatctgatacccggcaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagctgaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttctgataatga

SEQ ID NO: 11 - DNA, 1413 bp, synthetic construct
  misc_feature 1..1413: note = polynucleotide encoding a polypeptide comprising a hydrophilic domain of a plant cytochrome P450
  source 1..1413: mol_type = other DNA
cctggcccaatcccggttccaattttcggcaactggctacaagttggcgatgatttgaaccaccggaacttaaccgatctggctaagaggtttggtgagatcttgctgctacgcatggggcagaggaatctggtagttgtgtcttcgcctgagcttgctaaagaggtgttgcatacacaaggagtggagtttggttcgagaacaaggaatgttgtgttcgatatttttactgggaagggtcaggatatggtgtttacggtttatggtgagcattggaggaagatgaggaggatcatgaccgtaccctttttcaccaacaaagttgttcagcaatacaggtatgggtgggaggctgaggccgcggcggttgtggacgatgtgaagaagaatccggctgcagcaactgaaggaatcgtgatccgaagacggttacaactcatgatgtataacaacatgttcagaatcatgttcgacagacgattcgaaagtgaagatgatcccttgtttttgaaactcaaggcgttgaacggtgagaggagtcgattggcgcagagctttgagtacaactatggcgatttcatccctattttgcggccgtttttgagaaattatttgaagttgtgcaaggaagttaaagataaaaggattcagctcttcaaggattacttcgttgacgaaaggaagaagattggaagcactaagaaaatggacaacaatcagttgaaatgtgccattgatcacattcttgaagctaaagagaagggtgagatcaatgaagacaatgttctttacattgttgaaaacatcaatgttgcagcaatcgagacaactctatggtcgatcgaatggggaattgcggagctagttaaccatcccgagatccaagccaaactcaggcacgagctcgacaccaagctcgggcccggtgtccagatcaccgagcccgacgtccaaaacctcccttacctccaagccgtggtcaaggaaaccctccgtctccgtatggcgatcccgcttctagtcccacacatgaacctccatgacgctaagctcggcgggtttgacatcccggccgaaagcaagatcttggtcaacgcgtggtggttagcaaacaaccccgaccaatggaagaaacccgaggagtttaggccagagaggtttttggaagaggaagcgaaggttgaggctaacgggaatgattttaggtacttgccgtttggagtcgggagaaggagttgccccgggattattcttgcattgccgatacttggtattacaatcgggcgtttggtgcagaatttcgagctgttgcctccaccgggacagtctaagatcgataccgatgagaagggtgggcagtttagtttgcatatcttgaagcactctactatcgtagctaaacctaggtcattt

SEQ ID NO: 12 - DNA, 1518 bp, synthetic construct
  misc_feature 1..1518: note = polynucleotide encoding a polypeptide comprising a hydrophilic domain of a plant cytochrome P450
  source 1..1518: mol_type = other DNA
atggacctcctcctcatagaaaaaaccctcgtcgccttattcgccgccattatcggcgcaatactaatctccaaactccgcggtaaaaaattcaagctcccacctggcccaatcccggttccaattttcggcaactggctacaagttggcgatgatttgaaccaccggaacttaaccgatctggctaagaggtttggtgagatcttgctgctacgcatggggcagaggaatctggtagttgtgtcttcgcctgagcttgctaaagaggtgttgcatacacaaggagtggagtttggttcgagaacaaggaatgttgtgttcgatatttttactgggaagggtcaggatatggtgtttacggtttatggtgagcattggaggaagatgaggaggatcatgaccgtaccctttttcaccaacaaagttgttcagcaatacaggtatgggtgggaggctgaggccgcggcggttgtggacgatgtgaagaagaatccggctgcagcaactgaaggaatcgtgatccgaagacggttacaactcatgatgtataacaacatgttcagaatcatgttcgacagacgattcgaaagtgaagatgatcccttgtttttgaaactcaaggcgttgaacggtgagaggagtcgattggcgcagagctttgagtacaactatggcgatttcatccctattttgcggccgtttttgagaaattatttgaagttgtgcaaggaagttaaagataaaaggattcagctcttcaaggattacttcgttgacgaaaggaagaagattggaagcactaagaaaatggacaacaatcagttgaaatgtgccattgatcacattcttgaagctaaagagaagggtgagatcaatgaagacaatgttctttacattgttgaaaacatcaatgttgcagcaatcgagacaactctatggtcgatcgaatggggaattgcggagctagttaaccatcccgagatccaagccaaactcaggcacgagctcgacaccaagctcgggcccggtgtccagatcaccgagcccgacgtccaaaacctcccttacctccaagccgtggtcaaggaaaccctccgtctccgtatggcgatcccgcttctagtcccacacatgaacctccatgacgctaagctcggcgggtttgacatcccggccgaaagcaagatcttggtcaacgcgtggtggttagcaaacaaccccgaccaatggaagaaacccgaggagtttaggccagagaggtttttggaagaggaagcgaaggttgaggctaacgggaatgattttaggtacttgccgtttggagtcgggagaaggagttgccccgggattattcttgcattgccgatacttggtattacaatcgggcgtttggtgcagaatttcgagctgttgcctccaccgggacagtctaagatcgataccgatgagaagggtgggcagtttagtttgcatatcttgaagcactctactatcgtagctaaacctaggtcattttaa

SEQ ID NO: 13 - DNA, 1515 bp, synthetic construct
  misc_feature 1..1515: note = polynucleotide encoding a polypeptide comprising a hydrophilic domain of a plant cytochrome P450
  source 1..1515: mol_type = other DNA
atggatattttcacctccttactgtatcttgctctcattcttttcttttctcttcaagtcttccgttcctttgcgtttcctaaacacaaaaggcttccacctggtccaaaacctcgtcccatcatcggaagcctcttggagctcggcgaccaaccccacaggtccttggccaggctttccgagtcttacggcccgtttatgcatttgaagctcggccaagtcacgacggttgtcatttcctccaccaccatggctaaagaagtcctccaggcaaacagccaagtcgtctccagccggacaatcaccgacgcaagccgcgcccacagacacagcgattttagcatggttatgttgcccgtatcccctctgtggcgaaaccttcggaaaataagcaactcacacttgctttcctccaaggctcttgatggcaacatggagctgagaaacaaaaaggtgcaagagctcctaaatgatgtccacaaaagcgtccaggccggggaggcggtggagatcgcgagcctttctttcagagctactctgaatctcttgtccaccacatttttctccatggacatggcggatgacacaaattccgtcactctaaaagagctcaaggaggctatgtcgcacatgatggaagagttggggaagcctaacttggccgattatttcccgtttctacaaaagattgacccccaaggcattaggcggcgcaacacggttactttccggaaactgatcaacttgtttgggcgtatcatcgaccaaagattgaaagtgagagaagcgagtggttctttgaaagatgatgatattttagacactcttatcaacatgatggtggtggatcaggagaagaaagaggatcagcttgacaaaaccataattgaacattttttactggatttattttcagcggggactgaaacgacttcaaccacgttggagtgggcaatggctgagctagtaaaagcgccagagattatgtcaaaagcccgagcagagctagatcaagttataggcaaaggaaaccaagtgaaggaatcggacgtatctcgactcccttacttacaagccattgttaaagaaaccttccgcatgcaccctacagctccattattgattcctcgcaaagccgacagtgacatcgaaatctccgactatatcatcccgaaggatgctcaggtgattgtcaatgtatgggccattggtagagactcaagcacatgggaaaatcccgacaagtttataccggagaggtttttggacatcgatatagatgtcggaggccgggattttaagctcattccgttcggtgctggtcggagaatatgtcccggattcccattggcgatgcgaatgttgcacttgatgttggggtctttgcttcactcgtttgattggaagttggaagatggggttagacctgatgctctaaacatggatgaaaagtttggcctcaccttgcaaatggctcagcctttgcgagctatccccgtgccgacaaagcattag

SEQ ID NO: 14 - DNA, 1395 bp, synthetic construct
  misc_feature 1..1395: note = polynucleotide encoding a polypeptide comprising the hydrophilic domain of a plant cytochrome P450
  source 1..1395: mol_type = other DNA
atcatcggaagcctcttggagctcggcgaccaaccccacaggtccttggccaggctttccgagtcttacggcccgtttatgcatttgaagctcggccaagtcacgacggttgtcatttcctccaccaccatggctaaagaagtcctccaggcaaacagccaagtcgtctccagccggacaatcaccgacgcaagccgcgcccacagacacagcgattttagcatggttatgttgcccgtatcccctctgtggcgaaaccttcggaaaataagcaactcacacttgctttcctccaaggctcttgatggcaacatggagctgagaaacaaaaaggtgcaagagctcctaaatgatgtccacaaaagcgtccaggccggggaggcggtggagatcgcgagcctttctttcagagctactctgaatctcttgtccaccacatttttctccatggacatggcggatgacacaaattccgtcactctaaaagagctcaaggaggctatgtcgcacatgatggaagagttggggaagcctaacttggccgattatttcccgtttctacaaaagattgacccccaaggcattaggcggcgcaacacggttactttccggaaactgatcaacttgtttgggcgtatcatcgaccaaagattgaaagtgagagaagcgagtggttctttgaaagatgatgatattttagacactcttatcaacatgatggtggtggatcaggagaagaaagaggatcagcttgacaaaaccataattgaacattttttactggatttattttcagcggggactgaaacgacttcaaccacgttggagtgggcaatggctgagctagtaaaagcgccagagattatgtcaaaagcccgagcagagctagatcaagttataggcaaaggaaaccaagtgaaggaatcggacgtatctcgactcccttacttacaagccattgttaaagaaaccttccgcatgcaccctacagctccattattgattcctcgcaaagccgacagtgacatcgaaatctccgactatatcatcccgaaggatgctcaggtgattgtcaatgtatgggccattggtagagactcaagcacatgggaaaatcccgacaagtttataccggagaggtttttggacatcgatatagatgtcggaggccgggattttaagctcattccgttcggtgctggtcggagaatatgtcccggattcccattggcgatgcgaatgttgcacttgatgttggggtctttgcttcactcgtttgattggaagttggaagatggggttagacctgatgctctaaacatggatgaaaagtttggcctcaccttgcaaatggctcagcctttgcgagctatccccgtgccgacaaagcattag

SEQ ID NO: 15 - DNA, 153 bp, synthetic construct
  misc_feature 1..153: note = polynucleotide encoding a binding polypeptide
  source 1..153: mol_type = other DNA
ccgggcggttctggtggcggtagcggcggtggcggttctggcggtggcggtagcggcggtggcggttctggcggtggcggtagcggcggtggcggttctggcggtggcggtagcggcggtggcggttctggtggcggtagcggcggttctccg

SEQ ID NO: 16 - DNA, 2079 bp, synthetic construct
  misc_feature 1..2079: note = polynucleotide encoding a polypeptide comprising the hydrophilic domain of a plant cytochrome P450 NADPH reductase
  source 1..2079: mol_type = other DNA
atgacttctgctttgtatgcttccgatttgtttaagcagctcaagtcaattatggggacagattcgttatccgacgatgttgtacttgtgattgcaacgacgtctttggcactagtagctggatttgtggtgttgttatggaagaaaacgacggcggatcggagcggggagctgaagcctttgatgatccctaagtctcttatggctaaggacgaggatgatgatttggatttgggatccgggaagactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctggtga

SEQ ID NO: 17 - DNA, 1830 bp, synthetic construct
  misc_feature 1..1830: note = polynucleotide encoding a polypeptide comprising the hydrophilic domain of a plant cytochrome P450 NADPH reductase
  source 1..1830: mol_type = other DNA
actagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctgg

SEQ ID NO: 18 - DNA, 1436 bp, synthetic construct
  misc_feature 1..1436: note = tetracycline resistance gene
  source 1..1436: mol_type = other DNA
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgt

SEQ ID NO: 19 - DNA, 17 bp, synthetic construct
  misc_feature 1..17: note = T7 RNA polymerase gene promoter sequence
  source 1..17: mol_type = other DNA
taatacgactcactata

SEQ ID NO: 20 - DNA, 47 bp, synthetic construct
  misc_feature 1..47: note = T7 RNA polymerase gene terminator sequence
  source 1..47: mol_type = other DNA
tagcataaccccttggggcctctaaacgggtcttgaggggttttttg

SEQ ID NO: 21 - DNA, 22 bp, synthetic construct
  misc_feature 1..22: note = primer LAEWXpr17-lacIQ
  source 1..22: mol_type = other DNA
tggcgacaccatcgaatggtgc

SEQ ID NO: 22 - DNA, 20 bp, synthetic construct
  misc_feature 1..20: note = primer LAEWXpr02 Reverse
  source 1..20: mol_type = other DNA
tttagcttccttagctcctg

SEQ ID NO: 23 - DNA, 30 bp, synthetic construct
  misc_feature 1..30: note = primer LAEWXpr30
  source 1..30: mol_type = other DNA
tcatcatcatgcctaatgagtgagaattcc

SEQ ID NO: 24 - DNA, 23 bp, synthetic construct
  misc_feature 1..23: note = primer LAEWXpr31
  source 1..23: mol_type = other DNA
ttggtgcgcaaactattaactgg

SEQ ID NO: 25 - DNA, 43 bp, synthetic construct
  misc_feature 1..43: note = primer LAEWXpr32-1-fr
  source 1..43: mol_type = other DNA
ctcattaggcatgatgatgaaaggaagggaagaaagcgaaagg

SEQ ID NO: 26 - DNA, 45 bp, synthetic construct
  misc_feature 1..45: note = primer LAEWXpr32-2-rv
  source 1..45: mol_type = other DNA
agtactcctaggactagtggtaccagatccggctgctaacaaagc

SEQ ID NO: 27 - DNA, 43 bp, synthetic construct
  misc_feature 1..43: note = primer LAEWXpr33-1-rv
  source 1..43: mol_type = other DNA
gttaatagtttgcgcaccaaatcggtgatgtcggcgatatagg

SEQ ID NO: 28 - DNA, 45 bp, synthetic construct
  misc_feature 1..45: note = primer LAEWXpr33-2-fr
  source 1..45: mol_type = other DNA
ggtaccactagtcctaggagtactatggctgctgcccatggtata

SEQ ID NO: 29 - DNA, 52 bp, synthetic construct
  misc_feature 1..52: note = primer LAEWXpr36-AIDA-fr
  source 1..52: mol_type = other DNA
aactttaagaaggagatataccatgggcaataaggcctacagtatcatttgg

SEQ ID NO: 30 - DNA, 45 bp, synthetic construct
  misc_feature 1..45: note = primer LAEWXpr37-AIDA-rv
  source 1..45: mol_type = other DNA
tttgttagcagccggatctgtcattatcagaagctgtattttatc

SEQ ID NO: 31 - DNA, 43 bp, synthetic construct
  misc_feature 1..43: note = primer LAEWXpr05 Forward
  source 1..43: mol_type = other DNA
agcgctgttccagggtccgggtaccactagagtctctatcttc

SEQ ID NO: 32 - DNA, 40 bp, synthetic construct
  misc_feature 1..40: note = primer LAEWXpr06 Reverse
  source 1..40: mol_type = other DNA
ccgccaccagaaccgcccggccagacatctctgaggtatc

SEQ ID NO: 33 - DNA, 20 bp, synthetic construct
  misc_feature 1..20: note = primer LAEWX15-FlexL-fr
  source 1..20: mol_type = other DNA
ccgggcggttctggtggcgg

SEQ ID NO: 34 - DNA, 20 bp, synthetic construct
  misc_feature 1..20: note = primer LAEWX16-FlexL-rvs
  source 1..20: mol_type = other DNA
cggagaaccgccgctaccgc

SEQ ID NO: 35 - DNA, 40 bp, synthetic construct
  misc_feature 1..40: note = primer LAEWXpr07 Forward
  source 1..40: mol_type = other DNA
gcggtagcggcggttctccgcctggcccaatcccggttcc

SEQ ID NO: 36 - DNA, 39 bp, synthetic construct
  misc_feature 1..39: note = primer LAEWXpr08 Reverse
  source 1..39: mol_type = other DNA
gaagtacaggttttcgagctcaaatgacctaggtttagc

SEQ ID NO: 37 - DNA, 24 bp, synthetic construct
  misc_feature 1..24: note = primer LAEWXpr35-amont-T7
  source 1..24: mol_type = other DNA
tccatccagtctattaattgttgc

SEQ ID NO: 38 - DNA, 24 bp, synthetic construct
  misc_feature 1..24: note = primer LAEWXpr22- ATR1-rv
  source 1..24: mol_type = other DNA
ccagacatctctgaggtatcttcc

SEQ ID NO: 39 - DNA, 24 bp, synthetic construct
  misc_feature 1..24: note = primer LAEWXpr24-2kbATR1-fr
  source 1..24: mol_type = other DNA
ggagcaggaaggtgtgagttcgtc

SEQ ID NO: 40 - DNA, 22 bp, synthetic construct
  misc_feature 1..22: note = primer LAEWXpr25-P540-rv
  source 1..22: mol_type = other DNA
accctggaagtacaggttttcg

SEQ ID NO: 41 - DNA, 35 bp, synthetic construct
  misc_feature 1..35: note = primer LAEWXpr03
  source 1..35: mol_type = other DNA
gctaaggaagctaaaatgaaatctaacaatgcgct

SEQ ID NO: 42 - DNA, 30 bp, synthetic construct
  misc_feature 1..30: note = primer LAEWXpr04
  source 1..30: mol_type = other DNA
tcgatggtgtcgccacgctgcccgagatgc

SEQ ID NO: 43 - DNA, 6580 bp, synthetic construct
  misc_feature 1..6580: note = plasmid pAIDA1-T7
  source 1..6580: mol_type = other DNA
aaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctgtcattatcagaagctgtattttatccccagtgctccggagatggcattgctcccgtgacctcctgcctgatatgcgactccgccattcactgacaagttttgagtaatcaccccttcaatacctgtctttatctctccctgatttcggctacctgacaacaactggctgtcatcactcattttaacaccaaattcatgagtgttatggatccagtttgcctctatatacggacggaacctccgcccggtatccttatccagggtgcttttcaccttccaggatgcacgaatacctgcttttgtctgaatattatttttccctgctccctgcaccaccgttccgttatcctcctgatgtgtatccggtgtaacccccatccagacagcctgcaaatgaggctgtaaccagaattcacctgttattccttcaggtgatgtccatgtgtgcacattcaggttatatcccccacctgcagaagcggttaaaccattcagattatatttttcttcttccagtccgtcacctttcactgatgcattaaaccagttatattgcatccaagtttcagcaaagagccctgttgcattttccccattctgataccacgtaccgtataccccgacagaataaccatccagtgtgtttctggcagctttgttgctcgtgtaatttatcgttttaccttttgcattcgcgtatcctcccataatccctaaggtaaaatcacccagttgttcagcatggaatttataaatatcccccccgagctgattgataaactgattggttgttgttttattttgcccgtcattcagcttaccagagcttattcctccagtgatcttcatccacacggatgcagactcaggctgtgtattatcactcatggccctgaattgcttacgctcattcaaatccatgaggaacagtgagttagccagtgccatattggtagcataacttccgttctccggtctgtattgccgggtatcagatgtgggaagatgactggttaaataccatcccttattatctgtcccactctcgtttcctttctgcagtgtgtaatcataagctccggcaactacgcggttcttcagagagaattctgcatcagaatttccctctacagaaataatattaataccatctctcgtctgaccaccactgccatcttcattgacataaacgatgtcactttgaccagaggtattacctttcaccaccagacggtccgtaagtgaattatctccttcaagcacaccaccaagagaaataacacttcccggtgtcccagtataatttgacacggtaagagtattacctatagcggccgcactttcttttgtaggattaagaatgatatttttgttattaacaagactaccattcactgtagcagagagcagctttgctgtaccaaaacttaagtcagcatcattcgtaatattcccgtttataatgctgttattaatgacaatgcttccattgttattcactctagacagatcttcttcgctaatcagtttctgttcaccctggaagtacaggttttcgagctccggaccctggaacagcgcttccagatggtgatggtgatggtggtcgactgcaaatgcatttccgattgtggaaacaaccgccaataccagcagtgtattttttgcaaggacaaaaccatgtcctctggctaactctgaggccacaatccaggcctgtctggagtggctccaaatgatactgtaggccttattgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatttggtgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagtatccgctcatgagattatcaaaaaggatcttcacctagatccttttgtaagttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgtggcgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaagtgagtggataaccgtattaccgcctttgagtgagctgataccgggaattctcactcattaggcatgatgatga

SEQ ID NO: 44 - DNA, 1982 bp, synthetic construct
  misc_feature 1..1982: note = cassette AIDA
  source 1..1982: mol_type = other DNA
taatacgactcactataggggaattgtgagcggataacaattcccctctagaaataattttgtttaactttaagaaggagatataccatgggcaataaggcctacagtatcatttggagccactccagacaggcctggattgtggcctcagagttagccagaggacatggttttgtccttgcaaaaaatacactgctggtattggcggttgtttccacaatcggaaatgcatttgcagtcgaccaccatcaccatcaccatctggaagcgctgttccagggtccgggtacccagaaacagcgtaccgagctcgaaaacctgtacttccagggtgaacagaaactgattagcgaagaagatctgtctagagtgaataacaatggaagcattgtcattaataacagcattataaacgggaatattacgaatgatgctgacttaagttttggtacagcaaagctgctctctgctacagtgaatggtagtcttgttaataacaaaaatatcattcttaatcctacaaaagaaagtgcggccgctataggtaatactcttaccgtgtcaaattatactgggacaccgggaagtgttatttctcttggtggtgtgcttgaaggagataattcacttacggaccgtctggtggtgaaaggtaatacctctggtcaaagtgacatcgtttatgtcaatgaagatggcagtggtggtcagacgagagatggtattaatattatttctgtagagggaaattctgatgcagaattctctctgaagaaccgcgtagttgccggagcttatgattacacactgcagaaaggaaacgagagtgggacagataataagggatggtatttaaccagtcatcttcccacatctgatacccggcaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagctgaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttctgataatgacagatccggctgctaacaaagcccgaaaggaagctgagttggctgctgccaccgctgagcaataactagcataaccccttggggcctctaaacgggtcttgaggggttttttg

SEQ ID NO: 45 - DNA, 969 bp, synthetic construct
  misc_feature 1..969: note = P450 CYP76F112
  source 1..969: mol_type = other DNA
caagtcacgacggttgtcatttcctccaccaccatggctaaagaagtcctccaggcaaacagccaagtcgtctccagccggacaatcaccgacgcaagccgcgcccacagacacagcgattttagcatggttatgttgcccgtatcccctctgtggcgaaaccttcggaaaataagcaactcacacttgctttcctccaaggctcttgatggcaacatggagctgagaaacaaaaaggtgcaagagctcctaaatgatgtccacaaaagcgtccaggccggggaggcggtggagatcgcgagcctttctttcagagctactctgaatctcttgtccaccacatttttctccatggacatggcggatgacacaaattccgtcactctaaaagagctcaaggaggctatgtcgcacatgatggaagagttggggaagcctaacttggccgattatttcccgtttctacaaaagattgacccccaaggcattaggcggcgcaacacggttactttccggaaactgatcaacttgtttgggcgtatcatcgaccaaagattgaaagtgagagaagcgagtggttctttgaaagatgatgatattttagacactcttatcaacatgatggtggtggatcaggagaagaaagaggatcagcttgacaaaaccataattgaacattttttactggatttattttcagcggggactgaaacgacttcaaccacgttggagtgggcaatggctgagctagtaaaagcgccagagattatgtcaaaagcccgagcagagctagatcaagttataggcaaaggaaaccaagtgaaggaatcggacgtatctcgactcccttacttacaagccattgttaaagaaaccttccgcatgcaccctacagctccattattgattcctcgcaaagccgacagtgacatcgaaatctccgactatatcatcccgaaggatgctcag

SEQ ID NO: 46 - protein, 503 aa, synthetic construct
  REGION 1..503: note = P450 CYP76F112
MDIFTSLLYLALILFFSLQVFRSFAFPKHKRLPPGPKPRPIIGSLLELGDQPHRSLARLSESYGPFMHLKLGQVTTVVISSTTMAKEVLQANSQVVSSRTITDASRAHRHSDFSMVMLPVSPLWRNLRKISNSHLLSSKALDGNMELRNKKVQELLNDVHKSVQAGEAVEIASLSFRATLNLLSTTFFSMDMADDTNSVTLKELKEAMSHMMEELGKPNLADYFPFLQKIDPQGIRRRNTVTFRKLINLFGRIIDQRLKVREASGSLKDDDILDTLINMMVVDQEKKEDQLDKTIIEHFLLDLFSAGTETTSTTLEWAMAELVKAPEIMSKARAELDQVIGKGNQVKESDVSRLPYLQAIVKETFRMHPTAPLLIPRKADSDIEISDYIIPKDAQVIVNVWAIGRDSSTWENPDKFIPERFLDIDIDVGGRDFKLIPFGAGRRICPGFPLAMRMLHLMLGSLLHSFDWKLEDGVRPDALNMDEKFGLTLQMAQPLRAIPVPTK

SEQ ID NO: 47 - protein, 505 aa, synthetic construct
  REGION 1..505: note = p450 CYP73A1
MDLLLIEKTLVALFAAIIGAILISKLRGKKFKLPPGPIPVPIFGNWLQVGDDLNHRNLTDLAKRFGEILLLRMGQRNLVVVSSPELAKEVLHTQGVEFGSRTRNVVFDIFTGKGQDMVFTVYGEHWRKMRRIMTVPFFTNKVVQQYRYGWEAEAAAVVDDVKKNPAAATEGIVIRRRLQLMMYNNMFRIMFDRRFESEDDPLFLKLKALNGERSRLAQSFEYNYGDFIPILRPFLRNYLKLCKEVKDKRIQLFKDYFVDERKKIGSTKKMDNNQLKCAIDHILEAKEKGEINEDNVLYIVENINVAAIETTLWSIEWGIAELVNHPEIQAKLRHELDTKLGPGVQITEPDVQNLPYLQAVVKETLRLRMAIPLLVPHMNLHDAKLGGFDIPAESKILVNAWWLANNPDQWKKPEEFRPERFLEEEAKVEANGNDFRYLPFGVGRRSCPGIILALPILGITIGRLVQNFELLPPPGQSKIDTDEKGGQFSLHILKHSTIVAKPRSF

SEQ ID NO: 48 - DNA, 33 bp, synthetic construct
  source 1..33: mol_type = other DNA; note = primer Vector 5 ATR1-FL-76F112 fwd
cgacaaagcatgagctcgaaaacctgtacttcc

SEQ ID NO: 49 - DNA, 30 bp, synthetic construct
  source 1..30: mol_type = other DNA; note = primer Vector New Vector 5 rvs
agactctagtggtacccggaccctggaaca

SEQ ID NO: 50 - DNA, 6607 bp, synthetic construct
  source 1..6607: mol_type = other DNA; note = plasmid pAIDA1-T7
agactctagtggtacccggaccctggaacagcgcttccagatggtgatggtgatggtggtcgactgcaaatgcatttccgattgtggaaacaaccgccaataccagcagtgtattttttgcaaggacaaaaccatgtcctctggctaactctgaggccacaatccaggcctgtctggagtggctccaaatgatactgtaggccttattgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatttggtgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagtatccgctcatgagattatcaaaaaggatcttcacctagatccttttgtaagttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgtggcgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaagtgagtggataaccgtattaccgcctttgagtgagctgataccgggaattctcactcattaggcatgatgatgaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctgtcattatcagaagctgtattttatccccagtgctccggagatggcattgctcccgtgacctcctgcctgatatgcgactccgccattcactgacaagttttgagtaatcaccccttcaatacctgtctttatctctccctgatttcggctacctgacaacaactggctgtcatcactcattttaacaccaaattcatgagtgttatggatccagtttgcctctatatacggacggaacctccgcccggtatccttatccagggtgcttttcaccttccaggatgcacgaatacctgcttttgtctgaatattatttttccctgctccctgcaccaccgttccgttatcctcctgatgtgtatccggtgtaacccccatccagacagcctgcaaatgaggctgtaaccagaattcacctgttattccttcaggtgatgtccatgtgtgcacattcaggttatatcccccacctgcagaagcggttaaaccattcagattatatttttcttcttccagtccgtcacctttcactgatgcattaaaccagttatattgcatccaagtttcagcaaagagccctgttgcattttccccattctgataccacgtaccgtataccccgacagaataaccatccagtgtgtttctggcagctttgttgctcgtgtaatttatcgttttaccttttgcattcgcgtatcctcccataatccctaaggtaaaatcacccagttgttcagcatggaatttataaatatcccccccgagctgattgataaactgattggttgttgttttattttgcccgtcattcagcttaccagagcttattcctccagtgatcttcatccacacggatgcagactcaggctgtgtattatcactcatggccctgaattgcttacgctcattcaaatccatgaggaacagtgagttagccagtgccatattggtagcataacttccgttctccggtctgtattgccgggtatcagatgtgggaagatgactggttaaataccatcccttattatctgtcccactctcgtttcctttctgcagtgtgtaatcataagctccggcaactacgcggttcttcagagagaattctgcatcagaatttccctctacagaaataatattaataccatctctcgtctgaccaccactgccatcttcattgacataaacgatgtcactttgaccagaggtattacctttcaccaccagacggtccgtaagtgaattatctccttcaagcacaccaccaagagaaataacacttcccggtgtcccagtataatttgacacggtaagagtattacctatagcggccgcactttcttttgtaggattaagaatgatatttttgttattaacaagactaccattcactgtagcagagagcagctttgctgtaccaaaacttaagtcagcatcattcgtaatattcccgtttataatgctgttattaatgacaatgcttccattgttattcactctagacagatcttcttcgctaatcagtttctgttcaccctggaagtacaggttttcgagctcatgctttgtcg

SEQ ID NO: 51 - DNA, 34 bp, synthetic construct
  source 1..34: mol_type = other DNA; note = primer ATR1 fwd 16123
tccgggtaccactagagtctctatcttcttcggt

SEQ ID NO: 52 - DNA, 33 bp, synthetic construct
  source 1..33: mol_type = other DNA; note = primer ATR1 rvs redo
ccgggagctcccagacatctctgaggtatcttc

SEQ ID NO: 53 - DNA, 31 bp, synthetic construct
  source 1..31: mol_type = other DNA; note = primer FLfwd redo
agatgtctgggagctcccgggcggttctggt

SEQ ID NO: 54 - DNA, 32 bp, synthetic construct
  source 1..32: mol_type = other DNA; note = primer FLrvs 76F112
ggacgaggtttctcgagcggagaaccgccgct

SEQ ID NO: 55 - DNA, 31 bp, synthetic construct
  source 1..31: mol_type = other DNA; note = primer 76F112 fwd
tccgctcgagaaacctcgtcccatcatcgga

SEQ ID NO: 56 - DNA, 30 bp, synthetic construct
  source 1..30: mol_type = other DNA; note = primer 76F112 rvs
tttcgagctcatgctttgtcggcacgggga

SEQ ID NO: 57 - DNA, 3419 bp, synthetic construct
  source 1..3419: mol_type = other DNA; note = merged sequence
tccgggtaccactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctgggagctcccgggcggttctggtggcggtagcggcggtggcggttctggcggtggcggtagcggcggtggcggttctggcggtggcggtagcggcggtggcggttctggcggtggcggtagcggcggtggcggttctggtggcggtagcggcggttctccgctcgagaaacctcgtcccatcatcggaagcctcttggagctcggcgaccaaccccacaggtccttggccaggctttccgagtcttacggcccgtttatgcatttgaagctcggccaagtcacgacggttgtcatttcctccaccaccatggctaaagaagtcctccaggcaaacagccaagtcgtctccagccggacaatcaccgacgcaagccgcgcccacagacacagcgattttagcatggttatgttgcccgtatcccctctgtggcgaaaccttcggaaaataagcaactcacacttgctttcctccaaggctcttgatggcaacatggagctgagaaacaaaaaggtgcaagagctcctaaatgatgtccacaaaagcgtccaggccggggaggcggtggagatcgcgagcctttctttcagagctactctgaatctcttgtccaccacatttttctccatggacatggcggatgacacaaattccgtcactctaaaagagctcaaggaggctatgtcgcacatgatggaagagttggggaagcctaacttggccgattatttcccgtttctacaaaagattgacccccaaggcattaggcggcgcaacacggttactttccggaaactgatcaacttgtttgggcgtatcatcgaccaaagattgaaagtgagagaagcgagtggttctttgaaagatgatgatattttagacactcttatcaacatgatggtggtggatcaggagaagaaagaggatcagcttgacaaaaccataattgaacattttttactggatttattttcagcggggactgaaacgacttcaaccacgttggagtgggcaatggctgagctagtaaaagcgccagagattatgtcaaaagcccgagcagagctagatcaagttataggcaaaggaaaccaagtgaaggaatcggacgtatctcgactcccttacttacaagccattgttaaagaaaccttccgcatgcaccctacagctccattattgattcctcgcaaagccgacagtgacatcgaaatctccgactatatcatcccgaaggatgctcaggtgattgtcaatgtatgggccattggtagagactcaagcacatgggaaaatcccgacaagtttataccggagaggtttttggacatcgatatagatgtcggaggccgggattttaagctcattccgttcggtgctggtcggagaatatgtcccggattcccattggcgatgcgaatgttgcacttgatgttggggtctttgcttcactcgtttgattggaagttggaagatggggttagacctgatgctctaaacatggatgaaaagtttggcctcaccttgcaaatggctcagcctttgcgagctatccccgtgccgacaaagcatgagctcgaaa

SEQ ID NO: 58 - DNA, 20 bp, synthetic construct
  source 1..20: mol_type = other DNA; note = primer 51F
cctgaataccgggtggtgac

SEQ ID NO: 59 - DNA, 22 bp, synthetic construct
  source 1..22: mol_type = other DNA; note = primer 52R
gcatatacacctacatggtcac

SEQ ID NO: 60 - DNA, 22 bp, synthetic construct
  source 1..22: mol_type = other DNA; note = primer 53F
gtgaaggagctcagaaggagta

SEQ ID NO: 61 - DNA, 21 bp, synthetic construct
  source 1..21: mol_type = other DNA; note = primer 54R
tagagttcggtggacgtccct

SEQ ID NO: 62 - DNA, 21 bp, synthetic construct
  source 1..21: mol_type = other DNA; note = primer 62F
tttgggcgtatcatcgaccaa

SEQ ID NO: 63 - DNA, 23 bp, synthetic construct
  source 1..23: mol_type = other DNA; note = primer 63R
atggttttgtcaagctgatcctc

SEQ ID NO: 64 - DNA, 21 bp, synthetic construct
  source 1..21: mol_type = other DNA; note = primer 57F
ttctcttggtggtgtgcttga

SEQ ID NO: 65 - DNA, 20 bp, synthetic construct
  source 1..20: mol_type = other DNA; note = primer 58R
catctctcgtctgaccacca

SEQ ID NO: 66 - DNA, 21 bp, synthetic construct
  source 1..21: mol_type = other DNA; note = primer 59F
ataacggaacggtggtgcagg

SEQ ID NO: 67 - DNA, 20 bp, synthetic construct
  source 1..20: mol_type = other DNA; note = primer 60R
aacctccgcccggtatcctt

SEQ ID NO: 68 - DNA, 22 bp, synthetic construct
  source 1..22: mol_type = other DNA; note = primer 61R
acgataccgaagacagctcatg

SEQ ID NO: 69 - DNA, 1404 bp, synthetic construct
  source 1..1404: mol_type = other DNA; note = sequence coding P450 CYP76F112
aaacctcgtcccatcatcggaagcctcttggagctcggcgaccaaccccacaggtccttggccaggctttccgagtcttacggcccgtttatgcatttgaagctcggccaagtcacgacggttgtcatttcctccaccaccatggctaaagaagtcctccaggcaaacagccaagtcgtctccagccggacaatcaccgacgcaagccgcgcccacagacacagcgattttagcatggttatgttgcccgtatcccctctgtggcgaaaccttcggaaaataagcaactcacacttgctttcctccaaggctcttgatggcaacatggagctgagaaacaaaaaggtgcaagagctcctaaatgatgtccacaaaagcgtccaggccggggaggcggtggagatcgcgagcctttctttcagagctactctgaatctcttgtccaccacatttttctccatggacatggcggatgacacaaattccgtcactctaaaagagctcaaggaggctatgtcgcacatgatggaagagttggggaagcctaacttggccgattatttcccgtttctacaaaagattgacccccaaggcattaggcggcgcaacacggttactttccggaaactgatcaacttgtttgggcgtatcatcgaccaaagattgaaagtgagagaagcgagtggttctttgaaagatgatgatattttagacactcttatcaacatgatggtggtggatcaggagaagaaagaggatcagcttgacaaaaccataattgaacattttttactggatttattttcagcggggactgaaacgacttcaaccacgttggagtgggcaatggctgagctagtaaaagcgccagagattatgtcaaaagcccgagcagagctagatcaagttataggcaaaggaaaccaagtgaaggaatcggacgtatctcgactcccttacttacaagccattgttaaagaaaccttccgcatgcaccctacagctccattattgattcctcgcaaagccgacagtgacatcgaaatctccgactatatcatcccgaaggatgctcaggtgattgtcaatgtatgggccattggtagagactcaagcacatgggaaaatcccgacaagtttataccggagaggtttttggacatcgatatagatgtcggaggccgggattttaagctcattccgttcggtgctggtcggagaatatgtcccggattcccattggcgatgcgaatgttgcacttgatgttggggtctttgcttcactcgtttgattggaagttggaagatggggttagacctgatgctctaaacatggatgaaaagtttggcctcaccttgcaaatggctcagcctttgcgagctatccccgtgccgacaaagcat

SEQ ID NO: 70 - protein, 468 aa, synthetic construct
  source 1..468: note = hydrophilic domain of a plant cytochrome P450
KPRPIIGSLLELGDQPHRSLARLSESYGPFMHLKLGQVTTVVISSTTMAKEVLQANSQVVSSRTITDASRAHRHSDFSMVMLPVSPLWRNLRKISNSHLLSSKALDGNMELRNKKVQELLNDVHKSVQAGEAVEIASLSFRATLNLLSTTFFSMDMADDTNSVTLKELKEAMSHMMEELGKPNLADYFPFLQKIDPQGIRRRNTVTFRKLINLFGRIIDQRLKVREASGSLKDDDILDTLINMMVVDQEKKEDQLDKTIIEHFLLDLFSAGTETTSTTLEWAMAELVKAPEIMSKARAELDQVIGKGNQVKESDVSRLPYLQAIVKETFRMHPTAPLLIPRKADSDIEISDYIIPKDAQVIVNVWAIGRDSSTWENPDKFIPERFLDIDIDVGGRDFKLIPFGAGRRICPGFPLAMRMLHLMLGSLLHSFDWKLEDGVRPDALNMDEKFGLTLQMAQPLRAIPVPTKH

SEQ ID NO: 71 - DNA, 1404 bp, synthetic construct; identical to SEQ ID NO: 69
  source 1..1404: mol_type = other DNA; note = polynucleotide encoding a polypeptide comprising a hydrophilic domain of a plant cytochrome P450

SEQ ID NO: 72 - DNA, 23 bp, synthetic construct
  source 1..23: mol_type = other DNA; note = primer 27F
gctagagtaagtagttcgccagt

(19) United States
(12) Patent Application Publication
(10) Pub. No.: US 2025/0320469 A1
(43) Pub. Date: Oct. 16, 2025

(54) FUSION PROTEIN AND USE FOR BIOCONVERTING MOLECULES

(71) Applicants: UNIVERSITE DE LORRAINE, Nancy (FR); CENTRE NATIONAL DE LA RECHERCHE SCIENTIFIQUE, PARIS (FR); INSTITUT NATIONAL DE RECHERCHE POUR L’AGRICULTURE L’ALIMENTATION ET L’ENVIRONNEMENT, Paris Cedex 07 (FR)

(72) Inventors: Xie WANG, Straßkirchen (DE); Alain HEHN, Neuves Maisons (FR); Mathieu ETIENNE, Vandoeuvre-lès-Nancy (FR)

(21) Appl. No.: 18/866,027

(22) PCT Filed: May 26, 2023
(86) PCT No.: PCT/FR2023/050742

§ 371 Date: Nov. 14, 2024

(30) Foreign Application Priority Data
May 30, 2022 (FR) FR2205144

(51) Int. Cl. C12N 9/02; C12N 15/62; C12N 15/70

(52) U.S. Cl. CPC C12N 9/0042; C12N 9/0079; C12N 15/62; C12N 15/70; C07K 2319/70; C12Y 106/02004; C12Y 114/15006

(57) ABSTRACT

The present invention relates to a fusion protein, to a nucleic acid coding for the protein, to a vector comprising the nucleic acid, to a host cell comprising the nucleic acid and/or vector, to a process for producing a fusion protein and to a process for bioconverting a substrate. The fusion protein of the present invention successively comprises (i) at least one polypeptide for targeting, and anchoring to, the bacterial membrane, (ii) at least one polypeptide corresponding to the hydrophilic domain of a plant P450 cytochrome, (iii) at least one binding polypeptide comprising at least 47 amino acids, and (iv) at least one polypeptide corresponding to the hydrophilic domain of a plant NADPH-dependent cytochrome P450 reductase.

### CROSS-REFERENCE TO RELATED APPLICATIONS

The present application is a filing under 35 U.S.C. 371 as the National Stage of International Application No. PCT/FR2023/050742, filed May 26, 2023, entitled “FUSION PROTEIN AND USE FOR BIOVCONVERTING MOLECULES,” which claims priority to French Application No. 2205144 filed with the Intellectual Property Office of France on May 30, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4692-19200 BNT231138USPC Sequence Listing.xml; created on Jun. 15, 2023; and having a file size of 130 KB.

The information in the Sequence Listing is incorporated herein in its entirety for all purposes.

### TECHNICAL FIELD OF THE INVENTION

The present invention relates to a fusion protein successively comprising (i) at least one polypeptide for targeting, and anchoring to, the bacterial membrane, (ii) at least one polypeptide corresponding to the hydrophilic domain of a plant P450 cytochrome, (iii) at least one binding polypeptide, and (iv) at least one polypeptide corresponding to the hydrophilic domain of a NADPH P450 reductase of cytochrome P450 of plant.

The present invention also relates to the nucleic acid coding for the fusion protein, the vector comprising said nucleic acid, the host cell comprising said nucleic acid and/or vector, and the process for producing said fusion protein.

The present invention also relates to a process for bioconverting a substrate comprising the use of a fusion protein.

The present invention finds application, in particular, in the field of protein and/or polypeptide production, molecule synthesis, e.g. bioconversion, and in the biological and/or medical field.

In the following description, references enclosed in brackets ([ ]) refer to the list of references presented at the end of the text.

### PRIOR ART

The specialized metabolism of plants is a metabolism of adaptation to changing environmental conditions. Over the course of its evolution, each plant has developed an arsenal of molecules enabling it to respond to its own living conditions. The diversity of molecules produced in this way is almost inexhaustible. These molecules, which can be highly complex, have been widely used by humans, notably in the healthcare industry.

The synthesis of these molecules is carried out via complex biosynthetic pathways involving numerous steps catalyzed by specific enzymes. P450 cytochromes (P450s) are among these enzymes, and can be considered high-precision tools for producing high-value-added molecules.

P450 cytochromes are therefore enzymes involved in numerous processes linked to the adaptation of plants to their environment. They originate some of the great diversity of molecules with remarkable physical/chemical properties not only in a physiological context, but also for human applications in various fields, including medicine, cosmetics, pharmaceuticals and agronomy. Molecular data on P450s has increased thanks to the use of high-throughput sequencing methods.

The data made available for many plants, particularly those with a reputation as medicinal plants, opens up prospects for the targeted production of molecules already identified or not yet identified/characterized.

The functional study of P450 cytochromes is, however, complex insofar as they are 1) membrane and intracellular proteins, 2) relatively fragile proteins, 3) proteins which, in order to be active, need to function in tandem with an NADPH P450 reductase supplying the electrons required for oxidation reactions.

To characterize these enzymes' functions, tools/processes have therefore been developed. As P450 cytochromes work in conjunction with NADPH P450 reductases, these two enzymes need to be in close interaction to enable electron transfer from the reductase to the P450 so that the reaction, i.e. an oxidation reaction, can take place. In plants, both enzymes are anchored in the endoplasmic reticulum membrane and are therefore intracellular. A known method for functional characterization of P450s comprises heterologous production of said P450 in yeast. Based on this production, a bioconversion approach was envisaged. To achieve this, a potential P450 substrate is added to the culture medium; said substrate penetrates the yeast and the resulting product can be stored in the yeast. Moreover, when the resulting product is stored in yeast, additional yeast lysis and purification steps are required to recover the product.

However, this process is not possible when the substrate is hydrophobic. Indeed, when it is hydrophobic, the substrate cannot enter the yeast (pass through the membrane) that is used.

There is therefore a real need to find a means and/or process enabling the production and/or functional characterization of P450 cytochromes and/or enabling the bioconversion of hydrophobic molecules or substrates by P450 cytochromes.

Another known method for functional characterization of P450s or conversion of molecules/substrates by P450s further comprises the removal of the cell wall and production of membrane extracts, that is, microsomes. Said microsomes are incubated in the presence of NADPH and potential substrates. This process comprises complex protein extraction steps, particularly for P450s. In addition, the numerous steps involved in cell wall removal and/or protein extraction mean that P450s are degraded, preventing multiple uses of P450s.

There is therefore a real need to find a means and/or process enabling the production and/or functional characterization of P450 cytochromes, while at the same time allowing the conservation of active P450 cytochromes. There is also a real need to find a bioconversion means and/or process that is reusable and/or does not comprise a protein extraction step.

### DESCRIPTION OF THE INVENTION

The aim of the present invention is precisely to meet these needs by providing a fusion protein successively comprising (i) at least one polypeptide for targeting, and anchoring to, the bacterial membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of a NADPH P450 reductase of cytochrome P450 of plant.

Surprisingly and unexpectedly, the inventors have demonstrated that the fusion protein according to the invention can be advantageously addressed to the bacterial plasma membrane and/or the outer membrane of bacteria, advantageously via its addressing sequence, for example in the form of a beta barrel. Furthermore, the inventors have surprisingly demonstrated that the fusion protein according to the invention targets the surface of said membranes, and advantageously the hydrophilic part is at the external surface of said membrane.

Advantageously, the inventors have demonstrated that once at the bacterial membrane, the portion of the fusion protein comprising a polypeptide comprising the hydrophilic domain of a plant P450 cytochrome, a binding polypeptide and a polypeptide comprising the hydrophilic domain of a NADPH P450 reductase of cytochrome P450 of plant is located outside the bacterial cell or bacterium and faces the external environment of the bacterium.

The inventors have also surprisingly demonstrated that the fusion protein according to the invention, advantageously when present on the outer surface of the cell membrane, can be used in substrate bioconversion processes.

The inventors have also surprisingly demonstrated that when the fusion protein according to the invention is used in a bioconversion process, it advantageously enables substrate bioconversion directly in the medium, advantageously outside the bacterium, advantageously enabling substrate bioconversion, advantageously avoiding steps of membrane protein extraction, advantageously limiting the risk of protein degradation during steps of protein purification, and enabling the production of molecules of interest directly in the culture medium, thus reducing steps of purification.

In the present, membrane means bacterial membrane. For example, this could be any membrane on the bacterial surface. For example, the outer membrane of gram-negative bacteria, or the plasma membrane of gram-positive bacteria. Advantageously, the bacterial membrane is the outer membrane of gram-negative bacteria.

In the present, a polypeptide for targeting, and anchoring to, the bacterial membrane means any polypeptide known to the person skilled in the art suitable for targeting and anchoring said polypeptide to the bacterial membrane. For example, it may be a polypeptide that targets and anchors to the plasma membrane of gram-positive bacteria. For example, it may be a polypeptide that targets and anchors to the external membrane of gram-negative bacteria. For example, it may be a polypeptide described in the document Jarmander, J., Gustavsson, M., Do, T H. et al. A dual tag system for facilitated detection of surface expressed proteins in Escherichia coli. Microb Cell Fact 11, 118 (2012). https://doi.org/10.1186/1475-2859-11-118 [14], For example, it may be a polypeptide whose quaternary structure forms a beta barrel. For example, it may be a polypeptide with a percent identity of at least 90%, for example of 91%, 92%, 93%, 94%, 95%, 96%, 97%, 98%, 99%, 100% with the polypeptide of sequence

(SEQ ID NO 1)
MNKAYSIIWSHSRQAWIVASELARGHGFVLAKNTLLVLAWSTIGNAF
AVDHHHHHHLEALFQGPGTQKQRTELENLYFQGEQKLISEEDLSRVN
NNGSIVINNSIINGNITNDADLSFGTAKLLSATVNGSLVNNKNIILN
PTKESAAAIGNTLTVSNYTGTPGSVISLGGVLEGDNSLTDRLWKGNT
SGQSDIVYVNEDGSGGQTRDGINIISVEGNSDAEFSLKNRWAGAYDY
TLQKGNESGTDNKGWYLTSHLPTSDTRQYRPENGSYATNMALANSLF
LMDLNERKQFRAMSDNTQPESASVWMKITGGISSGKLNDGQNKTTTN
QFINQLGGDIYKFHAEQLGDFTLGIMGGYANAKGKTINYTSNKAARN
TLDGYSVGVYGTWYQNGENATGLFAETWMQYNWFNASVKGDGLEEEK
YNLNGLTASAGGGYNLNVHTWTSPEGITGEFWLQPHLQAVWMGVTPD
THQEDNGTWQGAGKNNIQTKAGIRASWKVKSTLDKDTGRRFRPYIEA
NWIHNTHEFGVKMSDDSQLLSGSRNQGEIKTGIEGVITQNLSVNGGV
AYQAGGHGSNAISGALGIKYSF.

Advantageously, the polypeptide for targeting and anchoring to the membrane is a polypeptide of the sequence

(SEQ ID NO 1)
MNKAYSIIWSHSRQAWIVASELARGHGFVLAKNTLLVLAWSTIGNAF
AVDHHHHHHLEALFQGPGTQKQRTELENLYFQGEQKLISEEDLSRVN
NNGSIVINNSIINGNITNDADLSFGTAKLLSATVNGSLVNNKNIILN
PTKESAAAIGNTLTVSNYTGTPGSVISLGGVLEGDNSLTDRLWKGNT
SGQSDIVYVNEDGSGGQTRDGINIISVEGNSDAEFSLKNRWAGAYDY
TLQKGNESGTDNKGWYLTSHLPTSDTRQYRPENGSYATNMALANSLF
LMDLNERKQFRAMSDNTQPESASVWMKITGGISSGKLNDGQNKTTTN
QFINQLGGDIYKFHAEQLGDFTLGIMGGYANAKGKTINYTSNKAARN
TLDGYSVGVYGTWYQNGENATGLFAETWMQYNWFNASVKGDGLEEEK
YNLNGLTASAGGGYNLNVHTWTSPEGITGEFWLQPHLQAVWMGVTPD
THQEDNGTWQGAGKNNIQTKAGIRASWKVKSTLDKDTGRRFRPYIEA
NWIHNTHEFGVKMSDDSQLLSGSRNQGEIKTGIEGVITQNLSVNGGV
AYQAGGHGSNAISGALGIKYSF.

Advantageously, the inventors have demonstrated that the polypeptide for targeting, and anchoring to, the membrane effectively enables the fusion protein to be targeted and anchored to the external bacterial membrane, and advantageously also enables transport of the fusion protein from the cytosolic space to the external space of the bacterial cell. In addition, the peptide for targeting and anchoring to the membrane advantageously enables the fusion protein to pass through the membrane, thereby advantageously enabling the part of the fusion protein comprising a polypeptide comprising the hydrophilic domain of a plant P450 cytochrome, a binding polypeptide and a polypeptide comprising the hydrophilic domain of a NADPH P450 reductase of cytochrome P450 of plant to be located outside the bacterial cell or bacterium and face the external environment of the bacterium.

In the present, cytochrome P450 means proteins with mono-oxygenase activity capable of oxidizing substrates using molecular oxygen dissolved in the cytoplasm or in the medium, as well as the reducing equivalents provided by NADPH-cytochrome P450 reductase. (Guengerich and Macdonald, “Mechanisms of cytochrome P450 catalysis”, FASEB J. 1990, 4, pp 2453-2459 [1]). For example, it may be any plant P450 cytochrome known to the person skilled in the art. For example, they may be plant P450 cytochromes as described in Xu Jun et al. “The cytochrome P450 superfamily: Key players in plant development and defense” Journal of Integrative Agriculture 2015, 14(9): 1673-1686 [2], For example, they may be plant P450 cytochromes belonging to the CYP51, CYP71, CYP72, CYP74, CYP85, CYP86, CYP97, CYP710, CYP711 and CYP727 families. Examples include plant P450 cytochromes belonging to the CYP51, CYP71, CYP73, CYP75, CYP76, CYP77, CYP78, CYP79, CYP80, CYP81, CYP82, CYP83, CYP84, CYP89, CYP92, CYP93, CYP98, CYP99, CYP701, CYP703, CYP705, CYP706, CYP712, CYP719, CYP723, CYP726, CYP736, CYP72, CYP709, CYP714, CYP715, CYP721, CYP734, CYP735, CYP749, CYP74, CYP85, CYP87, CYP88, CYP90, CYP702, CYP707, CYP708, CYP716, CYP718, CYP720, CYP724, CYP725, CYP728, CYP729, CYP733, CYP86, CYP94, CYP96, CYP704, CYP730, CYP731, CYP732, CYP97, CYP710, CYP711 or CYP727 families. For example, it may be a P450 cytochrome belonging to the CYP76 or CYP73 family. For example, it may be a cytochrome P450 CYP76F112 or CYP73A1.

In the present, hydrophilic domain of a plant P450 cytochrome means the polypeptide sequence of the cytochrome P450 comprising the enzymatic domain and the biological activity, advantageously the enzymatic activity, of the cytochrome P450. The person skilled in the art, with this general knowledge, known how to identify the enzymatic domain of the P450 cytochrome. For example, it may be a polypeptide isolated from a P450 cytochrome. For example, it may be a polypeptide having a percent identity of at least 25%, for example 28%, 30%, 40%, 50%, 60%, 70%, 80%, 90%, 99% with a polypeptide selected from the group comprising

(SEQ ID NO 2)
HRNLTDLAKRFGEILLLRMGQRNLVVVSSPELAKEVLHTQGVEFGSR
TRNVVFDIFTGKGQDMVFTVYGEHWRKMRRIMTVPFFTNKVVQQYRY
GWEAEAAAVVDDVKKNPAAATEGIVIRRRLQLMMYNNMFRIMFDRRF
ESEDDPLFLKLKALNGERSRLAQSFEYNYGDFIPILRPFLRNYLKLC
KEVKDKRIQLFKDYFVDERKKIGSTKKMDNNQLKCAIDHILEAKEKG
EINEDNVLYIVENINVAAIETTLWSIEWGIAELVNHPEIQAKLRHEL
DTKLGPGVQITEPDVQNLPYLQAVVKETLRLRMAIPLLVPHMNLHDA
KLGGFDIPAESKILVNAWWLANNPDQWKKPEEFRPERFLEEEAKVEA
NGNDFRYLPFGVGRRSCPGIILALPILGITIGRLVQNFELLPPPGQS
KIDTDEKGGQFSLHILKH,
(SEQ ID NO 3)
IPVPIFGNWLQVGDDLNHRNLTDLAKRFGEILLLRMGQRNLVVVSSP
ELAKEVLHTQGVEFGSRTRNVVFDIFTGKGQDMVFTVYGEHWRKMRR
IMTVPFFTNKVVQQYRYGWEAEAAAVVDDVKKNPAAATEGIVIRRRL
QLMMYNNMFRIMFDRRFESEDDPLFLKLKALNGERSRLAQSFEYNYG
DFIPILRPFLRNYLKLCKEVKDKRIQLFKDYFVDERKKIGSTKKMDN
NQLKCAIDHILEAKEKGEINEDNVLYIVENINVAAIETTLWSIEWGI
AELVNHPEIQAKLRHELDTKLGPGVQITEPDVQNLPYLQAVVKETLR
LRMAIPLLVPHMNLHDAKLGGFDIPAESKILVNAWWLANNPDQWKKP
EEFRPERFLEEEAKVEANGNDFRYLPFGVGRRSCPGIILALPILGIT
IGRLVQNFELLPPPGQSKIDTDEKGGQFSLHILKHSTIVAKPRSF,
(SEQ ID NO 4)
MDIFTSLLYLALILFFSLQVFRSFAFPKHKRLPPGPKPRPIIGSLLE
LGDQPHRSLARLSESYGPFMHLKLGQVTTVVISSTTMAKEVLQANSQ
VVSSRTITDASRAHRHSDFSMVMLPVSPLWRNLRKISNSHLLSSKAL
DGNMELRNKKVQELLNDVHKSVQAGEAVEIASLSFRATLNLLSTTFF
SMDMADDTNSVTLKELKEAMSHMMEELGKPNLADYFPFLQKIDPQGI
RRRNTVTFRKLINLFGRIIDQRLKVREASGSLKDDDILDTLINMMVV
DQEKKEDQLDKTIIEHFLLDLFSAGTETTSTTLEWAMAELVKAPEIM
SKARAELDQVIGKGNQVKESDVSRLPYLQAIVKETFRMHPTAPLLIP
RKADSDIEISDYIIPKDAQ,
(SEQ ID NO 5)
KPRPIIGSLLELGDQPHRSLARLSESYGPFMHLKLGQVTTVVISSTT
MAKEVLQANSQVVSSRTITDASRAHRHSDFSMVMLPVSPLWRNLRKI
SNSHLLSSKALDGNMELRNKKVQELLNDVHKSVQAGEAVEIASLSFR
ATLNLLSTTFFSMDMADDTNSVTLKELKEAMSHMMEELGKPNLADYF
PFLQKIDPQGIRRRNTVTFRKLINLFGRIIDQRLKVREASGSLKDDD
ILDTLINMMVVDQEKKEDQLDKTIIEHFLLDLFSAGTETTSTTLEWA
MAELVKAPEIMSKARAELDQVIGKGNQVKESDVSRLPYLQAIVKETF
RMHPTAPLLIPRKADSDIEISDYIIPKDAQ,
(SEQ ID NO 70)
KPRPIIGSLLELGDQPHRSLARLSESYGPFMHLKLGQVTTVVISSTT
MAKEVLQANSQVVSSRTITDASRAHRHSDFSMVMLPVSPLWRNLRKI
SNSHLLSSKALDGNMELRNKKVQELLNDVHKSVQAGEAVEIASLSFR
ATLNLLSTTFFSMDMADDTNSVTLKELKEAMSHMMEELGKPNLADYF
PFLQKIDPQGIRRRNTVTFRKLINLFGRIIDQRLKVREASGSLKDDD
ILDTLINMMVVDQEKKEDQLDKTIIEHFLLDLFSAGTETTSTTLEWA
MAELVKAPEIMSKARAELDQVIGKGNQVKESDVSRLPYLQAIVKETF
RMHPTAPLLIPRKADSDIEISDYIIPKDAQVIVNVWAIGRDSSTWEN
PDKFIPERFLDIDIDVGGRDFKLIPFGAGRRICPGFPLAMRMLHLML
GSLLHSFDWKLEDGVRPDALNMDEKFGLTLQMAQPLRAIPVPTKH.

Advantageously, it can be a polypeptide isolated from a plant P450 cytochrome comprising the hydrophilic domain of said plant P450 cytochrome free of transmembrane domain. For example, it may be a polypeptide having a percent identity of at least 25%, for example 28%, 30%, 40%, 50%, 60%, 70%, 80%, 90%, 99% with a polypeptide selected from the group comprising

(SEQ ID NO 3)
IPVPIFGNWLQVGDDLNHRNLTDLAKRFGEILLLRMGQRNLVVVSSP
ELAKEVLHTQGVEFGSRTRNVVFDIFTGKGQDMVFTVYGEHWRKMRR
IMTVPFFTNKVVQQYRYGWEAEAAAVVDDVKKNPAAATEGIVIRRRL
QLMMYNNMFRIMFDRRFESEDDPLFLKLKALNGERSRLAQSFEYNYG
DFIPILRPFLRNYLKLCKEVKDKRIQLFKDYFVDERKKIGSTKKMDN
NQLKCAIDHILEAKEKGEINEDNVLYIVENINVAAIETTLWSIEWGI
AELVNHPEIQAKLRHELDTKLGPGVQITEPDVQNLPYLQAVVKETLR
LRMAIPLLVPHMNLHDAKLGGFDIPAESKILVNAWWLANNPDQWKKP
EEFRPERFLEEEAKVEANGNDFRYLPFGVGRRSCPGIILALPILGIT
IGRLVQNFELLPPPGQSKIDTDEKGGQFSLHILKHSTIVAKPRSF,
(SEQ ID NO 5)
KPRPIIGSLLELGDQPHRSLARLSESYGPFMHLKLGQVTTVVISSTT
MAKEVLQANSQVVSSRTITDASRAHRHSDFSMVMLPVSPLWRNLRKI
SNSHLLSSKALDGNMELRNKKVQELLNDVHKSVQAGEAVEIASLSFR
ATLNLLSTTFFSMDMADDTNSVTLKELKEAMSHMMEELGKPNLADYF
PFLQKIDPQGIRRRNTVTFRKLINLFGRIIDQRLKVREASGSLKDDD
ILDTLINMMVVDQEKKEDQLDKTIIEHFLLDLFSAGTETTSTTLEWA
MAELVKAPEIMSKARAELDQVIGKGNQVKESDVSRLPYLQAIVKETF
RMHPTAPLLIPRKADSDIEISDYIIPKDAQ,
et
(SEQ ID NO 70)
KPRPIIGSLLELGDQPHRSLARLSESYGPFMHLKLGQVTTVVISSTT
MAKEVLQANSQVVSSRTITDASRAHRHSDFSMVMLPVSPLWRNLRKI
SNSHLLSSKALDGNMELRNKKVQELLNDVHKSVQAGEAVEIASLSFR
ATLNLLSTTFFSMDMADDTNSVTLKELKEAMSHMMEELGKPNLADYF
PFLQKIDPQGIRRRNTVTFRKLINLFGRIIDQRLKVREASGSLKDDD
ILDTLINMMVVDQEKKEDQLDKTIIEHFLLDLFSAGTETTSTTLEWA
MAELVKAPEIMSKARAELDQVIGKGNQVKESDVSRLPYLQAIVKETF
RMHPTAPLLIPRKADSDIEISDYIIPKDAQVIVNVWAIGRDSSTWEN
PDKFIPERFLDIDIDVGGRDFKLIPFGAGRRICPGFPLAMRMLHLML
GSLLHSFDWKLEDGVRPDALNMDEKFGLTLQMAQPLRAIPVPTKH.

It may be a polypeptide isolated from a plant P450 cytochrome comprising the hydrophilic domain of said plant P450 cytochrome selected from the group comprising the polypeptide

(SEQ ID NO 3)
IPVPIFGNWLQVGDDLNHRNLTDLAKRFGEILLLRMGQRNLVVVSSP
ELAKEVLHTQGVEFGSRTRNVVFDIFTGKGQDMVFTVYGEHWRKMRR
IMTVPFFTNKVVQQYRYGWEAEAAAVVDDVKKNPAAATEGIVIRRRL
QLMMYNNMFRIMFDRRFESEDDPLFLKLKALNGERSRLAQSFEYNYG
DFIPILRPFLRNYLKLCKEVKDKRIQLFKDYFVDERKKIGSTKKMDN
NQLKCAIDHILEAKEKGEINEDNVLYIVENINVAAIETTLWSIEWGI
AELVNHPEIQAKLRHELDTKLGPGVQITEPDVQNLPYLQAVVKETLR
LRMAIPLLVPHMNLHDAKLGGFDIPAESKILVNAWWLANNPDQWKKP
EEFRPERFLEEEAKVEANGNDFRYLPFGVGRRSCPGIILALPILGIT
IGRLVQNFELLPPPGQSKIDTDEKGGQFSLHILKHSTIVAKPRSF
et
(SEQ ID NO 70)
KPRPIIGSLLELGDQPHRSLARLSESYGPFMHLKLGQVTTVVISSTT
MAKEVLQANSQVVSSRTITDASRAHRHSDFSMVMLPVSPLWRNLRKI
SNSHLLSSKALDGNMELRNKKVQELLNDVHKSVQAGEAVEIASLSFR
ATLNLLSTTFFSMDMADDTNSVTLKELKEAMSHMMEELGKPNLADYF
PFLQKIDPQGIRRRNTVTFRKLINLFGRIIDQRLKVREASGSLKDDD
ILDTLINMMVVDQEKKEDQLDKTIIEHFLLDLFSAGTETTSTTLEWA
MAELVKAPEIMSKARAELDQVIGKGNQVKESDVSRLPYLQAIVKETF
RMHPTAPLLIPRKADSDIEISDYIIPKDAQVIVNVWAIGRDSSTWEN
PDKFIPERFLDIDIDVGGRDFKLIPFGAGRRICPGFPLAMRMLHLML
GSLLHSFDWKLEDGVRPDALNMDEKFGLTLQMAQPLRAIPVPTKH.

In the present by binding polypeptide means as any suitable binding polypeptide known to the person skilled in the art. For example, it may be a binding polypeptide comprising at least 47 amino acids, preferably comprising 51 amino acids. For example, it could be a polypeptide with the sequence

(SEQ ID NO 6)
PGGSGGGSGGGGSGGGGSGGGGSGGGGSGGGGSGGGGSGGGGSGGGS
GGSP.

In the present, NADPH P450 reductase of cytochrome P450 of plant means proteins with oxidoreductase activity that catalyze the reaction:

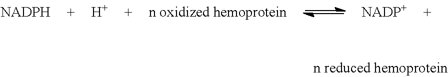

For example, it may be any NADPH P450 reductase of cytochrome P450 of plant known to the person skilled in the art. One example it may be the NADPH P450 reductase of cytochrome P450 of plant described in Kenneth Jensen et al, “Plant NADPH-cytochrome P450 oxidoreductases”, Phytochemistry 2010, Volume 71, 2-3, Pages 132-141 [3],

In the present, hydrophilic domain of NADPH P450 reductase of cytochrome P450 of plant means the polypeptide sequence of NADPH P450 reductase of cytochrome P450 of plant comprising the reductase domain and the biological activity, advantageously the reducing activity, of plant NADPH P450 reductase of cytochrome P450 of plant. The term “reductase domain”, as used here, refers to an amino acid sequence that functions as an electron donor. In particular, it acts as an electron donor for the oxygenase part of a cytochrome P450. The person skilled in the art, by this general knowledge, knows how to identify the catalytic domain, in particular the reductase domain, of a NADPH P450 reductase of cytochrome P450 of plant. For example, it may be a polypeptide isolated from a NADPH P450 reductase of cytochrome P450 of plant. For example, it may be a polypeptide with a percent identity of at least 90%, for example 91%, 92%, 93%, 94%, 95%, 96%, 97%, 98%, 99%, 100% with a polypeptide selected from the group comprising

(SEQ ID NO 7)
TRVSIFFGTQTGTAEGFAKALSEEIKARYEKAAVKVIDLDDYAADDD
QYEEKLKKETLAFFCVATYGDGEPTDNAARFYKWFTEENERDIKLQQ
LAYGVFALGNRQYEHFNKIGIVLDEELCKKGAKRLIEVGLGDDDQSI
EDDFNAWKESLWSELDKLLKDEDDKSVATPYTAVIPEYRVVTHDPRF
TTQKSMESNVANGNTTIDIHHPCRVDVAVQKELHTHESDRSCIHLEF
DISRTGITYETGDHVGVYAENHVEIVEEAGKLLGHSLDLVFSIHADK
EDGSPLESAVPPPFPGPCTLGTGLARYADLLNPPRKSALVALAAYAT
EPSEAEKLKHLTSPDGKDEYSQWIVASQRSLLEVMAAFPSAKPPLGV
FFAAIAPRLQPRYYSISSSPRLAPSRVHVTSALVYGPTPTGRIHKGV
CSTWMKNAVPAEKSHECSGAPIFIRASNFKLPSNPSTPIVMVGPGTG
LAPFRGFLQERMALKEDGEELGSSLLFFGCRNRQMDFIYEDELNNFV
DQGVISELIMAFSREGAQKEYVQHKMMEKAAQVWDLIKEEGYLYVCG
DAKGMARDVHRTLHTIVQEQEGVSSSEAEAIVKKLQTEGRYLRDVW,
(SEQ ID NO 8)
YEKAAVKVIDLDDYAADDDQYEEKLKKETLAFFCVATYGDGEPTDNA
ARFYKWFTEENERDIKLQQLAYGVFALGNRQYEHFNKIGIVLDEELC
KKGAKRLIEVGLGDDDQSIEDDFNAWKESLWSELDKLLKDEDDKSVA
TPYTAVIPEYRVVTHDPRFTTQKSMESNVANGNTTIDIHHPCRVDVA
VQKELHTHESDRSCIHLEFDISRTGITYETGDHVGVYAENHVEIVEE
AGKLLGHSLDLVFSIHADKEDGSPLESAVPPPFPGPCTLGTGLARYA
DLLNPPRKSALVALAAYATEPSEAEKLKHLTSPDGKDEYSQWIVASQ
RSLLEVMAAFPSAKPPLGVFFAAIAPRLQPRYYSISSSPRLAPSRVH
VTSALVYGPTPTGRIHKGVCSTWMKNAVPAEKSHECSGAPIFIRASN
FKLPSNPSTPIVMVGPGTGLAPFRGFLQERMALKEDGEELGSSLLFF
GCRNRQMDFIYEDELNNFVDQGVISELIMAFSREGAQKEYVQHKMME
KAAQVWDLIKEEGYLYVCGDAKGMARDVHRTLHTIVQEQEGVSSSE.

Advantageously, it may be a polypeptide isolated from a NADPH P450 reductase of cytochrome P450 comprising the hydrophilic domain of said NADPH P450 reductase of cytochrome P450 of plant free of transmembrane domain. For example, it may be a polypeptide with a percent identity of at least 90%, for example 91%, 92%, 93%, 94%, 95%, 96%, 97%, 98%, 99%, 100% with the polypeptide of sequence

(SEQ ID NO 7)
TRVSIFFGTQTGTAEGFAKALSEEIKARYEKAAVKVIDLDDYAADDD
QYEEKLKKETLAFFCVATYGDGEPTDNAARFYKWFTEENERDIKLQQ
LAYGVFALGNRQYEHFNKIGIVLDEELCKKGAKRLIEVGLGDDDQSI
EDDFNAWKESLWSELDKLLKDEDDKSVATPYTAVIPEYRVVTHDPRF
TTQKSMESNVANGNTTIDIHHPCRVDVAVQKELHTHESDRSCIHLEF
DISRTGITYETGDHVGVYAENHVEIVEEAGKLLGHSLDLVFSIHADK
EDGSPLESAVPPPFPGPCTLGTGLARYADLLNPPRKSALVALAAYAT
EPSEAEKLKHLTSPDGKDEYSQWIVASQRSLLEVMAAFPSAKPPLGV
FFAAIAPRLQPRYYSISSSPRLAPSRVHVTSALVYGPTPTGRIHKGV
CSTWMKNAVPAEKSHECSGAPIFIRASNFKLPSNPSTPIVMVGPGTG
LAPFRGFLQERMALKEDGEELGSSLLFFGCRNRQMDFIYEDELNNFV
DQGVISELIMAFSREGAQKEYVQHKMMEKAAQVWDLIKEEGYLYVCG
DAKGMARDVHRTLHTIVQEQEGVSSSEAEAIVKKLQTEGRYLRDVW.

Advantageously, the inventors have demonstrated that the binding polypeptide makes it possible to obtain a fusion protein with a quaternary structure enabling functional enzymatic activity of the hydrophilic domain of a plant P450 cytochrome and the hydrophilic domain of NADPH P450 reductase of cytochrome P450 of plant, advantageously enabling effective bioconversion of substrates of different structures and sizes.

In the present, the fusion protein may comprise one or more unnatural amino acids, for example, one or more D-amino acids and/or chemically modified amino acids.

Unnatural amino acids can be levorotatory (L-), dextrorotatory (D-), or mixtures thereof. Unnatural amino acids are those which are generally not synthesized in the normal metabolic processes of living organisms, and which are not naturally present in proteins. In addition, the unnatural amino acids are not recognized by common proteases. The unnatural amino acid can be present at any position in the fusion protein. For example, the unnatural amino acid may be located at the N-terminus, the C-terminus or any position between the N-terminus and the C-terminus.

The unnatural amino acids may, for example, be chemically modified amino acids and may, for example, include alkyl, aryl, or alkylaryl groups not found in natural amino acids. Some examples of unnatural alkylated amino acids comprise α-aminobutyric acid, β-aminobutyric acid, γ-aminobutyric acid, 8-aminovaleric acid, and ε-aminocaproic acid. Some examples of unnatural aryl amino acids comprise ortho-, meta- and para-aminobenzoic acid. Some examples of non-natural alkylaryl amino acids comprise ortho-, meta- and para-aminophenylacetic acid, and γ-phenyl-β-aminobutyric acid. Unnatural amino acids comprise derivatives of natural amino acids. Natural amino acid derivatives may, for example, comprise the addition of one or more chemical groups to the natural amino acid. For example, one or more chemical groups can be added to one or more of the 2′, 3′, 4′, 5′ or 6′ positions of the aromatic ring of a phenylalanine or tyrosine residue, or to the 4′, 5′, 6′ or 7′ position of the benzo ring of a tryptophan residue. The group can be any chemical group that can be added to an aromatic ring. Some examples of such groups include branched or unbranched C1-C4 alkyl, such as methyl, ethyl, n-propyl, isopropyl, butyl, isobutyl or t-butyl, C1-C4 alkyloxy (that is, alkoxy), amino, C1-C4 alkylamino and C1-C4 dialkylamino (e.g. methylamino, dimethylamino), nitro, hydroxyl, halo (that is, fluoro, chloro, bromo or iodo). Specific examples of non-natural derivatives of natural amino acids include norvaline (Nva) and norleucine (Nie).

The fusion protein according to the invention can be produced and/or synthesized by any suitable process known to the person skilled in the art.

In the present, the terms “polypeptide”, “peptide” and their grammatical equivalents refer to a polymer of amino acid residues.

In the present, a “functional protein” is a protein that is biologically active.

In the present, the percentage sequence identity can be determined by any method known to the person skilled in the art. It can be determined by using BLASTP and BLASTN, for example, or by using default settings.

In the present, the expression “percent identity” means the percentage determined by direct comparison of two sequences (nucleic or protein), by determining the number of nucleotides or amino acid residues common to the two sequences, then dividing by the number of nucleotides or amino acid residues of the longer of the two sequences and multiplying the result by 100.

In the present, the word “comprises” and its variations, such as “comprises” and “comprising”, are to be interpreted in an open and inclusive sense, that is, as “including, but not limited to”.

In the present, the term “consisting of” or “constituted” means including, and limited to, whatever follows the expression “consisting of” or “constituted”. Thus, the expression “consisting of” or “constituted” indicates that the elements listed are required or mandatory, and that no other elements may be present.

Another object of the invention relates to a polynucleotide or nucleic acid coding for a fusion protein according to the invention.

For example, this may be a polynucleotide or nucleic acid coding for a fusion protein successively comprising (i) at least one polypeptide for targeting, and anchoring to, the bacterial membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of a NADPH P450 reductase of cytochrome P450 of plant.

It may be, for example, a polynucleotide or nucleic acid coding for a fusion protein according to the invention wherein the polynucleotide sequence or nucleic acid coding for a polypeptide for addressing and anchoring to the bacterial membrane, advantageously to the outer membrane of gram-negative bacteria, is selected from the group comprising the nucleic acid of sequence:

(SEQ ID NO 9)
accatgggcaataaggcctacagtatcatttggagccactccagaca
ggcctggattgtggcctcagagttagccagaggacatggttttgtcc
ttgcaaaaaatacactgctggtattggcggttgtttccacaatcgga
aatgcatttgcagtcgaccaccatcaccatcaccatctggaagcgct
gttccagggtccgggtaccgctacagtgaatggtagtcttgttaata
acaaaaatatcattcttaatcctacaaaagaaagtgcggccgctata
ggtaatactcttaccgtgtcaaattatactgggacaccgggaagtgt
tatttctcttggtggtgtgcttgaaggagataattcacttacggacc
gtctggtggtgaaaggtaatacctctggtcaaagtgacatcgtttat
gtcaatgaagatggcagtggtggtcagacgagagatggtattaatat
tatttctgtagagggaaattctgatgcagaattctctctgaagaacc
gcgtagttgccggagcttatgattacacactgcagaaaggaaacgag
agtgggacagataataagggatggtatttaaccagtcatcttcccac
atctgatacccggcaatacagaccggagaacggaagttatgctacca
atatggcactggctaactcactgttcctcatggatttgaatgagcgt
aagcaattcagggccatgagtgataatacacagcctgagtctgcatc
cgtgtggatgaagatcactggaggaataagctctggtaagctgaatg
acgggcaaaataaaacaacaaccaatcagtttatcaatcagctcggg
ggggatatttataaattccatgctgaacaactgggtgattttacctt
agggattatgggaggatacgcgaatgcaaaaggtaaaacgataaatt
acacgagcaacaaagctgccagaaacacactggatggttattctgtc
ggggtatacggtacgtggtatcagaatggggaaaatgcaacagggct
ctttgctgaaacttggatgcaatataactggtttaatgcatcagtga
aaggtgacggactggaagaagaaaaatataatctgaatggtttaacc
gcttctgcaggtgggggatataacctgaatgtgcacacatggacatc
acctgaaggaataacaggtgaattctggttacagcctcatttgcagg
ctgtctggatgggggttacaccggatacacatcaggaggataacgga
acggtggtgcagggagcagggaaaaataatattcagacaaaagcagg
tattcgtgcatcctggaaggtgaaaagcaccctggataaggataccg
ggcggaggttccgtccgtatatagaggcaaactggatccataacact
catgaatttggtgttaaaatgagtgatgacagccagttgttgtcagg
tagccgaaatcagggagagataaagacaggtattgaaggggtgatta
ctcaaaacttgtcagtgaatggcggagtcgcatatcaggcaggaggt
cacgggagcaatgccatctccggagcactggggataaaatacagctt
ctgataatga,
(SEQ ID NO 10)
atgaataaggcctacagtatcatttggagccactccagacaggcctg
gattgtggcctcagagttagccagaggacatggttttgtccttgcaa
aaaatacactgctggtattggcggttgtttccacaatcggaaatgca
tttgcagtcgaccaccatcaccatcaccatctggaagcgctgttcca
gggtccgggtacccagaaacagcgtaccgagctcgaaaacctgtact
tccagggtgaacagaaactgattagcgaagaagatctgtctagagtg
aataacaatggaagcattgtcattaataacagcattataaacgggaa
tattacgaatgatgctgacttaagttttggtacagcaaagctgctct
ctgctacagtgaatggtagtcttgttaataacaaaaatatcattctt
aatcctacaaaagaaagtgcggccgctataggtaatactcttaccgt
gtcaaattatactgggacaccgggaagtgttatttctcttggtggtg
tgcttgaaggagataattcacttacggaccgtctggtggtgaaaggt
aatacctctggtcaaagtgacatcgtttatgtcaatgaagatggcag
tggtggtcagacgagagatggtattaatattatttctgtagagggaa
attctgatgcagaattctctctgaagaaccgcgtagttgccggagct
tatgattacacactgcagaaaggaaacgagagtgggacagataataa
gggatggtatttaaccagtcatcttcccacatctgatacccggcaat
acagaccggagaacggaagttatgctaccaatatggcactggctaac
tcactgttcctcatggatttgaatgagcgtaagcaattcagggccat
gagtgataatacacagcctgagtctgcatccgtgtggatgaagatca
ctggaggaataagctctggtaagctgaatgacgggcaaaataaaaca
acaaccaatcagtttatcaatcagctcgggggggatatttataaatt
ccatgctgaacaactgggtgattttaccttagggattatgggaggat
acgcgaatgcaaaaggtaaaacgataaattacacgagcaacaaagct
gccagaaacacactggatggttattctgtcggggtatacggtacgtg
gtatcagaatggggaaaatgcaacagggctctttgctgaaacttgga
tgcaatataactggtttaatgcatcagtgaaaggtgacggactggaa
gaagaaaaatataatctgaatggtttaaccgcttctgcaggtggggg
atataacctgaatgtgcacacatggacatcacctgaaggaataacag
gtgaattctggttacagcctcatttgcaggctgtctggatgggggtt
acaccggatacacatcaggaggataacggaacggtggtgcagggagc
agggaaaaataatattcagacaaaagcaggtattcgtgcatcctgga
aggtgaaaagcaccctggataaggataccgggcggaggttccgtccg
tatatagaggcaaactggatccataacactcatgaatttggtgttaa
aatgagtgatgacagccagttgttgtcaggtagccgaaatcagggag
agataaagacaggtattgaaggggtgattactcaaaacttgtcagtg
aatggcggagtcgcatatcaggcaggaggtcacgggagcaatgccat
ctccggagcactggggataaaatacagcttctgataatga.

For example, it may be a polynucleotide or nucleic acid coding for a fusion protein according to the invention wherein the polynucleotide sequence or nucleic acid coding for a polypeptide comprising the hydrophilic domain of a plant P450 cytochrome is selected from the group comprising the nucleic acid of sequence:

(SEQ ID NO 11)
cctggcccaatcccggttccaattttcggcaactggctacaagttgg
cgatgatttgaaccaccggaacttaaccgatctggctaagaggtttg
gtgagatcttgctgctacgcatggggcagaggaatctggtagttgtg
tcttcgcctgagcttgctaaagaggtgttgcatacacaaggagtgga
gtttggttcgagaacaaggaatgttgtgttcgatatttttactggga
agggtcaggatatggtgtttacggtttatggtgagcattggaggaag
atgaggaggatcatgaccgtaccctttttcaccaacaaagttgttca
gcaatacaggtatgggtgggaggctgaggccgcggcggttgtggacg
atgtgaagaagaatccggctgcagcaactgaaggaatcgtgatccga
agacggttacaactcatgatgtataacaacatgttcagaatcatgtt
cgacagacgattcgaaagtgaagatgatcccttgtttttgaaactca
aggcgttgaacggtgagaggagtcgattggcgcagagctttgagtac
aactatggcgatttcatccctattttgcggccgtttttgagaaatta
tttgaagttgtgcaaggaagttaaagataaaaggattcagctcttca
aggattacttcgttgacgaaaggaagaagattggaagcactaagaaa
atggacaacaatcagttgaaatgtgccattgatcacattcttgaagc
taaagagaagggtgagatcaatgaagacaatgttctttacattgttg
aaaacatcaatgttgcagcaatcgagacaactctatggtcgatcgaa
tggggaattgcggagctagttaaccatcccgagatccaagccaaact
caggcacgagctcgacaccaagctcgggcccggtgtccagatcaccg
agcccgacgtccaaaacctcccttacctccaagccgtggtcaaggaa
accctccgtctccgtatggcgatcccgcttctagtcccacacatgaa
cctccatgacgctaagctcggcgggtttgacatcccggccgaaagca
agatcttggtcaacgcgtggtggttagcaaacaaccccgaccaatgg
aagaaacccgaggagtttaggccagagaggtttttggaagaggaagc
gaaggttgaggctaacgggaatgattttaggtacttgccgtttggag
tcgggagaaggagttgccccgggattattcttgcattgccgatactt
ggtattacaatcgggcgtttggtgcagaatttcgagctgttgcctcc
accgggacagtctaagatcgataccgatgagaagggtgggcagttta
gtttgcatatcttgaagcactctactatcgtagctaaacctaggtca
ttt,
(SEQ ID NO 12)
atggacctcctcctcatagaaaaaaccctcgtcgccttattcgccgc
cattatcggcgcaatactaatctccaaactccgcggtaaaaaattca
agctcccacctggcccaatcccggttccaattttcggcaactggcta
caagttggcgatgatttgaaccaccggaacttaaccgatctggctaa
gaggtttggtgagatcttgctgctacgcatggggcagaggaatctgg
tagttgtgtcttcgcctgagcttgctaaagaggtgttgcatacacaa
ggagtggagtttggttcgagaacaaggaatgttgtgttcgatatttt
tactgggaagggtcaggatatggtgtttacggtttatggtgagcatt
ggaggaagatgaggaggatcatgaccgtaccctttttcaccaacaaa
gttgttcagcaatacaggtatgggtgggaggctgaggccgcggcggt
tgtggacgatgtgaagaagaatccggctgcagcaactgaaggaatcg
tgatccgaagacggttacaactcatgatgtataacaacatgttcaga
atcatgttcgacagacgattcgaaagtgaagatgatcccttgttttt
gaaactcaaggcgttgaacggtgagaggagtcgattggcgcagagct
ttgagtacaactatggcgatttcatccctattttgcggccgtttttg
agaaattatttgaagttgtgcaaggaagttaaagataaaaggattca
gctcttcaaggattacttcgttgacgaaaggaagaagattggaagca
ctaagaaaatggacaacaatcagttgaaatgtgccattgatcacatt
cttgaagctaaagagaagggtgagatcaatgaagacaatgttcttta
cattgttgaaaacatcaatgttgcagcaatcgagacaactctatggt
cgatcgaatggggaattgcggagctagttaaccatcccgagatccaa
gccaaactcaggcacgagctcgacaccaagctcgggcccggtgtcca
gatcaccgagcccgacgtccaaaacctcccttacctccaagccgtgg
tcaaggaaaccctccgtctccgtatggcgatcccgcttctagtccca
cacatgaacctccatgacgctaagctcggcgggtttgacatcccggc
cgaaagcaagatcttggtcaacgcgtggtggttagcaaacaaccccg
accaatggaagaaacccgaggagtttaggccagagaggtttttggaa
gaggaagcgaaggttgaggctaacgggaatgattttaggtacttgcc
gtttggagtcgggagaaggagttgccccgggattattcttgcattgc
cgatacttggtattacaatcgggcgtttggtgcagaatttcgagctg
ttgcctccaccgggacagtctaagatcgataccgatgagaagggtgg
gcagtttagtttgcatatcttgaagcactctactatcgtagctaaac
ctaggtcattttaa,
(SEQ ID NO 13)
atggatattttcacctccttactgtatcttgctctcattcttttctt
ttctcttcaagtcttccgttcctttgcgtttcctaaacacaaaaggc
ttccacctggtccaaaacctcgtcccatcatcggaagcctcttggag
ctcggcgaccaaccccacaggtccttggccaggctttccgagtctta
cggcccgtttatgcatttgaagctcggccaagtcacgacggttgtca
tttcctccaccaccatggctaaagaagtcctccaggcaaacagccaa
gtcgtctccagccggacaatcaccgacgcaagccgcgcccacagaca
cagcgattttagcatggttatgttgcccgtatcccctctgtggcgaa
accttcggaaaataagcaactcacacttgctttcctccaaggctctt
gatggcaacatggagctgagaaacaaaaaggtgcaagagctcctaaa
tgatgtccacaaaagcgtccaggccggggaggcggtggagatcgcga
gcctttctttcagagctactctgaatctcttgtccaccacatttttc
tccatggacatggcggatgacacaaattccgtcactctaaaagagct
caaggaggctatgtcgcacatgatggaagagttggggaagcctaact
tggccgattatttcccgtttctacaaaagattgacccccaaggcatt
aggcggcgcaacacggttactttccggaaactgatcaacttgtttgg
gcgtatcatcgaccaaagattgaaagtgagagaagcgagtggttctt
tgaaagatgatgatattttagacactcttatcaacatgatggtggtg
gatcaggagaagaaagaggatcagcttgacaaaaccataattgaaca
ttttttactggatttattttcagcggggactgaaacgacttcaacca
cgttggagtgggcaatggctgagctagtaaaagcgccagagattatg
tcaaaagcccgagcagagctagatcaagttataggcaaaggaaacca
agtgaaggaatcggacgtatctcgactcccttacttacaagccattg
ttaaagaaaccttccgcatgcaccctacagctccattattgattcct
cgcaaagccgacagtgacatcgaaatctccgactatatcatcccgaa
ggatgctcaggtgattgtcaatgtatgggccattggtagagactcaa
gcacatgggaaaatcccgacaagtttataccggagaggtttttggac
atcgatatagatgtcggaggccgggattttaagctcattccgttcgg
tgctggtcggagaatatgtcccggattcccattggcgatgcgaatgt
tgcacttgatgttggggtctttgcttcactcgtttgattggaagttg
gaagatggggttagacctgatgctctaaacatggatgaaaagtttgg
cctcaccttgcaaatggctcagcctttgcgagctatccccgtgccga
caaagcattag,
(SEQ ID NO 14)
atcatcggaagcctcttggagctcggcgaccaaccccacaggtcctt
ggccaggctttccgagtcttacggcccgtttatgcatttgaagctcg
gccaagtcacgacggttgtcatttcctccaccaccatggctaaagaa
gtcctccaggcaaacagccaagtcgtctccagccggacaatcaccga
cgcaagccgcgcccacagacacagcgattttagcatggttatgttgc
ccgtatcccctctgtggcgaaaccttcggaaaataagcaactcacac
ttgctttcctccaaggctcttgatggcaacatggagctgagaaacaa
aaaggtgcaagagctcctaaatgatgtccacaaaagcgtccaggccg
gggaggcggtggagatcgcgagcctttctttcagagctactctgaat
ctcttgtccaccacatttttctccatggacatggcggatgacacaaa
ttccgtcactctaaaagagctcaaggaggctatgtcgcacatgatgg
aagagttggggaagcctaacttggccgattatttcccgtttctacaa
aagattgacccccaaggcattaggcggcgcaacacggttactttccg
gaaactgatcaacttgtttgggcgtatcatcgaccaaagattgaaag
tgagagaagcgagtggttctttgaaagatgatgatattttagacact
cttatcaacatgatggtggtggatcaggagaagaaagaggatcagct
tgacaaaaccataattgaacattttttactggatttattttcagcgg
ggactgaaacgacttcaaccacgttggagtgggcaatggctgagcta
gtaaaagcgccagagattatgtcaaaagcccgagcagagctagatca
agttataggcaaaggaaaccaagtgaaggaatcggacgtatctcgac
tcccttacttacaagccattgttaaagaaaccttccgcatgcaccct
acagctccattattgattcctcgcaaagccgacagtgacatcgaaat
ctccgactatatcatcccgaaggatgctcaggtgattgtcaatgtat
gggccattggtagagactcaagcacatgggaaaatcccgacaagttt
ataccggagaggtttttggacatcgatatagatgtcggaggccggga
ttttaagctcattccgttcggtgctggtcggagaatatgtcccggat
tcccattggcgatgcgaatgttgcacttgatgttggggtctttgctt
cactcgtttgattggaagttggaagatggggttagacctgatgctct
aaacatggatgaaaagtttggcctcaccttgcaaatggctcagcctt
tgcgagctatccccgtgccgacaaagcattag,
(SEQ ID NO 45)
caagtcacgacggttgtcatttcctccaccaccatggctaaagaagt
cctccaggcaaacagccaagtcgtctccagccggacaatcaccgacg
caagccgcgcccacagacacagcgattttagcatggttatgttgccc
gtatcccctctgtggcgaaaccttcggaaaataagcaactcacactt
gctttcctccaaggctcttgatggcaacatggagctgagaaacaaaa
aggtgcaagagctcctaaatgatgtccacaaaagcgtccaggccggg
gaggcggtggagatcgcgagcctttctttcagagctactctgaatct
cttgtccaccacatttttctccatggacatggcggatgacacaaatt
ccgtcactctaaaagagctcaaggaggctatgtcgcacatgatggaa
gagttggggaagcctaacttggccgattatttcccgtttctacaaaa
gattgacccccaaggcattaggcggcgcaacacggttactttccgga
aactgatcaacttgtttgggcgtatcatcgaccaaagattgaaagtg
agagaagcgagtggttctttgaaagatgatgatattttagacactct
tatcaacatgatggtggtggatcaggagaagaaagaggatcagcttg
acaaaaccataattgaacattttttactggatttattttcagcgggg
actgaaacgacttcaaccacgttggagtgggcaatggctgagctagt
aaaagcgccagagattatgtcaaaagcccgagcagagctagatcaag
ttataggcaaaggaaaccaagtgaaggaatcggacgtatctcgactc
ccttacttacaagccattgttaaagaaaccttccgcatgcaccctac
agctccattattgattcctcgcaaagccgacagtgacatcgaaatct
ccgactatatcatcccgaaggatgctcag,
(SEQ ID NO 69)
aaacctcgtcccatcatcggaagcctcttggagctcggcgaccaacc
ccacaggtccttggccaggctttccgagtcttacggcccgtttatgc
atttgaagctcggccaagtcacgacggttgtcatttcctccaccacc
atggctaaagaagtcctccaggcaaacagccaagtcgtctccagccg
gacaatcaccgacgcaagccgcgcccacagacacagcgattttagca
tggttatgttgcccgtatcccctctgtggcgaaaccttcggaaaata
agcaactcacacttgctttcctccaaggctcttgatggcaacatgga
gctgagaaacaaaaaggtgcaagagctcctaaatgatgtccacaaaa
gcgtccaggccggggaggcggtggagatcgcgagcctttctttcaga
gctactctgaatctcttgtccaccacatttttctccatggacatggc
ggatgacacaaattccgtcactctaaaagagctcaaggaggctatgt
cgcacatgatggaagagttggggaagcctaacttggccgattatttc
ccgtttctacaaaagattgacccccaaggcattaggcggcgcaacac
ggttactttccggaaactgatcaacttgtttgggcgtatcatcgacc
aaagattgaaagtgagagaagcgagtggttctttgaaagatgatgat
attttagacactcttatcaacatgatggtggtggatcaggagaagaa
agaggatcagcttgacaaaaccataattgaacattttttactggatt
tattttcagcggggactgaaacgacttcaaccacgttggagtgggca
atggctgagctagtaaaagcgccagagattatgtcaaaagcccgagc
agagctagatcaagttataggcaaaggaaaccaagtgaaggaatcgg
acgtatctcgactcccttacttacaagccattgttaaagaaaccttc
cgcatgcaccctacagctccattattgattcctcgcaaagccgacag
tgacatcgaaatctccgactatatcatcccgaaggatgctcaggtga
ttgtcaatgtatgggccattggtagagactcaagcacatgggaaaat
cccgacaagtttataccggagaggtttttggacatcgatatagatgt
cggaggccgggattttaagctcattccgttcggtgctggtcggagaa
tatgtcccggattcccattggcgatgcgaatgttgcacttgatgttg
gggtctttgcttcactcgtttgattggaagttggaagatggggttag
acctgatgctctaaacatggatgaaaagtttggcctcaccttgcaaa
tggctcagcctttgcgagctatccccgtgccgacaaagcat,
et
(SEQ ID 71)
aaacctcgtcccatcatcggaagcctcttggagctcggcgaccaacc
ccacaggtccttggccaggctttccgagtcttacggcccgtttatgc
atttgaagctcggccaagtcacgacggttgtcatttcctccaccacc
atggctaaagaagtcctccaggcaaacagccaagtcgtctccagccg
gacaatcaccgacgcaagccgcgcccacagacacagcgattttagca
tggttatgttgcccgtatcccctctgtggcgaaaccttcggaaaata
agcaactcacacttgctttcctccaaggctcttgatggcaacatgga
gctgagaaacaaaaaggtgcaagagctcctaaatgatgtccacaaaa
gcgtccaggccggggaggcggtggagatcgcgagcctttctttcaga
gctactctgaatctcttgtccaccacatttttctccatggacatggc
ggatgacacaaattccgtcactctaaaagagctcaaggaggctatgt
cgcacatgatggaagagttggggaagcctaacttggccgattatttc
ccgtttctacaaaagattgacccccaaggcattaggcggcgcaacac
ggttactttccggaaactgatcaacttgtttgggcgtatcatcgacc
aaagattgaaagtgagagaagcgagtggttctttgaaagatgatgat
attttagacactcttatcaacatgatggtggtggatcaggagaagaa
agaggatcagcttgacaaaaccataattgaacattttttactggatt
tattttcagcggggactgaaacgacttcaaccacgttggagtgggca
atggctgagctagtaaaagcgccagagattatgtcaaaagcccgagc
agagctagatcaagttataggcaaaggaaaccaagtgaaggaatcgg
acgtatctcgactcccttacttacaagccattgttaaagaaaccttc
cgcatgcaccctacagctccattattgattcctcgcaaagccgacag
tgacatcgaaatctccgactatatcatcccgaaggatgctcaggtga
ttgtcaatgtatgggccattggtagagactcaagcacatgggaaaat
cccgacaagtttataccggagaggtttttggacatcgatatagatgt
cggaggccgggattttaagctcattccgttcggtgctggtcggagaa
tatgtcccggattcccattggcgatgcgaatgttgcacttgatgttg
gggtctttgcttcactcgtttgattggaagttggaagatggggttag
acctgatgctctaaacatggatgaaaagtttggcctcaccttgcaaa
tggctcagcctttgcgagctatccccgtgccgacaaagcat
preferably with sequence SEQ ID NO 11 or SEQ ID
NO 71

For example, it may be a polynucleotide or nucleic acid coding for a fusion protein according to the invention, wherein the polynucleotide sequence or nucleic acid coding for a binding polypeptide is selected from the group comprising the nucleic acid of sequence:

(SEQ ID NO 15)
ccgggcggttctggtggcggtagcggcggtggcggttctggcggtgg
cggtagcggcggtggcggttctggcggtggcggtagcggcggtggcg
gttctggcggtggcggtagcggcggtggcggttctggtggcggtagc
ggcggttctccg.

For example, it may be a polynucleotide or nucleic acid coding for a fusion protein according to the invention wherein the polynucleotide sequence or nucleic acid coding for a polypeptide comprising the hydrophilic domain of a NADPH P450 reductase of cytochrome P450 of plant is selected from the group comprising the nucleic acid of sequence:

(SEQ ID NO 16)
atgacttctgctttgtatgcttccgatttgtttaagcagctcaagtc
aattatggggacagattcgttatccgacgatgttgtacttgtgattg
caacgacgtctttggcactagtagctggatttgtggtgttgttatgg
aagaaaacgacggcggatcggagcggggagctgaagcctttgatgat
ccctaagtctcttatggctaaggacgaggatgatgatttggatttgg
gatccgggaagactagagtctctatcttcttcggtacgcagactgga
acagctgagggatttgctaaggcattatccgaagaaatcaaagcgag
atatgaaaaagcagcagtcaaagtcattgacttggatgactatgctg
ccgatgatgaccagtatgaagagaaattgaagaaggaaactttggca
tttttctgtgttgctacttatggagatggagagcctactgacaatgc
tgccagattttacaaatggtttacggaggaaaatgaacgggatataa
agcttcaacaactagcatatggtgtgtttgctcttggtaatcgccaa
tatgaacattttaataagatcgggatagttcttgatgaagagttatg
taagaaaggtgcaaagcgtcttattgaagtcggtctaggagatgatg
atcagagcattgaggatgattttaatgcctggaaagaatcactatgg
tctgagctagacaagctcctcaaagacgaggatgataaaagtgtggc
aactccttatacagctgttattcctgaataccgggtggtgactcatg
atcctcggtttacaactcaaaaatcaatggaatcaaatgtggccaat
ggaaatactactattgacattcatcatccctgcagagttgatgttgc
tgtgcagaaggagcttcacacacatgaatctgatcggtcttgcattc
atctcgagttcgacatatccaggacgggtattacatatgaaacaggt
gaccatgtaggtgtatatgctgaaaatcatgttgaaatagttgaaga
agctggaaaattgcttggccactctttagatttagtattttccatac
atgctgacaaggaagatggctccccattggaaagcgcagtgccgcct
cctttccctggtccatgcacacttgggactggtttggcaagatacgc
agaccttttgaaccctcctcgaaagtctgcgttagttgccttggcgg
cctatgccactgaaccaagtgaagccgagaaacttaagcacctgaca
tcacctgatggaaaggatgagtactcacaatggattgttgcaagtca
gagaagtcttttagaggtgatggctgcttttccatctgcaaaacccc
cactaggtgtattttttgctgcaatagctcctcgtctacaacctcgt
tactactccatctcatcctcgccaagattggcgccaagtagagttca
tgttacatccgcactagtatatggtccaactcctactggtagaatcc
acaagggtgtgtgttctacgtggatgaagaatgcagttcctgcggag
aaaagtcatgaatgtagtggagccccaatctttattcgagcatctaa
tttcaagttaccatccaacccttcaactccaatcgttatggtgggac
ctgggactgggctggcaccttttagaggttttctgcaggaaaggatg
gcactaaaagaagatggagaagaactaggttcatctttgctcttctt
tgggtgtagaaatcgacagatggactttatatacgaggatgagctca
ataattttgttgatcaaggcgtaatatctgagctcatcatggcattc
tcccgtgaaggagctcagaaggagtatgttcaacataagatgatgga
gaaggcagcacaagtttgggatctaataaaggaagaaggatatctct
atgtatgcggtgatgctaagggcatggcgagggacgtccaccgaact
ctacacaccattgttcaggagcaggaaggtgtgagttcgtcagaggc
agaggctatagttaagaaacttcaaaccgaaggaagatacctcagag
atgtctggtga,
(SEQ ID NO 17)
actagagtctctatcttcttcggtacgcagactggaacagctgaggg
atttgctaaggcattatccgaagaaatcaaagcgagatatgaaaaag
cagcagtcaaagtcattgacttggatgactatgctgccgatgatgac
cagtatgaagagaaattgaagaaggaaactttggcatttttctgtgt
tgctacttatggagatggagagcctactgacaatgctgccagatttt
acaaatggtttacggaggaaaatgaacgggatataaagcttcaacaa
ctagcatatggtgtgtttgctcttggtaatcgccaatatgaacattt
taataagatcgggatagttcttgatgaagagttatgtaagaaaggtg
caaagcgtcttattgaagtcggtctaggagatgatgatcagagcatt
gaggatgattttaatgcctggaaagaatcactatggtctgagctaga
caagctcctcaaagacgaggatgataaaagtgtggcaactccttata
cagctgttattcctgaataccgggtggtgactcatgatcctcggttt
acaactcaaaaatcaatggaatcaaatgtggccaatggaaatactac
tattgacattcatcatccctgcagagttgatgttgctgtgcagaagg
agcttcacacacatgaatctgatcggtcttgcattcatctcgagttc
gacatatccaggacgggtattacatatgaaacaggtgaccatgtagg
tgtatatgctgaaaatcatgttgaaatagttgaagaagctggaaaat
tgcttggccactctttagatttagtattttccatacatgctgacaag
gaagatggctccccattggaaagcgcagtgccgcctcctttccctgg
tccatgcacacttgggactggtttggcaagatacgcagaccttttga
accctcctcgaaagtctgcgttagttgccttggcggcctatgccact
gaaccaagtgaagccgagaaacttaagcacctgacatcacctgatgg
aaaggatgagtactcacaatggattgttgcaagtcagagaagtcttt
tagaggtgatggctgcttttccatctgcaaaacccccactaggtgta
ttttttgctgcaatagctcctcgtctacaacctcgttactactccat
ctcatcctcgccaagattggcgccaagtagagttcatgttacatccg
cactagtatatggtccaactcctactggtagaatccacaagggtgtg
tgttctacgtggatgaagaatgcagttcctgcggagaaaagtcatga
atgtagtggagccccaatctttattcgagcatctaatttcaagttac
catccaacccttcaactccaatcgttatggtgggacctgggactggg
ctggcaccttttagaggttttctgcaggaaaggatggcactaaaaga
agatggagaagaactaggttcatctttgctcttctttgggtgtagaa
atcgacagatggactttatatacgaggatgagctcaataattttgtt
gatcaaggcgtaatatctgagctcatcatggcattctcccgtgaagg
agctcagaaggagtatgttcaacataagatgatggagaaggcagcac
aagtttgggatctaataaaggaagaaggatatctctatgtatgcggt
gatgctaagggcatggcgagggacgtccaccgaactctacacaccat
tgttcaggagcaggaaggtgtgagttcgtcagaggcagaggctatag
ttaagaaacttcaaaccgaaggaagatacctcagagatgtctgg,
preferably with sequence SEQ ID NO 17.

In the present, the terms “polynucleotide(s)”, “oligonucleotide(s)”, “nucleic acid(s)”, “polynucleic acid(s)” or any grammatical equivalent used herein refer to a polymeric form of nucleotides or nucleic acids of any length, whether they are ribonucleotides or deoxyribonucleotides. This term refers solely to the primary structure of the molecule. Thus, this term includes double and single stranded DNA, triplex DNA, as well as double and single stranded RNA. It also includes the modified forms, for example by methylation and/or capping, and the unmodified forms of the polynucleotide. The term also encompasses molecules which comprise unnatural or synthetic nucleotides as well as nucleotide analogs. The nucleic acid sequences and vectors disclosed or contemplated herein can be introduced into a cell, for example by transfection, transformation or transduction.

In the present, the nucleic acid or polynucleotide can be produced and/or obtained by any suitable method known to the person skilled in the art.

Another object of the invention relates to a vector comprising a nucleic acid coding for a fusion protein according to the invention. For example, this may be a vector comprising a polynucleotide or nucleic acid coding for a fusion protein successively comprising (i) at least one polypeptide for targeting, and anchoring to, the bacterial membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of a NADPH P450 reductase of P450 cytochrome of plant.

In the present, the vector can be any vector known to the person skilled in the art suitable for expressing a nucleic acid. For example, this may be any vector replicated in a low copy number in bacteria known to the person skilled in the art and/or commercially available. One example is the vector described in Rosano G L and Ceccarelli E A “Recombinant protein expression in Escherichia coli: advances and challenges” Front. Microbiol. 2014, 5:172. doi: 10.3389/fmicb.2014.00172 [15], for example replicated in about ten copies per bacteria. For example, this could be any of the vectors listed in the catalog https://blog.addgene.org/plasmid-101-origin-of-replication [4] or https://www.qiagen.com/cn/resources/faq?id=1f42840e-fbd7-4734-b0cd-e17372a9e5a4&lang=en [5]. It may be, for example, the expression vector described in document WO 83/004261 [7].

The vector can be any suitable plasmid known to the person skilled in the art and/or commercially available. For example, it may be a plasmid selected from the group comprising pACYC, pSC101, SuperCos, pWE15, pGEX, pColE1, pR-K, pAIDA1, preferably pAIDA1.

For example, this may be a modified plasmid, for example plasmid pAIDA1, comprising a tetracycline resistance gene and a cloning cassette comprising the T7 RNA polymerase promoter and the T7 RNA polymerase terminator. For example, this may be the plasmid pAIDA1, wherein the cmIA gene is replaced by the tetracycline resistance gene of sequence

(SEQ ID NO 18)
ttctcatgtttgacagcttatcatcgataagctttaatgcggtagtttatcacagttaaattgctaacgcagtcaggcaccgtgta
tgaaatctaacaatgcgctcatcgtcatcctcggcaccgtcaccctggatgctgtaggcataggcttggttatgccggtactg
ccgggcctcttgcgggatatcgtccattccgacagcatcgccagtcactatggcgtgctgctagcgctatatgcgttgatgca
atttctatgcgcacccgttctcggagcactgtccgaccgctttggccgccgcccagtcctgctcgcttcgctacttggagcca
ctatcgactacgcgatcatggcgaccacacccgtcctgtggatcctctacgccggacgcatcgtggccggcatcaccggc
gccacaggtgcggttgctggcgcctatatcgccgacatcaccgatggggaagatcgggctcgccacttcgggctcatgag
cgcttgtttcggcgtgggtatggtggcaggccccgtggccgggggactgttgggcgccatctccttgcatgcaccattccttg
cggcggcggtgctcaacggcctcaacctactactgggctgcttcctaatgcaggagtcgcataagggagagcgtcgacc
gatgcccttgagagccttcaacccagtcagctccttccggtgggcgcggggcatgactatcgtcgccgcacttatgactgtc
ttctttatcatgcaactcgtaggacaggtgccggcagcgctctgggtcattttcggcgaggaccgctttcgctggagcgcga
cgatgatcggcctgtcgcttgcggtattcggaatcttgcacgccctcgctcaagccttcgtcactggtcccgccaccaaacgt
ttcggcgagaagcaggccattatcgccggcatggcggccgacgcgctgggctacgtcttgctggcgttcgcgacgcgag
gctggatggccttccccattatgattcttctcgcttccggcggcatcgggatgcccgcgttgcaggccatgctgtccaggcag
gtagatgacgaccatcagggacagcttcaaggatcgctcgcggctcttaccagcctaacttcgatcattggaccgctgatc
gtcacggcgatttatgccgcctcggcgagcacatggaacgggttggcatggattgtaggcgccgccctataccttgtctgcc
tccccgcgttgcgtcgcggtgcatggagccgggccacctcgacctgaatggaagccggcggcacctcgctaacggattc
accactccaagaattggagccaatcaattcttgcggagaactgtgaatgcgcaaaccaacccttggcagaacatatccat
cgcgtccgccatctccagcagccgcacgcggcgcatctcgggcagcgt
and wherein the AIDA cassette
(SEQ ID NO 44)
taatacgactcactataggggaattgtgagcggataacaattcccctctagaaataattttgtttaactttaagaaggagatat
accatgggcaataaggcctacagtatcatttggagccactccagacaggcctggattgtggcctcagagttagccagagg
acatggttttgtccttgcaaaaaatacactgctggtattggcggttgtttccacaatcggaaatgcatttgcagtcgaccaccat
caccatcaccatctggaagcgctgttccagggtccgggtacccagaaacagcgtaccgagctcgaaaacctgtacttcc
agggtgaacagaaactgattagcgaagaagatctgtctagagtgaataacaatggaagcattgtcattaataacagcatt
ataaacgggaatattacgaatgatgctgacttaagttttggtacagcaaagctgctctctgctacagtgaatggtagtcttgtt
aataacaaaaatatcattcttaatcctacaaaagaaagtgcggccgctataggtaatactcttaccgtgtcaaattatactgg
gacaccgggaagtgttatttctcttggtggtgtgcttgaaggagataattcacttacggaccgtctggtggtgaaaggtaata
cctctggtcaaagtgacatcgtttatgtcaatgaagatggcagtggtggtcagacgagagatggtattaatattatttctgtag
agggaaattctgatgcagaattctctctgaagaaccgcgtagttgccggagcttatgattacacactgcagaaaggaaac
gagagtgggacagataataagggatggtatttaaccagtcatcttcccacatctgatacccggcaatacagaccggaga
acggaagttatgctaccaatatggcactggctaactcactgttcctcatggatttgaatgagcgtaagcaattcagggccat
gagtgataatacacagcctgagtctgcatccgtgtggatgaagatcactggaggaataagctctggtaagctgaatgacg
ggcaaaataaaacaacaaccaatcagtttatcaatcagctcgggggggatatttataaattccatgctgaacaactgggtg
attttaccttagggattatgggaggatacgcgaatgcaaaaggtaaaacgataaattacacgagcaacaaagctgccag
aaacacactggatggttattctgtcggggtatacggtacgtggtatcagaatggggaaaatgcaacagggctctttgctga
aacttggatgcaatataactggtttaatgcatcagtgaaaggtgacggactggaagaagaaaaatataatctgaatggttt
aaccgcttctgcaggtgggggatataacctgaatgtgcacacatggacatcacctgaaggaataacaggtgaattctggtt
acagcctcatttgcaggctgtctggatgggggttacaccggatacacatcaggaggataacggaacggtggtgcaggga
gcagggaaaaataatattcagacaaaagcaggtattcgtgcatcctggaaggtgaaaagcaccctggataaggatacc
gggcggaggttccgtccgtatatagaggcaaactggatccataacactcatgaatttggtgttaaaatgagtgatgacagc
cagttgttgtcaggtagccgaaatcagggagagataaagacaggtattgaaggggtgattactcaaaacttgtcagtgaat
ggcggagtcgcatatcaggcaggaggtcacgggagcaatgccatctccggagcactggggataaaatacagcttctga
taatgacagatccggctgctaacaaagcccgaaaggaagctgagttggctgctgccaccgctgagcaataactagcata
accccttggggcctctaaacgggtcttgaggggttttttg
is replaced by an expression cassette comprising the promoter sequence
(SEQ ID NO 19)
taatacgactcactata
and the terminator sequence
(SEQ ID NO 20)
tagcataaccccttggggcctctaaacgggtcttgaggggttttttg of the T7 RNA ploymerase gene.

The vector can be any suitable yeast artificial chromosome known to the person skilled in the art. It may involve, for example, a yeast artificial chromosome selected from the group comprising pYAC-RC, pYAC3(+).

The vector can be any suitable bacterial artificial chromosome known to the person skilled in the art. It may involve, for example, a bacterial artificial chromosome selected from the group comprising pUvBBAC, pCC1BAC, pBAC 108L.

The expression vector of fusion protein according to the invention may comprise the following functionally linked elements:
a) a promoter
b) a sequence coding for a fusion protein according to the invention
c) transcription termination signals.

In the present, “promoter” means a cis-acting DNA sequence located 5′ from the transcription initiation site of the sequence coding the polypeptide to which an RNA polymerase DNA sequence can bind and initiate correct transcription, and optionally comprising activators. For example, this may be any suitable promoter known to the person skilled in the art. For example, this may be a constitutive promoter, a viral promoter, or a bacterial promoter. For example, the T7 promoter, the T7 bacteriophage promoter, preferably the T7 promoter.

In the present, “transcription termination signals” means a DNA sequence adapted for transcription termination. It may be any transcription termination signal known to the person skilled in the art. It may be, for example, the T7 terminator with sequence tagcataaccccttggggcctctaaacgggtcttgaggggttttttg (SEQ ID NO 20).

The vector can be selected according to the selected host cell. The person skilled in the art, in view of his technical knowledges, will adapt the vector to the host cell.

The host cell can be any cell suitable for expression of a nucleic acid or vector according to the invention. It may be, for example, bacteria, for example gram-negative or gram-positive bacteria. It may be, for example, Escherichia coli, Pischia pastoris, Saccharomyces cerevisiae. Preferably, the host cell is Escherichia coli, even more preferably, the host cell can be the bacterial strain Escherichia coli BL21 (DE3) pLysE.

Advantageously, the inventors have demonstrated that expression of the fusion protein in host cells, for example bacterial cells, advantageously enables production of the fusion protein and localization on the surface of the host cell membrane. For example, when the host cell is a gram-negative bacterium, expression of the fusion protein advantageously enables production of the fusion protein and localization on the surface of the bacterium's outer membrane. In other words, the inventors have demonstrated that expression of the fusion protein in host cells advantageously enables production of the fusion protein, localization to the host cell surface, and advantageously anchor the fusion protein to the host cell via its membrane.

Another object of the present invention is a host cell comprising a nucleic acid according to the invention and/or a vector according to the invention.

The host cell can be as defined above. Preferably the host cell is a bacterial cell, for example a gram-negative bacterium or a gram-positive bacterium, preferably Escherichia coli, even more preferably the host cell can be the bacterial strain Escherichia coli (BL21 (DE3) pLysE).

Another object of the present invention is a process for producing a fusion protein according to the invention, comprising culturing a host cell according to the invention under conditions suitable for expression of the fusion protein.

The host cell culture according to the invention can be performed in any suitable culture medium known to the person skilled in the art. For example, this may be any rich culture medium known to the person skilled in the art. With this general knowledge, the person skilled in the art will adapt and/or choose the culture medium to suit the host cell. For example, when the host cell is a bacterial cell, the culture medium may be an SOC medium or a lysogeny broth (LB) medium, preferably an SOC medium.

The host cell culture according to the invention can be carried out at any temperature suitable for the host cell known to the person skilled in the art. For example, the culture can be carried out at a temperature of between 2° and 30° C., for example a temperature of 26° C.

The culture time can be any time suitable for the host cell that is known to the person skilled in the art. For example, the host cell culture time according to the invention can be between 24 and 72 hours, for example 53 hours.

Advantageously, the inventors have demonstrated that the fusion protein according to the invention is present on the surface of the host cell in which it is expressed and is a functional protein. In particular, the inventors have surprisingly demonstrated that the fusion protein exhibits biological activity, in particular the enzymatic activity of plant P450 cytochrome and the reductase activity of the NADPH P450 reductase of cytochrome P450 of plant. In other words, the fusion protein advantageously simultaneously possesses the biological activity of the plant P450 cytochrome and the NADPH P450 reductase of cytochrome P450 of plant. The inventors have also demonstrated in a surprising and unexpected way that the fusion protein according to the invention being a functional protein and exhibiting the biological activity of the plant P450 cytochrome and a reductase activity of the plant NADPH P450 reductase of cytochrome P450 of plant enables a bioconversion of substrates present in the culture medium.

The present invention also relates to a process for bioconverting a substrate with a fusion protein according to the invention comprising the steps of:
introducing the substrate into a culture medium comprising a host cell according to the invention,
incubating said culture medium for a time sufficient for bioconversion of said substrate by said fusion protein, and
optionally, recovering the metabolites resulting from bioconversion of said substrate.

The culture medium is as defined above.

The host cell is as defined above.

In the present, the substrate may be any suitable substrate known to the person skilled in the art. For example, it may be a compound or substrate, for example a natural or obtained by chemical synthesis or hemi-synthesis. For example, it may be any compound or substrate, for example a natural or obtained by chemical synthesis or hemi-synthesis, known to the person skilled in the art. For example, it may be cinnamic acid, or demethylsuberosin. It can also be any new compound that one wants to demonstrate is a substrate for a plant P450 cytochrome.

In the present, the concentration of substrate in the culture medium comprising a host cell can range from 100 μM to 250 μM, for example equal to 200 μM.

In the present, prior to the incubation step, the bioconversion process may further comprise a step of introducing nicotinamide adenine dinucleotide phosphate (NADPH) into the culture medium.

In the present, the concentration of nicotinamide adenine dinucleotide phosphate (NADPH) in the culture medium comprising a host cell can be from 200 μM to 450 μM, for example equal to 400 μM.

The step of incubating the host cell culture according to the invention can be carried out at any temperature suitable for the host cell known to the person skilled in the art. For example, the incubation can be carried out at a temperature from 15 to 30° C., for example a temperature of 20° C.

The incubation time can be any time suitable for the host cell that is known to the person skilled in the art. For example, the host cell incubation time according to the invention can be from 30 minutes to 72 hours, for example 1 hour. In the present, the recovery of the metabolites can be performed by any suitable process known to the person skilled in the art. This may be a technique selected from ultrafiltration, membrane or gel filtration, ion exchange, elution on hydroxyapatite, separation by hydrophobic interactions, chromatography, e.g. liquid chromatography, or any other known means.

In the present, the obtained metabolites can be any plant metabolite. For example, they may be metabolites belonging to the polyphenol, alkaloid, and/or terpene family. Advantageously, it may be metabolites belonging to the polyphenol family.

The obtained metabolites can give access to new products which can be food, cosmetic, pharmaceutical, and parapharmaceutical actives for use in the agri-food, cosmetic, pharmaceutical, and parapharmaceutical fields. These new products may also be active or non-active, but their neutrality and/or stability make them highly attractive for use in each of these fields.

Other advantages will become more apparent in light of the following examples, given by way of non-limiting illustration, with reference to the appended figures.

### BRIEF DESCRIPTION OF THE FIGURES

FIG. 1 shows a schematic diagram of the fusion protein comprising in succession a polypeptide for targeting, and anchoring to, the outer membrane (C), a polypeptide comprising the hydrophilic domain of a plant P450 cytochrome (B), a binding polypeptide comprising at least 47 amino acids (L) and a polypeptide comprising the hydrophilic domain of a NADPH P450 reductase of cytochrome P450 of plant (A), where M corresponds to the bacterial membrane.

FIG. 2 represents a schematic representation of the plasmid pAIDA1-TetR-lacIQ.

FIG. 3 represents a schematic representation of the plasmid pAIDA1-T7.

FIG. 4 represents a schematic representation of the plasmid pAIDA1-T7

FIG. 5 represents a schematic representation of the plasmid pAIDA1-T7-CYP73A1-ATR1.

FIG. 6 represents a schematic diagram of the various components of an example of a fusion protein according to the invention. In the figure, AIDA1-beta-barrel AIDA1-β1 and AIDA1 linker represent, respectively, a polypeptide for targeting, and anchoring to, the outer membrane, a binding polypeptide, P450 73A1 a polypeptide comprising the hydrophilic domain of a plant P450 cytochrome (P450 CYP73A1), flexible linker: a binding polypeptide, and ATR1:P450 Reductase a polypeptide comprising the hydrophilic domain of a NADPH P450 reductase of P450 cytochrome of plant.

FIGS. 7A-7B represents chromatograms corresponding to high-performance liquid chromatography (HPLC) analysis of culture medium from a bioconversion experiment in the presence of cinnamic acid. The abscissa corresponds to the elution time in minutes and the ordinate corresponds to arbitrary units and allow to show the efficiency of metabolization. FIG. 7A represents a diagram concerning the metabolism of cinnamic acid by bioconversion. In this figure, FIG. 7A shows the chromatograms obtained after culture of the E. coli BL21 (DE3) pLysE transformed with the plasmid pAIDA1-T7-CYP73A1-ATR1 and FIG. 7B represents the diagram after culture of the E. coli BL21 (DE3) pLysE transformed with plasmid pAIDA1-T7 in FIG. 4. In FIG. 7A shows the appearance of para-coumaric acid, the metabolism product of cinnamic acid. In FIG. 7B, cinnamic acid is not metabolized.

FIG. 8 represents a peptide sequence alignment performed with the Basic Local Alignment Search Tool (BLAST). The sequences correspond to P450 cytochromes CYP76F112 and P450 CYP73A1

FIG. 9 represents the chemical reaction corresponding to the transformation of cinnamic acid into para-coumaric acid by cytochrome P450 CYP73A1 (CYP73A1) and the chemical reaction corresponding to the transformation of demethylsuberosin into Marmesin by cytochrome P450 CYP76F112 (CYP76F112).

### EXAMPLES

### Example 1: Process for Preparing a Fusion Protein, and Biotransformation Process Using Said Protein

1. Construction of a Generic Expression Plasmid pAIDA-T7

The basis of the expression plasmid is a commercial plasmid pAIDA1 (https://www.addgene.org/79180/[11]), which is a low-copy plasmid.

### Replacement of the Selection Gene

The pAIDA1 plasmid was first modified by replacing the gene conferring chloramphenicol resistance with a gene conferring tetracycline resistance cloned from the pBR322 plasmid marketed by Fisher Scientific (https://www.fishersci.fr/shop/products/fermentas-pbr322-dna/10191220 [11]). To this end, plasmid pAIDA1 was used as the template, which was amplified by polymerase chain reaction (PCR) using the enzyme PrimeSTAR Max polymerase marketed by Takara Bio Inc, according to the procedure Protocol 1: PrimeSTAR Max polymerase Protocol as described in (https://www.takarabio.com/documents/User%20Manual/R045A_e.v2102 Da.pdf [6]). Amplification was performed using primers LAEWXpr17-lacIQ (tggcgacaccatcgaatggtgc (SEQ ID NO: 21) and LAEWXpr02: Reverse: tttagcttccttagctcctg (SEQ ID NO: 22). This amplification enabled the plasmid to be copied in its entirety, with the exception of the chloramphenicol resistance gene. The same process was used to amplify the sequence coding for the tetracycline resistance gene. Amplification was performed this time from plasmid pBR322 using primers LAEWXpr03 (gctaaggaagctaaaatgaaatctaacaatgcgct (SEQ ID NO 41)) and LAEWXpr04 (tcgatggtgtcgccacgctgcccgagatgc (SEQ ID NO 42)). The two PCR products obtained, that is, the pAIDA1 plasmid without the chloramphenicol resistance gene and the sequence coding for the tetracycline resistance gene, were fused using the In-Fusion kit marketed by Takara Bio Inc, according to the Protocol 2: In-Fusion Protocol described in the document https://www.takarabio.com/documents/User%20Manual/In/In-Fusion%20Snap%20Assembly%20User%20Manual_071320.pdf [16]. The recombinant plasmid obtained was introduced into chemocompetent Escherichia coli TOP10 bacteria marketed by Life Technologies Corporation according to the TOP10 transformation protocol described in https://assets.thermofisher.com/TFS-Assets/LSG/manuals/oneshottop10_man.pdf [17]. The transformed bacteria were plated on an LB (lysogenic broth) culture medium (10 g peptone, 5 g yeast extract, 5 g NaCl) containing tetracycline. The insertion of the gene coding for tetracycline resistance was verified by PCR using primers LAEWXpr17 (SEQ ID NO 21) and LAEWXpr02 (SEQ ID NO 22).

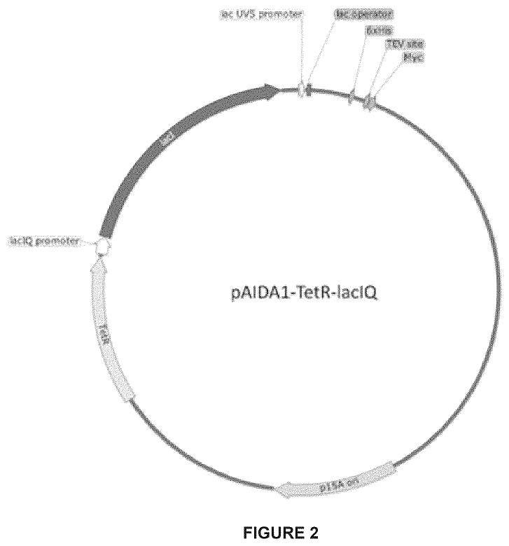

The recombinant plasmid pAIDA1-TetR-lacI^{Q}, shown in FIG. 2, was used to construct the other plasmids. To simplify the nomenclature, the plasmid is also referred to as pAIDA1. This plasmid was amplified and purified from a positive colony using the Protocol 3 plasmid purification protocol described in the document https://www.mn-net.com/media/pdf/45/51/02/Instruction-NucleoSpin-Plasmid.pdf [18].

### A) Promoter and Terminator

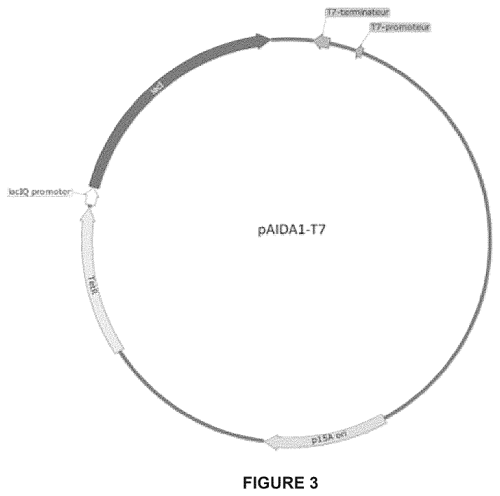

The promoter and terminator of the AIDA cassette of plasmid pAIDA1 were replaced by two cloning cassettes featuring the promoter and terminator of T7 RNA Polymerase. To carry out this step, various cloning techniques were used
(i) pAIDA1 was amplified by PCR using primers LAEWXpr30 (tcatcatcatgcctaatgagtgagaattcc (SEQ ID NO 23)) and LAEWXpr31 (ttggtgcgcaaactattaactgg (SEQ ID NO 24)). This amplification step involves the DNA fragment contained between the pAIDA1 promoter and the pAIDA1 terminator. The amplification product therefore corresponded to the plasmid without promoter and terminator
(ii) In parallel, the T7 terminator was amplified by PCR from plasmid pET28b-2 using primers LAEWXpr32-1-fr (ctcattaggcatgatgatgaaaggaagggaagaaagcgaaagg (SEQ ID NO 25)) and LAEWXpr32-2-rv (agtactcctaggactagtggtaccagatccggctgctaacaaagc (SEQ ID NO 26)). Finally, the T7 promoter was amplified by PCR from plasmid pET28b-2 using primers LAEWXpr33-1-rv (gttaatagtttgcgcaccaaatcggtgatgtcggcgatatagg (SEQ ID NO 27)) and LAEWXpr33-2-fr (ggtaccactagtcctaggagtactatggctgctgcccatggtata (SEQ ID NO 28)) according to Protocol 1 as mentioned above,
(iii) The three fragments were fused using the In-Fusion kit according to Protocol 2 as described above. This ligation product was used to generate the pAIDA1-T7 plasmid shown in FIG. 3.

### b) Inserting the AIDA1 Cassette

The AIDA sequence of the original pAIDA1 plasmid was amplified by PCR using primers LAEWXpr36-AIDA-fr (aactttaagaaggagatataccatgggcaataaggcctacagtatcatttgg (SEQ ID NO 29)) and LAEWXpr37-AIDA-rv (tttgttagcagccggatctgtcattatcagaagctgtattttatc (SEQ ID NO 30)) according to Protocol 1 as mentioned above.

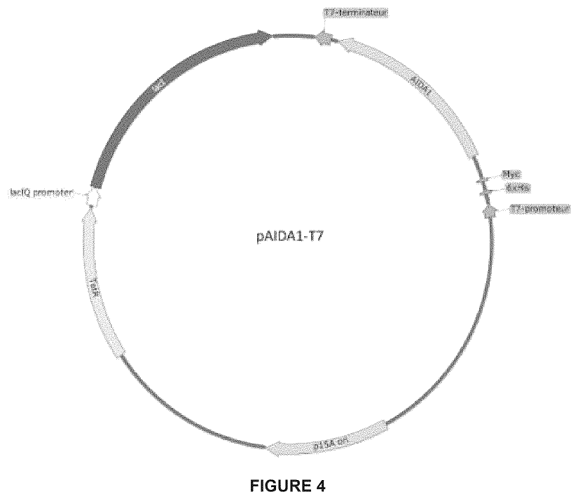

In parallel, plasmid pAIDA1-T7 was digested with NcoI and KpnI restriction enzymes using the Protocol 4 digestion protocol as described in http://assets.thermofisher.com/TFS-Assets/BID/Reference-Materials/fastdigest-restriction-enzymes-labaid.pdf [19]. The linearized pAIDA1-T7-NcoI-KpnI plasmid and the AIDA1 amplicon were fused by In-Fusion according to Protocol 2 above. The generic recombinant plasmid pAIDA1-T7 complete with AIDA1 was amplified by bacterial transformation. The obtained generic complete plasmid pAIDA1-T7 with AIDA1 is shown in FIG. 4.

### 2) Construction of a Recombinant Plasmid for Expression of a P450-ATR Fusion Protein

a) Plasmid pAIDA1-T7 was digested with the restriction enzymes KpnI and SacI according to Protocol 4 as described above.

b) The sequence coding for the extra-membrane portion of Arabidopsis thaliana NADPH P450 reductase 1 (ATR1) as described in Urban et al, (1997) J Biol Chem 272(31):19176-86 [20]) was amplified by PCR from a plasmid (pCR8_ATR1) using primers LAEWXpr05 Forward (agcgctgttccagggtccgggtaccactagagtctctatcttc (SEQ ID NO 31)) and LAEWXpr06 Reverse (ccgccaccagaaccgcccggccagacatctgaggtatc (SEQ ID NO 32)) according to Protocol 1 as mentioned above).

c) The linker (rich in GC pairs) was amplified from a synthetic sequence using the PrimeSTAR GXL DNA Polymerase enzyme marketed by Takara Bio Inc. using primers LAEWX15-FlexL-fr (ccgggcggttggtggcgg (SEQ ID NO 33)) and LAEWX16-FlexL-rvs (cggagaaccgccgctaccgc (SEQ ID NO 34)) according to Protocol 5 described in PrimeSTAR GXL DNA Polymerase Manual, https://www.takara.co.kr/file/manual/pdf/R050A_e.v1906 Da.pdf [21]).

d) The DNA sequence coding for the extra-membrane part of P450, CYP73A1 (Urban et al, (1997) J Biol Chem 272(31):19176-86 [20]) was amplified by PCR from a plasmid pYeDP60-CYP73A1 plasmid using primers LAEWXpr07 Forward (gcggtagcggcggttctccgcctggcccaatcccggttcc (SEQ ID NO 35)) and LAEWXpr08 Reverse (gaagtacaggttttcgagctcaaatgacctaggtttagc (SEQ ID NO 36)) according to Protocol 1 above.

e) The 4 DNA fragments generated by PCR amplification, namely SEQ ID NO 17, SEQ ID NO 15, SEQ ID NO 11 and plasmid pAIDA1-T7 were digested with the restriction enzymes KpnI and SacI according to Protocol 4 as mentioned above. The obtained plasmid sequence corresponds to the sequence:

(SEQ ID NO 43)
aaggaagggaagaaagcgaaaggagcgggcgctagggcgctggcaagtgtagcggtcacgctgcgcgtaaccacc
acacccgccgcgcttaatgcgccgctacagggcgcgtcccattcgccaatccggatatagttcctcctttcagcaaaaaac
ccctcaagacccgtttagaggccccaaggggttatgctagttattgctcagcggtggcagcagccaactcagcttcctttcg
ggctttgttagcagccggatctgtcattatcagaagctgtattttatccccagtgctccggagatggcattgctcccgtgacctc
ctgcctgatatgcgactccgccattcactgacaagttttgagtaatcaccccttcaatacctgtctttatctctccctgatttcggc
tacctgacaacaactggctgtcatcactcattttaacaccaaattcatgagtgttatggatccagtttgcctctatatacggacg
gaacctccgcccggtatccttatccagggtgcttttcaccttccaggatgcacgaatacctgcttttgtctgaatattatttttccct
gctccctgcaccaccgttccgttatcctcctgatgtgtatccggtgtaacccccatccagacagcctgcaaatgaggctgtaa
ccagaattcacctgttattccttcaggtgatgtccatgtgtgcacattcaggttatatcccccacctgcagaagcggttaaacc
attcagattatatttttcttcttccagtccgtcacctttcactgatgcattaaaccagttatattgcatccaagtttcagcaaagagc
cctgttgcattttccccattctgataccacgtaccgtataccccgacagaataaccatccagtgtgtttctggcagctttgttgct
cgtgtaatttatcgttttaccttttgcattcgcgtatcctcccataatccctaaggtaaaatcacccagttgttcagcatggaattta
taaatatcccccccgagctgattgataaactgattggttgttgttttattttgcccgtcattcagcttaccagagcttattcctccag
tgatcttcatccacacggatgcagactcaggctgtgtattatcactcatggccctgaattgcttacgctcattcaaatccatga
ggaacagtgagttagccagtgccatattggtagcataacttccgttctccggtctgtattgccgggtatcagatgtgggaaga
tgactggttaaataccatcccttattatctgtcccactctcgtttcctttctgcagtgtgtaatcataagctccggcaactacgcgg
ttcttcagagagaattctgcatcagaatttccctctacagaaataatattaataccatctctcgtctgaccaccactgccatcttc
attgacataaacgatgtcactttgaccagaggtattacctttcaccaccagacggtccgtaagtgaattatctccttcaagca
caccaccaagagaaataacacttcccggtgtcccagtataatttgacacggtaagagtattacctatagcggccgcacttt
cttttgtaggattaagaatgatatttttgttattaacaagactaccattcactgtagcagagagcagctttgctgtaccaaaactt
aagtcagcatcattcgtaatattcccgtttataatgctgttattaatgacaatgcttccattgttattcactctagacagatcttcttc
gctaatcagtttctgttcaccctggaagtacaggttttcgagctccggaccctggaacagcgcttccagatggtgatggtgat
ggtggtcgactgcaaatgcatttccgattgtggaaacaaccgccaataccagcagtgtattttttgcaaggacaaaaccat
gtcctctggctaactctgaggccacaatccaggcctgtctggagtggctccaaatgatactgtaggccttattgcccatggta
tatctccttcttaaagttaaacaaaattatttctagaggggaattgttatccgctcacaattcccctatagtgagtcgtattaatttc
gcgggatcgagatctcgatcctctacgccggacgcatcgtggccggcatcaccggcgccacaggtgcggttgctggcgc
ctatatcgccgacatcaccgatttggtgcgcaaactattaactggcgaactacttactctagcttcccggcaacaattaatag
actggatggaggcggataaagttgcaggaccacttctgcgctcggcccttccggctggctggtttattgctgataaatctgga
gccggtgagcgtgggtctcgcggtatcattgcagcactggggccagatggtaagccctcccgtatcgtagttatctacacg
acggggagtcaggcaactatggatgaacgaaatagacagatcgctgagataggtgcctcactgattaagcattggtaact
gtcagaccaagtttactcatatatactttagattgatttaaaacttcatttttaatttaaaaggatctaggtgaagatcctttttgata
atctcatgaccaaaatcccttaacgtgagttttcgttccactgagcgtcagaccccttaataagatgatcttcttgagatcgtttt
ggtctgcgcgtaatctcttgctctgaaaacgaaaaaaccgccttgcagggcggtttttcgaaggttctctgagctaccaactc
tttgaaccgaggtaactggcttggaggagcgcagtcaccaaaacttgtcctttcagtttagccttaaccggcgcatgacttca
agactaactcctctaaatcaattaccagtggctgctgccagtggtgcttttgcatgtctttccgggttggactcaagacgatagt
taccggataaggcgcagcggtcggactgaacggggggttcgtgcatacagtccagcttggagcgaactgcctacccgg
aactgagtgtcaggcgtggaatgagacaaacgcggccataacagcggaatgacaccggtaaaccgaaaggcagga
acaggagagcgcacgagggagccgccagggggaaacgcctggtatctttatagtcctgtcgggtttcgccaccactgatt
tgagcgtcagatttcgtgatgcttgtcaggggggcggagcctatggaaaaacggctttgccgcggccctctcacttccctgtt
aagtatcttcctggcatcttccaggaaatctccgccccgttcgtaagccatttccgctcgccgcagtcgaacgaccgagcgt
agcgagtcagtgagcgaggaagcggaatatatcctgtatcacatattctgctgacgcaccggtgcagccttttttctcctgcc
acatgaagcacttcactgacaccctcatcagtgccaacatagtaagccagtatacactccgctagcgctgaggtctgcctc
gtgaagaaggtgttgctgactcataccaggcctgaatcgccccatcatccagccagaaagtgagggagccacggttgat
gagagctttgttgtaggtggaccagttggtgattttgaacttttgctttgccacggaacggtctgcgttgtcgggaagatgcgtg
atctgatccttcaactcagcaaaagttcgatttattcaacaaagccacgttgtgtctcaaaatctctgatgttacattgcacaag
ataaaaatatatcatcatgaacaataaaactgtctgcttacataaacagtaatacaaggggtgttatgagccatattcaacg
ggaaacgtcttgctcgagtatccgctcatgagattatcaaaaaggatcttcacctagatccttttgtaagttctcatgtttgaca
gcttatcatcgataagctttaatgcggtagtttatcacagttaaattgctaacgcagtcaggcaccgtgtatgaaatctaacaa
tgcgctcatcgtcatcctcggcaccgtcaccctggatgctgtaggcataggcttggttatgccggtactgccgggcctcttgc
gggatatcgtccattccgacagcatcgccagtcactatggcgtgctgctagcgctatatgcgttgatgcaatttctatgcgca
cccgttctcggagcactgtccgaccgctttggccgccgcccagtcctgctcgcttcgctacttggagccactatcgactacgc
gatcatggcgaccacacccgtcctgtggatcctctacgccggacgcatcgtggccggcatcaccggcgccacaggtgcg
gttgctggcgcctatatcgccgacatcaccgatggggaagatcgggctcgccacttcgggctcatgagcgcttgtttcggcg
tgggtatggtggcaggccccgtggccgggggactgttgggcgccatctccttgcatgcaccattccttgcggcggcggtgct
caacggcctcaacctactactgggctgcttcctaatgcaggagtcgcataagggagagcgtcgaccgatgcccttgaga
gccttcaacccagtcagctccttccggtgggcgcggggcatgactatcgtcgccgcacttatgactgtcttctttatcatgcaa
ctcgtaggacaggtgccggcagcgctctgggtcattttcggcgaggaccgctttcgctggagcgcgacgatgatcggcct
gtcgcttgcggtattcggaatcttgcacgccctcgctcaagccttcgtcactggtcccgccaccaaacgtttcggcgagaag
caggccattatcgccggcatggcggccgacgcgctgggctacgtcttgctggcgttcgcgacgcgaggctggatggcctt
ccccattatgattcttctcgcttccggcggcatcgggatgcccgcgttgcaggccatgctgtccaggcaggtagatgacgac
catcagggacagcttcaaggatcgctcgcggctcttaccagcctaacttcgatcattggaccgctgatcgtcacggcgattt
atgccgcctcggcgagcacatggaacgggttggcatggattgtaggcgccgccctataccttgtctgcctccccgcgttgc
gtcgcggtgcatggagccgggccacctcgacctgaatggaagccggggcacctcgctaacggattcaccactccaag
aattggagccaatcaattcttgcggagaactgtgaatgcgcaaaccaacccttggcagaacatatccatcgcgtccgcca
tctccagcagccgcacgcggcgcatctcgggcagcgtggcgacaccatcgaatggtgcaaaacctttcgcggtatggca
tgatagcgcccggaagagagtcaattcagggtggtgaatgtgaaaccagtaacgttatacgatgtcgcagagtatgccgg
tgtctcttatcagaccgtttcccgcgtggtgaaccaggccagccacgtttctgcgaaaacgcgggaaaaagtggaagcgg
cgatggcggagctgaattacattcccaaccgcgtggcacaacaactggcgggcaaacagtcgttgctgattggcgttgcc
acctccagtctggccctgcacgcgccgtcgcaaattgtcgcggcgattaaatctcgcgccgatcaactgggtgccagcgt
ggtggtgtcgatggtagaacgaagcggcgtcgaagcctgtaaagcggcggtgcacaatcttctcgcgcaacgcgtcagt
gggctgatcattaactatccgctggatgaccaggatgccattgctgtggaagctgcctgcactaatgttccggcgttatttcttg
atgtctctgaccagacacccatcaacagtattattttctcccatgaagacggtacgcgactgggcgtggagcatctggtcgc
attgggtcaccagcaaatcgcgctgttagcgggcccattaagttctgtctcggcgcgtctgcgtctggctggctggcataaat
atctcactcgcaatcaaattcagccgatagcggaacgggaaggcgactggagtgccatgtccggttttcaacaaaccatg
caaatgctgaatgagggcatcgttcccactgcgatgctggttgccaacgatcagatggcgctgggcgcaatgcgcgccat
taccgagtccgggctgcgcgttggtgcggatatctcggtagtgggatacgacgataccgaagacagctcatgttatatccc
gccgttaaccaccatcaaacaggattttcgcctgctggggcaaaccagcgtggaccgcttgctgcaactctctcagggcc
aggcggtgaagggcaatcagctgttgcccgtctcactggtgaaaagaaaaaccaccctggcgcccaatacgcaaacc
gcctctccccgcgcgttggccgattcattaatgcagctggcacgacaggtttcccgactggaaagcgggcaagtgagtgg
ataaccgtattaccgcctttgagtgagctgataccgggaattctcactcattaggcatgatgatga.

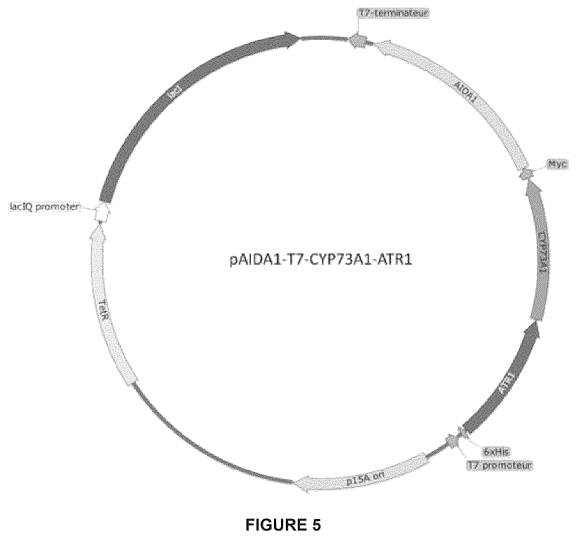

Next, the sequences SEQ ID NO 17, SEQ ID NO 15, SEQ ID NO 11 were fused by In-Fusion according to Protocol 2 mentioned above. The ligation product was introduced into E. coli bacteria and directly cultured without going through a selection phase on a selective medium. The presence of plasmid components was verified by PCR using primers LAEWXpr35-forward-T7 (tccatccagtctattaattgttgc (SEQ ID NO 37)), LAEWXpr22-ATR1-rv (ccagacatctctgaggtatcttcc (SEQ ID NO 38)), LAEWXpr24-2kbATR1-fr (ggagcaggaaggtgtgagttcgtc (SEQ ID NO 39)) and LAEWXpr25-P540-rv (accctggaagtacaggttttcg (SEQ ID NO 40)). Steps a to e allow to the construction of a pAIDA1-T7-CYP73A1-ATR1 plasmid shown in FIG. 5.

### 3) Fusion Protein

The resulting fusion protein contains from the N-terminus to the C-terminus:
a 6-Histidine tag, enabling protein immunodetection with anti-Histidine antibodies
a cleavage sequence to remove the tag,
a c-MYC sequence, enabling protein immunodetection with anti-c-MYC antibodies
an AIDA1 binding peptide
an outer membrane anchoring and addressing polypeptide: the AIDA1 beta Barel anchoring sequence, that is, SEQ ID NO 1
a polypeptide sequence of cytochrome P450 CYP73A1 (cinnamate hydroxylase from Helianthus tuberosus, NCBI ID: Sequence ID: Q04468.1, UniProtKB/Swiss-Prot: Q04468.1) wherein the membrane anchor has been removed from the coding sequence, that is, the sequence SEQ ID NO 3),
a 51 amino-acid binding peptide PGGSGGGSGGGGSGGGGSGGGGSGGGGSGGGGSGGGGSGGGGSGGGSGGSP (SEQ ID NO 6), and
a polypeptide sequence of Arabidopsis NADPH P450 reductase (Arabidopsis thaliana P450 reductase 1 (ATR1), mRNA-Sequence ID: NM_118585.4, NCBI Reference Sequence: NM_118585.4) wherein the membrane anchor has been removed, namely the sequence SEQ ID NO 7.

The coding nucleic sequences corresponding to the different elements of the fusion protein described above were used and assembled successively from 5′ to 3′. In particular, it involved:
the sequence SEQ ID NO 44 coding for an AIDA1 linker,
the sequence SEQ ID NO 10 coding for a polypeptide for targeting and anchoring to the external membrane: the beta barrel anchoring sequence
the sequence SEQ ID NO 11 coding for cytochrome P450 CYP73A1 cinnamate hydroxylase from Helianthus tuberosus, NCBI ID: Sequence ID: Q04468.1, UniProtKB/Swiss-Prot: Q04468.1)
the sequence SEQ ID NO 15 coding for a 51 amino-acid binding peptide, and
the sequence SEQ ID NO 17 coding for Arabidopsis NADPH P450 reductase (Arabidopsis thaliana P450 reductase 1 (ATR1), mRNA-Sequence ID: NM_118585.4, NCBI Reference Sequence: NM_118585.4)

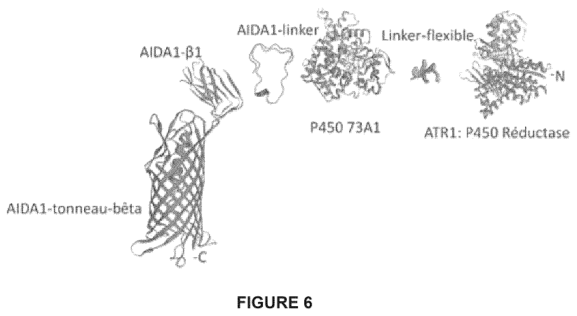

FIG. 6 is a schematic representation of the resulting fusion protein.

4) Expression of Recombinant Proteins in E. coli Bacteria

### A) The Expression System Used

The pAIDA1 plasmid and recombinant plasmids containing the genes coding for the fusion protein were introduced into E. coli BL21 (DE3) plysE” bacteria (Novagen's® pET Systems). BL21 (DE3) pLysE bacteria are adapted for the production of proteins under the control of the T7 promoter. BL21 (DE3) pLysE bacteria carry the lambda DE3 lysogen and contain the pLysE plasmid, which constitutively expresses the T7 lysozyme. The T7 lysozyme reduces basal expression of target genes by inhibiting T7 RNA polymerase. The BL21 (DE3) pLysE strain therefore provides tighter control of T7 RNA polymerase.

### B) Preparation of an Example Fusion Protein

The fusion protein was expressed from the plasmid whose construction was described above. Once the bacteria have been transformed by the ligation product, there is no selection step on solid medium. Transformed bacteria were immediately cultured in 50 mL SOC medium (Dextrose, 3.603 g/L, KCl, 0.186 g/L, MgSO4, 4.8 g/L, Tryptone, 20 g/L, Yeast extract 5 g/L) comprising Tetracycline (20 μg/mL) and Chloramphenicol (20 μg/mL), in a sterile 250 mL Erlenmeyer flask. The culture was carried out for 53 h at 26° C. with stirring of 180 revolutions per minute. The cultures were then cooled on ice for 10 minutes. The optical density of the culture was adjusted to D0 550 nm=0.3 by dilution in SOC medium (4° C.). The volume of culture medium was measured and antibiotics were re-added to a final concentration of 20 μg/mL Tetracycline and 20 μg/mL Chloramphenicol. Fusion protein production was induced by adding isopropyl β-D-1-thiogalactopyranoside (IPTG) at a concentration of 20 μM final for 24 h at 7° C., under 180 rpm agitation. After 24 h, optical density was adjusted to 0.3 by dilution in SOC medium (4° C.). Bacteria present in 1.5 mL were harvested by two successive low-speed centrifugations (4° C., 20 min, 1000×g and 4° C., 10 min, 4000×g).

Bacteria were then suspended in KPi buffer (88 mM with additives of KCl 1 mM, MgSO₄ 4 mM, glycerol 5% v/v, glucose 5% w/v, at 4° C. (products ordered from Sigma Aldrich)).

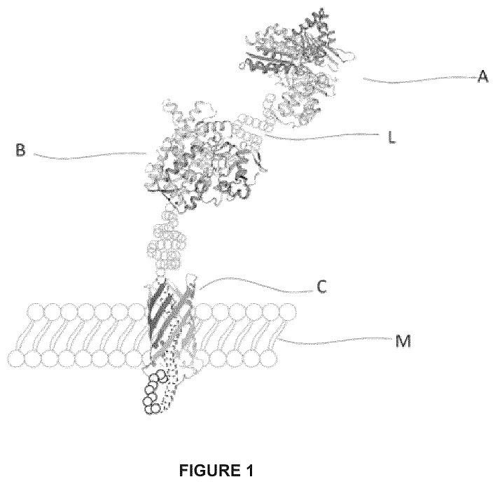

FIG. 1 is a schematic representation of the resulting fusion protein, said fusion protein being anchored in the bacterial membrane.

### 5) Bioconversion

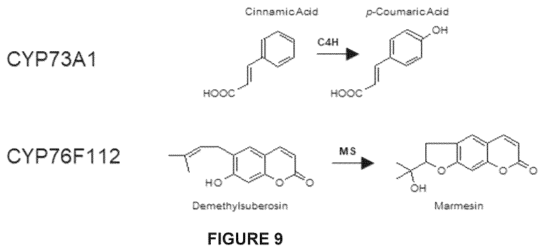

A study of the bioconversion of cinnamic acid was carried out. FIG. 9 (CYP73A1) shows the corresponding bioconversion reaction. For this purpose, cinnamic acid (200 μM) as substrate and nicotinamide adenine dinucleotide phosphate (NADPH) (400 μM) were added to the medium comprising the re-suspended E. coli bacteria obtained in point 4 above.

The bioconversion process was carried out at 20° C. for 1 h, with stirring at 180 rpm. The reaction was stopped by extraction with 1 volume of Ethyl Acetate. The media were vortexed for 1 min followed by centrifugation at 10,000×g to separate the organic and aqueous phases. The upper organic phase was recovered and evaporated by Vivaspin. The powder obtained was suspended in 150 μL of methanol. The extract thus obtained was analyzed by ultra-high-performance liquid chromatography coupled to a mass spectrum (UHPLC/MS/MS)

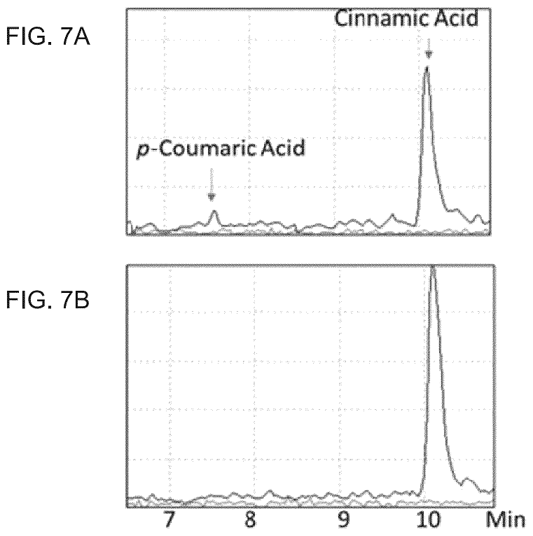

The obtained results are represented in FIGS. 7A-7B. FIG. 7A shows a chromatogram obtained at 300 nm, showing 2 peaks. The majority peak corresponds to the substrate, that is, cinnamate. The minority peak corresponds to p-coumarate or p-coumaric acid formed by bioconversion in the culture medium. This chromatogram was obtained from the culture medium wherein the recombinant bacteria transformed with the pAIDA1-T7-CYP73A1-ATR1 plasmids according to point 4 above were cultivated. The chromatogram in FIG. 7B corresponds to an analysis of the culture medium wherein the recombinant bacteria transformed with plasmid pAIDA1-T7 in FIG. 4. This plasmid cannot produce fusion protein and represents a negative control. Chromatogram analysis indicates the presence of cinnamate, which has been added to the culture medium. No metabolism of p-coumarate has been demonstrated.

This example therefore clearly demonstrates that an example fusion protein successively comprising (i) at least one polypeptide for targeting, and anchoring to, the bacterial membrane, advantageously to the external membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of a plant NADPH P450 reductase of cytochrome P450 of plant, enables the bioconversion of the substrate. This example also clearly demonstrates that an example of a fusion protein according to the invention can be expressed on the surface of a cell, in particular a bacterial cell, and can advantageously be used in a substrate bioconversion process.

This example also clearly demonstrates that an example of a fusion protein according to the invention can be expressed on the surface of a cell, in particular a bacterial cell, and can advantageously enable bioconversion of substrates in the culture medium of said cell.

### Example 2: Fusion Protein and Bioconversion

In this example, the cytochrome P450 is cytochrome CYP76F112 (marmesin synthase, Ficus carica cytochrome P450 CYP76F112 mRNA, complete cds Sequence ID: MW348922.1, GenBank: MW348922.1).

In this example, the process for obtaining the fusion protein is identical to the process described in Example 1, with the following exceptions:
The sequence coding for cytochrome P450 CYP73A1 is replaced by the sequence coding for cytochrome P450 CYP76F112 with the sequence

(SEQ ID NO 45)
caagtcacgacggttgtcatttcctccaccaccatggctaaagaagtcct
ccaggcaaacagccaagtcgtctccagccggacaatcaccgacgcaagcc
gcgcccacagacacagcgattttagcatggttatgttgcccgtatcccct
ctgtggcgaaaccttcggaaaataagcaactcacacttgctttcctccaa
ggctcttgatggcaacatggagctgagaaacaaaaaggtgcaagagctcc
taaatgatgtccacaaaagcgtccaggccggggaggcggtggagatcgcg
agcctttctttcagagctactctgaatctcttgtccaccacatttttctc
catggacatggcggatgacacaaattccgtcactctaaaagagctcaagg
aggctatgtcgcacatgatggaagagttggggaagcctaacttggccgat
tatttcccgtttctacaaaagattgacccccaaggcattaggcggcgcaa
cacggttactttccggaaactgatcaacttgtttgggcgtatcatcgacc
aaagattgaaagtgagagaagcgagtggttctttgaaagatgatgatatt
ttagacactcttatcaacatgatggtggtggatcaggagaagaaagagga
tcagcttgacaaaaccataattgaacattttttactggatttattttcag
cggggactgaaacgacttcaaccacgttggagtgggcaatggctgagcta
gtaaaagcgccagagattatgtcaaaagcccgagcagagctagatcaagt
tataggcaaaggaaaccaagtgaaggaatcggacgtatctcgactccctt
acttacaagccattgttaaagaaaccttccgcatgcaccctacagctcca
ttattgattcctcgcaaagccgacagtgacatcgaaatctccgactatat
catcccgaaggatgctcag
or
(SEQ ID NO 71)
aaacctcgtcccatcatcggaagcctcttggagctcggcgaccaacccca
caggtccttggccaggctttccgagtcttacggcccgtttatgcatttga
agctcggccaagtcacgacggttgtcatttcctccaccaccatggctaaa
gaagtcctccaggcaaacagccaagtcgtctccagccggacaatcaccga
cgcaagccgcgcccacagacacagcgattttagcatggttatgttgcccg
tatcccctctgtggcgaaaccttcggaaaataagcaactcacacttgctt
tcctccaaggctcttgatggcaacatggagctgagaaacaaaaaggtgca
agagctcctaaatgatgtccacaaaagcgtccaggccggggaggcggtgg
agatcgcgagcctttctttcagagctactctgaatctcttgtccaccaca
tttttctccatggacatggoggatgacacaaattccgtcactctaaaaga
gctcaaggaggctatgtcgcacatgatggaagagttggggaagcctaact
tggccgattatttcccgtttctacaaaagattgacccccaaggcattagg
cggcgcaacacggttactttccggaaactgatcaacttgtttgggcgtat
catcgaccaaagattgaaagtgagagaagcgagtggttctttgaaagatg
atgatattttagacactcttatcaacatgatggtggtggatcaggagaag
aaagaggatcagcttgacaaaaccataattgaacattttttactggattt
attttcagcggggactgaaacgacttcaaccacgttggagtgggcaatgg
ctgagctagtaaaagcgccagagattatgtcaaaagcccgagcagagcta
gatcaagttataggcaaaggaaaccaagtgaaggaatcggacgtatctcg
actcccttacttacaagccattgttaaagaaaccttccgcatgcacccta
cagctccattattgattcctcgcaaagccgacagtgacatcgaaatctcc
gactatatcatcccgaaggatgctcaggtgattgtcaatgtatgggccat
tggtagagactcaagcacatgggaaaatcccgacaagtttataccggaga
ggtttttggacatcgatatagatgtcggaggccgggattttaagctcatt
ccgttcggtgctggtcggagaatatgtcccggattcccattggcgatgcg
aatgttgcacttgatgttggggtctttgcttcactcgtttgattggaagt
tggaagatggggttagacctgatgctctaaacatggatgaaaagtttggc
ctcaccttgcaaatggctcagcctttgcgagctatccccgtgccgacaaa
gcat

The cinnamic acid substrate is replaced by demethylsuberosine. Bioconversion of the substrate generates marmesin as shown in FIG. 9 (CYP76F112).

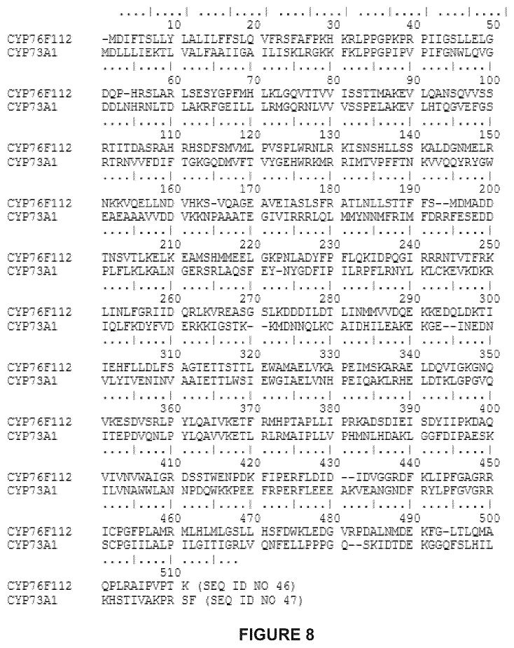

A comparison of the peptide sequences of cytochromes P450 CYP76F112 and P450 CYP73A1 by peptide sequence alignment was carried out using the Basic Local Alignment Search Tool (BLAST) and is shown in FIG. 8. The result shows a percent identity of 28.7%.

In this example, bioconversion is carried out according to the process described in Example 1 above, wherein the substrate used is demethylsuberosine,

This example clearly demonstrates that an example fusion protein successively comprising (i) at least one polypeptide for targeting and anchoring to the bacterial membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of a NADPH P450 reductase of P450 cytochrome of plant, enables the bioconversion of the substrate.

This example also clearly demonstrates that an example of a fusion protein according to the invention can be expressed on the surface of a cell, in particular a bacterial cell, and can advantageously be used in a substrate bioconversion process.

This example also clearly demonstrates that an example of a fusion protein according to the invention can be expressed on the surface of a cell, in particular a bacterial cell, and can advantageously enable bioconversion of substrates in the culture medium of said cell.

### Example 3: Process for Preparing a Fusion Protein, and Biotransformation Process Using Said Protein

In this example, the cytochrome P450 is cytochrome CYP76F112 (marmesin synthase, Ficus carica cytochrome P450 CYP76F112 mRNA, complete cds Sequence ID: MW348922.1, GenBank: MW348922.1), whose sequence is

(SEQ ID NO 71)
aaacctcgtcccatcatcggaagcctcttggagctcggcgaccaacccca
caggtccttggccaggctttccgagtcttacggcccgtttatgcatttga
agctcggccaagtcacgacggttgtcatttcctccaccaccatggctaaa
gaagtcctccaggcaaacagccaagtcgtctccagccggacaatcaccga
cgcaagccgcgcccacagacacagcgattttagcatggttatgttgcccg
tatcccctctgtggcgaaaccttcggaaaataagcaactcacacttgctt
tcctccaaggctcttgatggcaacatggagctgagaaacaaaaaggtgca
agagctcctaaatgatgtccacaaaagcgtccaggccggggaggcggtgg
agatcgcgagcctttctttcagagctactctgaatctcttgtccaccaca
tttttctccatggacatggcggatgacacaaattccgtcactctaaaaga
gctcaaggaggctatgtcgcacatgatggaagagttggggaagcctaact
tggccgattatttcccgtttctacaaaagattgacccccaaggcattagg
cggcgcaacacggttactttccggaaactgatcaacttgtttgggcgtat
catcgaccaaagattgaaagtgagagaagcgagtggttctttgaaagatg
atgatattttagacactcttatcaacatgatggtggtggatcaggagaag
aaagaggatcagcttgacaaaaccataattgaacattttttactggattt
attttcagcggggactgaaacgacttcaaccacgttggagtgggcaatgg
ctgagctagtaaaagcgccagagattatgtcaaaagcccgagcagagcta
gatcaagttataggcaaaggaaaccaagtgaaggaatcggacgtatctcg
actcccttacttacaagccattgttaaagaaaccttccgcatgcacccta
cagctccattattgattcctcgcaaagccgacagtgacatcgaaatctcc
gactatatcatcccgaaggatgctcaggtgattgtcaatgtatgggccat
tggtagagactcaagcacatgggaaaatcccgacaagtttataccggaga
ggtttttggacatcgatatagatgtcggaggccgggattttaagctcat
tccgttcggtgctggtcggagaatatgtcccggattcccattggcgatgc
gaatgttgcacttgatgttggggtctttgcttcactcgtttgattggaag
ttggaagatggggttagacctgatgctctaaacatggatgaaaagtttgg
cctcaccttgcaaatggctcagcctttgcgagctatccccgtgccgacaa
agcat (nucleic acid coding for the hydrophilic
domain of cytochrome P450 CYP76F112).

A comparison of the peptide sequences of cytochromes P450 CYP76F112 and P450 CYP73A1 by peptide sequence alignment was carried out using the Basic Local Alignment Search Tool (BLAST) and is shown in FIG. 8. The result shows a percent identity of 28.7%.

1) Construction of a Generic Expression Plasmid pAIDA-T7

The basis of the expression plasmid is based on a commercial plasmid pAIDA1 (https://www.addgene.org/79180/[11]), which is a low-copy plasmid.

### Replacement of the Selection Gene

The pAIDA1 plasmid was first modified by replacing the gene conferring chloramphenicol resistance with a gene conferring tetracycline resistance cloned from the pBR322 plasmid marketed by Fisher Scientific (https://www.fishersci.fr/shop/products/fermentas-pbr322-dna/10191220 [11]), as described in example 1 above. To this end, plasmid pAIDA1 was used as the template, which was amplified by polymerase chain reaction (PCR) using the enzyme PrimeSTAR Max polymerase marketed by Takara Bio Inc, according to the procedure Protocol 1: PrimeSTAR Max polymerase Protocol as described in (https://www.takarabio.com/documents/User%20Manual/R045A_e.v2102 Da.pdf [6]). Amplification was performed using primers LAEWXpr17-lacIQ (tggcgacaccatcgaatggtgc (SEQ ID NO: 21) and LAEWXpr02: Reverse: tttagcttccttagctcctg (SEQ ID NO: 22). This amplification enabled the plasmid to be copied in its entirety, with the exception of the chloramphenicol resistance gene. The same process was used to amplify the sequence coding for the tetracycline resistance gene. Amplification was performed this time from plasmid pBR322 using primers LAEWXpr03 (gctaaggaagctaaaatgaaatctaacaatgcgct (SEQ ID NO 41)) and LAEWXpr04 (tcgatggtgtcgccacgctgcccgagatgc (SEQ ID NO 42)). The two PCR products obtained, that is, the pAIDA1 plasmid without the chloramphenicol resistance gene and the sequence coding for the tetracycline resistance gene, were fused using the In-Fusion kit marketed by Takara Bio Inc, according to the Protocol 2: In-Fusion Protocol described in the document https://www.takarabio.com/documents/User%20Manual/In/In-Fusion%20Snap%20Assembly%20User%20Manual_071320.pdf [16]. The recombinant plasmid obtained was introduced into chemocompetent Escherichia coli TOP10 bacteria marketed by Life Technologies Corporation according to the TOP10 transformation protocol described in https://assets.thermofisher.com/TFS-Assets/LSG/manuals/oneshottop10_man.pdf [17]. The transformed bacteria were plated on an LB (lysogenic broth) culture medium (10 g peptone, 5 g yeast extract, 5 g NaCl) containing tetracycline. The insertion of the gene coding for tetracycline resistance was verified by PCR using primers LAEWXpr17 (SEQ ID NO 21) and LAEWXpr02 (SEQ ID NO 22).

The recombinant plasmid pAIDA1-TetR-lacI^{Q}, shown in FIG. 2, was used to construct the other plasmids. To simplify the nomenclature, the plasmid is also referred to as pAIDA1. This plasmid was amplified and purified from a positive colony using the Protocol 3 plasmid purification protocol described in the document https://www.mn-net.com/media/pdf/45/51/02/Instruction-NucleoSpin-Plasmid.pdf [18].

### a) Promoter and Terminator

The promoter and terminator of the AIDA cassette of plasmid pAIDA1 were replaced by two cloning cassettes featuring the promoter and terminator of T7 RNA Polymerase as described in example 1 above. To carry out this step, various cloning techniques were used
(i) pAIDA1 was amplified by PCR using primers LAEWXpr30 (tcatcatcatgcctaatgagtgagaattcc (SEQ ID NO 23)) and LAEWXpr31 (ttggtgcgcaaactattaactgg (SEQ ID NO 24)). This amplification step involves the DNA fragment contained between the pAIDA1 promoter and the pAIDA1 terminator. The amplification product therefore corresponded to the plasmid without promoter and terminator
(ii) In parallel, the T7 terminator was amplified by PCR from plasmid pET28b-2 using primers LAEWXpr32-1-fr (ctcattaggcatgatgatgaaaggaagggaagaaagcgaaagg (SEQ ID NO 25)) and LAEWXpr32-2-rv (agtactcctaggactagtggtaccagatccggctgctaacaaagc (SEQ ID NO 26)). Finally, the T7 promoter was amplified by PCR from plasmid pET28b-2 using primers LAEWXpr33-1-rv (gttaatagtttgcgcaccaaatcggtgatgtcggcgatatagg (SEQ ID NO 27)) and LAEWXpr33-2-fr (ggtaccactagtcctaggagtactatggctgctgcccatggtata (SEQ ID NO 28)) according to Protocol 1 as stated above.
(iii) The three fragments were fused using the In-Fusion kit according to Protocol 2 as stated above. This ligation product allow to generate the pAIDA1-T7 plasmid shown in FIG. 3.

### B) Inserting the AIDA1 Cassette

The AIDA sequence of the original pAIDA1 plasmid was amplified by PCR using primers LAEWXpr36-AIDA-fr (aactttaagaaggagatataccatgggcaataaggcctacagtatcatttgg (SEQ ID NO 29)) and LAEWXpr37-AIDA-rv (tttgttagcagccggatctgtcattatcagaagctgtattttatc (SEQ ID NO 30)) according to Protocol 1 as mentioned above.

In parallel, plasmid pAIDA1-T7 was digested with NcoI and KpnI restriction enzymes according to the Protocol 4 digestion protocol as described in http://assets.thermofisher.com/TFS-Assets/BID/Reference-Materials/fastdigest-restriction-enzymes-labaid.pdf [19]. The linearized pAIDA1-T7-NcoI-KpnI plasmid and the AIDA1 amplicon were fused by In-Fusion according to Protocol 2 above. The generic recombinant plasmid pAIDA1-T7 complete with AIDA1 was amplified by bacterial transformation. The obtained complete generic plasmid pAIDA1-T7 with AIDA1 is shown in FIG. 4.

### 2) Construction of a Recombinant Plasmid for Expression of a P450-ATR Fusion Protein

a) Plasmid pAIDA1-T7 was linearized and amplified by PCR using primers Vector 5 ATR1-FL-76F112 fwd (cgacaaagcatgagctcgaaaacctgtacttcc (SEQ ID NO 48)) and New Vector 5 rvs (agactctagtggtacccggaccctggaaca (SEQ ID NO 49)) according to Protocol 1 as stated above. The obtained linearized plasmid sequence corresponds to the sequence

(SEQ ID NO 50)
agactctagtggtacccggaccctggaacagcgcttccagatggtgatggtgatggtggtcgactgcaaatgcatttccgatt
gtggaaacaaccgccaataccagcagtgtattttttgcaaggacaaaaccatgtcctctggctaactctgaggccacaatcc
aggcctgtctggagtggctccaaatgatactgtaggccttattgcccatggtatatctccttcttaaagttaaacaaaattatttcta
gaggggaattgttatccgctcacaattcccctatagtgagtcgtattaatttcgcgggatcgagatctcgatcctctacgccgga
cgcatcgtggccggcatcaccggcgccacaggtgcggttgctggcgcctatatcgccgacatcaccgatttggtgcgcaaa
ctattaactggcgaactacttactctagcttcccggcaacaattaatagactggatggaggcggataaagttgcaggaccact
tctgcgctcggcccttccggctggctggtttattgctgataaatctggagccggtgagcgtgggtctcgcggtatcattgcagca
ctggggccagatggtaagccctcccgtatcgtagttatctacacgacggggagtcaggcaactatggatgaacgaaataga
cagatcgctgagataggtgcctcactgattaagcattggtaactgtcagaccaagtttactcatatatactttagattgatttaaa
acttcatttttaatttaaaaggatctaggtgaagatcctttttgataatctcatgaccaaaatcccttaacgtgagttttcgttccactg
agcgtcagaccccttaataagatgatcttcttgagatcgttttggtctgcgcgtaatctcttgctctgaaaacgaaaaaaccgcct
tgcagggcggtttttcgaaggttctctgagctaccaactctttgaaccgaggtaactggcttggaggagcgcagtcaccaaaa
cttgtcctttcagtttagccttaaccggcgcatgacttcaagactaactcctctaaatcaattaccagtggctgctgccagtggtg
cttttgcatgtctttccgggttggactcaagacgatagttaccggataaggcgcagcggtcggactgaacggggggttcgtgc
atacagtccagcttggagcgaactgcctacccggaactgagtgtcaggcgtggaatgagacaaacgcggccataacagc
ggaatgacaccggtaaaccgaaaggcaggaacaggagagcgcacgagggagccgccagggggaaacgcctggtat
ctttatagtcctgtcgggtttcgccaccactgatttgagcgtcagatttcgtgatgcttgtcaggggggcggagcctatggaaaa
acggctttgccgcggccctctcacttccctgttaagtatcttcctggcatcttccaggaaatctccgccccgttcgtaagccatttc
cgctcgccgcagtcgaacgaccgagcgtagcgagtcagtgagcgaggaagcggaatatatcctgtatcacatattctgctg
acgcaccggtgcagccttttttctcctgccacatgaagcacttcactgacaccctcatcagtgccaacatagtaagccagtata
cactccgctagcgctgaggtctgcctcgtgaagaaggtgttgctgactcataccaggcctgaatcgccccatcatccagcca
gaaagtgagggagccacggttgatgagagctttgttgtaggtggaccagttggtgattttgaacttttgctttgccacggaacgg
tctgcgttgtcgggaagatgcgtgatctgatccttcaactcagcaaaagttcgatttattcaacaaagccacgttgtgtctcaaa
atctctgatgttacattgcacaagataaaaatatatcatcatgaacaataaaactgtctgcttacataaacagtaatacaaggg
gtgttatgagccatattcaacgggaaacgtcttgctcgagtatccgctcatgagattatcaaaaaggatcttcacctagatccttt
tgtaagttctcatgtttgacagcttatcatcgataagctttaatgcggtagtttatcacagttaaattgctaacgcagtcaggcacc
gtgtatgaaatctaacaatgcgctcatcgtcatcctcggcaccgtcaccctggatgctgtaggcataggcttggttatgccggta
ctgccgggcctcttgcgggatatcgtccattccgacagcatcgccagtcactatggcgtgctgctagcgctatatgcgttgatgc
aatttctatgcgcacccgttctcggagcactgtccgaccgctttggccgccgcccagtcctgctcgcttcgctacttggagccac
tatcgactacgcgatcatggcgaccacacccgtcctgtggatcctctacgccggacgcatcgtggccggcatcaccggcgc
cacaggtgcggttgctggcgcctatatcgccgacatcaccgatggggaagatcgggctcgccacttcgggctcatgagcgc
ttgtttcggcgtgggtatggtggcaggccccgtggccgggggactgttgggcgccatctccttgcatgcaccattccttgcggc
ggcggtgctcaacggcctcaacctactactgggctgcttcctaatgcaggagtcgcataagggagagcgtcgaccgatgcc
cttgagagccttcaacccagtcagctccttccggtgggcgcggggcatgactatcgtcgccgcacttatgactgtcttctttatca
tgcaactcgtaggacaggtgccggcagcgctctgggtcattttcggcgaggaccgctttcgctggagcgcgacgatgatcgg
cctgtcgcttgcggtattcggaatcttgcacgccctcgctcaagccttcgtcactggtcccgccaccaaacgtttcggcgagaa
gcaggccattatcgccggcatggcggccgacgcgctgggctacgtcttgctggcgttcgcgacgcgaggctggatggcctt
ccccattatgattcttctcgcttccggcggcatcgggatgcccgcgttgcaggccatgctgtccaggcaggtagatgacgacc
atcagggacagcttcaaggatcgctcgcggctcttaccagcctaacttcgatcattggaccgctgatcgtcacggcgatttatg
ccgcctcggcgagcacatggaacgggttggcatggattgtaggcgccgccctataccttgtctgcctccccgcgttgcgtcgc
ggtgcatggagccgggccacctcgacctgaatggaagccggcggcacctcgctaacggattcaccactccaagaattgg
agccaatcaattcttgcggagaactgtgaatgcgcaaaccaacccttggcagaacatatccatcgcgtccgccatctccagc
agccgcacgcggcgcatctcgggcagcgtggcgacaccatcgaatggtgcaaaacctttcgcggtatggcatgatagcgc
ccggaagagagtcaattcagggtggtgaatgtgaaaccagtaacgttatacgatgtcgcagagtatgccggtgtctcttatca
gaccgtttcccgcgtggtgaaccaggccagccacgtttctgcgaaaacgcgggaaaaagtggaagcggcgatggcgga
gctgaattacattcccaaccgcgtggcacaacaactgggggcaaacagtcgttgctgattggcgttgccacctccagtctg
gccctgcacgcgccgtcgcaaattgtcgcggcgattaaatctcgcgccgatcaactgggtgccagcgtggtggtgtcgatgg
tagaacgaagcggcgtcgaagcctgtaaagcggcggtgcacaatcttctcgcgcaacgcgtcagtgggctgatcattaact
atccgctggatgaccaggatgccattgctgtggaagctgcctgcactaatgttccggcgttatttcttgatgtctctgaccagaca
cccatcaacagtattattttctcccatgaagacggtacgcgactgggcgtggagcatctggtcgcattgggtcaccagcaaat
cgcgctgttagcgggcccattaagttctgtctcggcgcgtctgcgtctggctggctggcataaatatctcactcgcaatcaaatt
cagccgatagcggaacgggaaggcgactggagtgccatgtccggttttcaacaaaccatgcaaatgctgaatgagggcat
cgttcccactgcgatgctggttgccaacgatcagatggcgctgggcgcaatgcgcgccattaccgagtccgggctgcgcgtt
ggtgcggatatctcggtagtgggatacgacgataccgaagacagctcatgttatatcccgccgttaaccaccatcaaacag
gattttcgcctgctggggcaaaccagcgtggaccgcttgctgcaactctctcagggccaggcggtgaagggcaatcagctgt
tgcccgtctcactggtgaaaagaaaaaccaccctggcgcccaatacgcaaaccgcctctccccgcgcgttggccgattcat
taatgcagctggcacgacaggtttcccgactggaaagcgggcaagtgagtggataaccgtattaccgcctttgagtgagctg
ataccgggaattctcactcattaggcatgatgatgaaaggaagggaagaaagcgaaaggagcgggcgctagggcgctg
gcaagtgtagcggtcacgctgcgcgtaaccaccacacccgccgcgcttaatgcgccgctacagggcgcgtcccattcgcc
aatccggatatagttcctcctttcagcaaaaaacccctcaagacccgtttagaggccccaaggggttatgctagttattgctca
gcggtggcagcagccaactcagcttcctttcgggctttgttagcagccggatctgtcattatcagaagctgtattttatccccagt
gctccggagatggcattgctcccgtgacctcctgcctgatatgcgactccgccattcactgacaagttttgagtaatcacccctt
caatacctgtctttatctctccctgatttcggctacctgacaacaactggctgtcatcactcattttaacaccaaattcatgagtgtt
atggatccagtttgcctctatatacggacggaacctccgcccggtatccttatccagggtgcttttcaccttccaggatgcacga
atacctgcttttgtctgaatattatttttccctgctccctgcaccaccgttccgttatcctcctgatgtgtatccggtgtaacccccatcc
agacagcctgcaaatgaggctgtaaccagaattcacctgttattccttcaggtgatgtccatgtgtgcacattcaggttatatccc
ccacctgcagaagcggttaaaccattcagattatatttttcttcttccagtccgtcacctttcactgatgcattaaaccagttatattg
catccaagtttcagcaaagagccctgttgcattttccccattctgataccacgtaccgtataccccgacagaataaccatccag
tgtgtttctggcagctttgttgctcgtgtaatttatcgttttaccttttgcattcgcgtatcctcccataatccctaaggtaaaatcaccc
agttgttcagcatggaatttataaatatcccccccgagctgattgataaactgattggttgttgttttattttgcccgtcattcagctta
ccagagcttattcctccagtgatcttcatccacacggatgcagactcaggctgtgtattatcactcatggccctgaattgcttacg
ctcattcaaatccatgaggaacagtgagttagccagtgccatattggtagcataacttccgttctccggtctgtattgccgggtat
cagatgtgggaagatgactggttaaataccatcccttattatctgtcccactctcgtttcctttctgcagtgtgtaatcataagctcc
ggcaactacgcggttcttcagagagaattctgcatcagaatttccctctacagaaataatattaataccatctctcgtctgacca
ccactgccatcttcattgacataaacgatgtcactttgaccagaggtattacctttcaccaccagacggtccgtaagtgaattat
ctccttcaagcacaccaccaagagaaataacacttcccggtgtcccagtataatttgacacggtaagagtattacctatagcg
gccgcactttcttttgtaggattaagaatgatatttttgttattaacaagactaccattcactgtagcagagagcagctttgctgtac
caaaacttaagtcagcatcattcgtaatattcccgtttataatgctgttattaatgacaatgcttccattgttattcactctagacaga
tcttcttcgctaatcagtttctgttcaccctggaagtacaggttttcgagctcatgctttgtcg.

b) The sequence coding for the extra-membrane portion of Arabidopsis thaliana NADPH P450 reductase 1 (ATR1) as described in Urban et al, (1997) J Biol Chem 272(31):19176-86 [20]) was amplified by PCR from a plasmid (pCR8_ATR1) using primers ATR1 fwd 16123 Forward (tccgggtaccactagagtctctatcttcttcggt (SEQ ID NO 51)) and ATR1 rvs redo (ccgggagctcccagacatctctgaggtatcttc (SEQ ID NO 52)) according to Protocol 1 as mentioned above).

c) The linker (rich in GC pairs) was amplified from a synthetic sequence using the PrimeSTAR GXL DNA Polymerase enzyme marketed by Takara Bio Inc. using primers FLfwd redo (agatgtctgggagctcccgggcggttctggt (SEQ ID NO 53)) and FLrvs 76F112 (ggacgaggtttctcgagcggagaaccgccgct (SEQ ID NO 54) according to two-step Protocol 5 described in PrimeSTAR GXL DNA Polymerase Manual, https://www.takara.co.kr/file/manual/pdf/R050A_e.v1906 Da.pdf [21]).

d) The DNA sequence coding for the hydrophilic extra-membrane portion (SEQ ID 71) of P450, CYP76F112 (Villard et al, (2021) New Phytologist 231 (5): 1923-1939 [22]) was amplified by PCR from a pcr8-CYP76F112 plasmid described in Villard et al, (2021) New Phytologist 231 (5):1923-1939 [22]) using primers 76F112 fwd (tccgctcgagaaacctcgtcccatcatcgga (SEQ ID NO 55)) and 76F112 rvs (tttcgagctcatgctttgtcggcacgggggga (SEQ ID NO 56)) according to Protocol 1 mentioned above. The coding sequence

e) The 3 DNA fragments from sequences SEQ ID NO 71, SEQ ID NO 15, SEQ ID NO 17 were fused by PCR fusion. PCR fusion consists of using the 3 DNA fragments at equimolar concentration as a template for a PCR reaction, and hybridizing the 3 sequences into a single one via their homologous parts, enabling them to be amplified by PCR reaction, according to the above-mentioned Protocol 4, a sequence corresponding to

(SEQ ID NO 57)
tccgggtaccactagagtctctatcttcttcggtacgcagactggaacagctgagggatttgctaaggcattatccgaagaa
atcaaagcgagatatgaaaaagcagcagtcaaagtcattgacttggatgactatgctgccgatgatgaccagtatgaag
agaaattgaagaaggaaactttggcatttttctgtgttgctacttatggagatggagagcctactgacaatgctgccagatttt
acaaatggtttacggaggaaaatgaacgggatataaagcttcaacaactagcatatggtgtgtttgctcttggtaatcgcca
atatgaacattttaataagatcgggatagttcttgatgaagagttatgtaagaaaggtgcaaagcgtcttattgaagtcggtct
aggagatgatgatcagagcattgaggatgattttaatgcctggaaagaatcactatggtctgagctagacaagctcctcaa
agacgaggatgataaaagtgtggcaactccttatacagctgttattcctgaataccgggtggtgactcatgatcctcggttta
caactcaaaaatcaatggaatcaaatgtggccaatggaaatactactattgacattcatcatccctgcagagttgatgttgct
gtgcagaaggagcttcacacacatgaatctgatcggtcttgcattcatctcgagttcgacatatccaggacgggtattacata
tgaaacaggtgaccatgtaggtgtatatgctgaaaatcatgttgaaatagttgaagaagctggaaaattgcttggccactctt
tagatttagtattttccatacatgctgacaaggaagatggctccccattggaaagcgcagtgccgcctcctttccctggtccat
gcacacttgggactggtttggcaagatacgcagaccttttgaaccctcctcgaaagtctgcgttagttgccttggcggcctat
gccactgaaccaagtgaagccgagaaacttaagcacctgacatcacctgatggaaaggatgagtactcacaatggatt
gttgcaagtcagagaagtcttttagaggtgatggctgcttttccatctgcaaaacccccactaggtgtattttttgctgcaatag
ctcctcgtctacaacctcgttactactccatctcatcctcgccaagattggcgccaagtagagttcatgttacatccgcactagt
atatggtccaactcctactggtagaatccacaagggtgtgtgttctacgtggatgaagaatgcagttcctgcggagaaaagt
catgaatgtagtggagccccaatctttattcgagcatctaatttcaagttaccatccaacccttcaactccaatcgttatggtgg
gacctgggactgggctggcaccttttagaggttttctgcaggaaaggatggcactaaaagaagatggagaagaactagg
ttcatctttgctcttctttgggtgtagaaatcgacagatggactttatatacgaggatgagctcaataattttgttgatcaaggcgt
aatatctgagctcatcatggcattctcccgtgaaggagctcagaaggagtatgttcaacataagatgatggagaaggcag
cacaagtttgggatctaataaaggaagaaggatatctctatgtatgcggtgatgctaagggcatggcgagggacgtccac
cgaactctacacaccattgttcaggagcaggaaggtgtgagttcgtcagaggcagaggctatagttaagaaacttcaaac
cgaaggaagatacctcagagatgtctgggagctcccgggcggttctggtggcggtagcggcggtggcggttctggcggt
ggcggtagcggcggtggcggttctggcggtggcggtagcggcggtggcggttctggcggtggcggtagcggcggtggc
ggttctggtggcggtagcggcggttctccgctcgagaaacctcgtcccatcatcggaagcctcttggagctcggcgaccaa
ccccacaggtccttggccaggctttccgagtcttacggcccgtttatgcatttgaagctcggccaagtcacgacggttgtcatt
tcctccaccaccatggctaaagaagtcctccaggcaaacagccaagtcgtctccagccggacaatcaccgacgcaagc
cgcgcccacagacacagcgattttagcatggttatgttgcccgtatcccctctgtggcgaaaccttcggaaaataagcaact
cacacttgctttcctccaaggctcttgatggcaacatggagctgagaaacaaaaaggtgcaagagctcctaaatgatgtcc
acaaaagcgtccaggccggggaggcggtggagatcgcgagcctttctttcagagctactctgaatctcttgtccaccacat
ttttctccatggacatggcggatgacacaaattccgtcactctaaaagagctcaaggaggctatgtcgcacatgatggaag
agttggggaagcctaacttggccgattatttcccgtttctacaaaagattgacccccaaggcattaggcggcgcaacacgg
ttactttccggaaactgatcaacttgtttgggcgtatcatcgaccaaagattgaaagtgagagaagcgagtggttctttgaaa
gatgatgatattttagacactcttatcaacatgatggtggtggatcaggagaagaaagaggatcagcttgacaaaaccata
attgaacattttttactggatttattttcagcggggactgaaacgacttcaaccacgttggagtgggcaatggctgagctagta
aaagcgccagagattatgtcaaaagcccgagcagagctagatcaagttataggcaaaggaaaccaagtgaaggaatc
ggacgtatctcgactcccttacttacaagccattgttaaagaaaccttccgcatgcaccctacagctccattattgattcctcg
caaagccgacagtgacatcgaaatctccgactatatcatcccgaaggatgctcaggtgattgtcaatgtatgggccattggt
agagactcaagcacatgggaaaatcccgacaagtttataccggagaggtttttggacatcgatatagatgtcggaggccg
ggattttaagctcattccgttcggtgctggtcggagaatatgtcccggattcccattggcgatgcgaatgttgcacttgatgttg
gggtctttgcttcactcgtttgattggaagttggaagatggggttagacctgatgctctaaacatggatgaaaagtttggcctc
accttgcaaatggctcagcctttgcgagctatccccgtgccgacaaagcatgagctcgaaa.

f) The ATR1-FL-76F112 DNA fragment (SEQ ID NO 57) obtained by PCR fusion and the linearized plasmid pAIDA1-T7 (SEQ ID NO 50) were then fused by Infusion according to Protocol 2. The ligation product was introduced into E. coli HST08 (Takara) bacteria. The transformed bacteria were plated on solid LB (lysogenic broth) medium (10 g peptone, 5 g yeast extract, 5 g NaCl, 16 g Agar) containing tetracycline (50 μg/mL) at 37° C. An isolated colony was then used to amplify the plasmid pAIDA1-T7-CYP76F112-ATR1 by culture in liquid LB medium (without Agar) containing tetracycline (50 μg/mL) at 37° C. The presence of plasmid constitutive elements was verified by PCR using primers

| | |
|---|---|
| 27F | (GCTAGAGTAAGTAGTTCGCCAGT (SEQ ID NO 72)), |
| 51 F | (CCTGAATACCGGGTGGTGAC (SEQ ID NO 58)), |
| 52R | (GCATATACACCTACATGGTCAC (SEQ ID NO 59)), |
| 53F | (GTGAAGGAGCTCAGAAGGAGTA (SEQ ID NO 60)), |
| 54R | (TAGAGTTCGGTGGACGTCCCT (SEQ ID NO 61)), |
| 62F | (TTTGGGCGTATCATCGACCAA (SEQ ID NO 62)), |
| 63R | (ATGGTTTTGTCAAGCTGATCCTC (SEQ ID NO 63)), |
| 57F | (TTCTCTTGGTGGTGTGCTTGA (SEQ ID NO 64), |
| 58R | (catctctcgtctgaccacca (SEQ ID NO 65)), |
| 59F | (ATAACGGAACGGTGGTGCAGG (SEQ ID NO 66)), |
| 60R | (AACCTCCGCCCGGTATCCTT (SEQ ID NO 67)) |

and

| | |
|---|---|
| 61 R | (ACGATACCGAAGACAGCTCATG (SEQ ID NO 68)). |

These steps led to the construction
of a plasmid pAIDA1-T7-CYP76F112- ATR1

### 3) Fusion Protein

The resulting fusion protein contains from the N-terminus to the C-terminus:
a 6-Histidine tag, enabling protein immunodetection with anti-Histidine antibodies
a cleavage sequence to remove the tag,
a c-MYC sequence, enabling protein immunodetection with anti-c-MYC antibodies
an AIDA1 binding peptide
an outer membrane anchoring and addressing polypeptide: the AIDA1 beta Barel anchoring sequence, that is, SEQ ID NO 1
a polypeptide sequence of cytochrome P450 CYP76F112 (marmesin synthase from Ficus carica, NCBI ID: GenBank Sequence ID: MW348922.1) wherein the membrane anchor has been removed from the coding sequence, namely SEQ ID NO 70,
a 51-amino-acid binding peptide PGGSGGGSGGGGSGGGGSGGGGSGGGGSGGGGSGGGGSGGG GSGGGSGGSP (SEQ ID NO 6), and
a polypeptide sequence of Arabidopsis NADPH P450 reductase (Arabidopsis thaliana P450 reductase 1 (ATR1), mRNA-NCBI Reference Sequence: NM_118585.4) wherein the membrane anchor has been removed, namely the sequence SEQ ID NO 7.

The coding nucleic sequences corresponding to the different elements of the fusion protein described above were used and assembled successively from 5′ to 3′. In particular, it involved:
the sequence SEQ ID NO 44 coding for an AIDA1 linker,
the sequence SEQ ID NO 10 coding for a polypeptide for targeting and anchoring to the external membrane: the beta barrel anchoring sequence
the sequence SEQ ID NO 71 coding for the cytochrome P450 CYP76F112 marmesin synthase from Ficus carica, NCBI ID: Sequence ID: MW348922.1)
the sequence SEQ ID NO 15 coding for a 51-amino-acid binding peptide, and
the sequence SEQ ID NO 17 coding for Arabidopsis NADPH P450 reductase (Arabidopsis thaliana P450 reductase 1 (ATR1), mRNA-Sequence ID: NM_118585.4)
   4) Expression of Recombinant Proteins in E. coli Bacteria

### a) the Expression System Used

The pAIDA1 plasmid and recombinant plasmids containing the genes coding for the fusion protein are introduced into E. coli BL21 (DE3) plysE” bacteria (Novagen's® pET Systems). BL21 (DE3) pLysE bacteria are adapted for the production of proteins under the control of the T7 promoter. BL21 (DE3) pLysE bacteria carry the lambda DE3 lysogen and contain the pLysE plasmid, which constitutively expresses the T7 lysozyme. The T7 lysozyme reduces basal expression of target genes by inhibiting T7 RNA polymerase. The BL21 (DE3) pLysE strain therefore provides tighter control of T7 RNA polymerase.

### B) Preparation of an Example Fusion Protein

The fusion protein is expressed from the plasmid described in point 3 above. The plasmid is introduced into BL21 plysE bacteria according to the protocol recommended by the supplier (https://tools.thermofisher.com/content/sfs/manuals/oneshotbl21_man.pdf [23]) The presence of plasmid components is verified by PCR using primers 27F (GCTAGAGTAAGTAGTTCGCCAGT (SEQ ID NO 72), 51 F (CCTGAATACCGGGTGGTGAC (SEQ ID NO 58)), 52R (gcatatacacctacatggtcac (SEQ ID NO 59)), 53F (GTGAAGGAGCTCAGAAGGAGTA (SEQ ID NO 60)), 54R (TAGAGTTCGGTGGACGTCCCT (SEQ ID NO 61)), 62F (TTTGGGCGTATCATCGACCAA (SEQ ID NO 62)), 63R (atggttttgtcaagctgatcctc (SEQ ID NO 63)), 57F (TTCTCTTGGTGGTGTGCTTGA (SEQ ID NO 64), 58R (CATCTCTCGTCTGACCACCA (SEQ ID NO 65)), 59F (ATAACGGAACGGTGGTGCAGG (SEQ ID NO 66)), 60R (aacctccgcccggtatcctt (SEQ ID NO 67)) and 61 R (ACGATACCGAAGACAGCTCATG (SEQ ID NO 68)) on the plasmid extract obtained from a BL21 plysE culture transformed with plasmid pAIDA1-T7-CYP76F112-ATR1.

A glycerol-coated stock of BL21 plysE bacteria containing the pAIDA1-T7-CYP76F112-ATR1 plasmid is then used to inoculate an overnight preculture according to the supplier's recommendations (in-vitrogen), that is, for 12-14 hours, in LB medium (10 g peptone, 5 g yeast extract, 5 g NaCl) containing tetracycline (50 μg/mL) and chloramphenicol (20 μg/mL) at 37° C. This preculture is used to inoculate 50 mL of LB (lysogenic broth) medium (10 g peptone, 5 g yeast extract, 5 g NaCl) containing tetracycline (50 μg/mL) and chloramphenicol (20 μg/mL) at a D0 _{550 nm}=0.05 in a sterile 250 mL Erlenmeyer flask. After approximately 2 hours of culture at 37° C. with 180 rpm agitation, the culture is stopped when its optical density reaches a D0 _{550 nm} of 0.4. The cultures are then cooled on ice for 10 minutes. The volume of culture medium is measured and antibiotics are re-added to a final concentration of 50 μg/mL Tetracycline and 20 μg/mL Chloramphenicol. Fusion protein production is induced by adding isopropyl β-D-1-thiogalactopyranoside (IPTG) at a concentration of 20 μM final for 24 h at 7° C., under 180 rpm agitation. After 24 h, optical density is adjusted to 0.4 by dilution in LB medium (4° C.). Bacteria are harvested by two successive low-speed centrifugations (4° C., 20 min, 1000×g and 4° C., 10 min, 4000×g).

The bacteria are then suspended in buffer (88 mM with additives of KCl 1 mM, MgSO₄ 4 mM, 5% v/v glycerol, at 4° C. (products ordered from Sigma Aldrich)).

FIG. 1 is a schematic representation of the resulting fusion protein, said fusion protein being anchored in the bacterial membrane.

### 5) Bioconversion

A study of the bioconversion I of demethyl suberosin is carried out. FIG. 9 (CYP76F112) shows the corresponding bioconversion reaction. For this purpose, demethyl suberosin (100 μM) as substrate and nicotinamide adenine dinucleotide phosphate (NADPH) (400 μM) are added to the medium comprising the resuspended E. coli bacteria obtained in point 4 above. The bioconversion process is carried out at 20° C. for 1 h, with stirring at 180 rpm. The reaction is stopped by extraction with 1 volume of Ethyl Acetate. The media are vortexed for 5 min followed by centrifugation at 4400×g to separate the organic and aqueous phases. The upper organic phase is recovered and evaporated by Vivaspin. The powder obtained is suspended in 100 μL of methanol. The extract thus obtained is analyzed by ultra-high-performance liquid chromatography coupled to a mass spectrum (UHPLC/MS/MS).

This example therefore clearly demonstrates that an example fusion protein successively comprising (i) at least one polypeptide for targeting and anchoring to the bacterial membrane, advantageously to the external membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of a NADPH P450 reductase of cytochrome P450 of plant, enables the bioconversion of the substrate.

This example also clearly demonstrates that an example of a fusion protein according to the invention can be expressed on the surface of a cell, in particular a bacterial cell, and can advantageously be used in a substrate bioconversion process.

This example also clearly demonstrates that an example of a fusion protein according to the invention can be expressed on the surface of a cell, in particular a bacterial cell, and can advantageously enable bioconversion of substrates in the culture medium of said cell.

### BIBLIOGRAPHIC REFERENCES

1. Guengerich and Macdonald, 1990, FASEB J. 4, pp 2453-2459
2. X U Jun et al. “The cytochrome P450 superfamily: Key players in plant development and defense” Journal of Integrative Agriculture 2015, 14(9): 1673-1686
3. Kenneth Jensen et al., Plant NADPH-cytochrome P450 oxidoreductases, Phytochemistry Volume 71, 2-3, February 2010, Pages 132-141
4. https://blog.addgene.org/plasmid-101-origin-of-replication
5. https://www.qiagen.com/cn/resources/faq?id=1f42840e-fbd7-4734-b0cd-e17372a9e5a4&lang=en
6. https://www.takarabio.eom/documents/User%20Manual/R045A_e.v2102 Da.pdf
7. WO 83/004261
8. Naso et al 2017, “Adeno-Associated Virus (AAV) as a Vector for Gene Therapy”, BioDrugs. 2017 August; 31 (4):317-334. doi: 10.1007/s40259-017-0234-5
9. Zincarelli 2008 “Analysis of AAV Serotypes 1-9 Mediated Gene Expression and Tropism in Mice After Systemic Injection” Mol Ther. 2008 June; 16(6):1073-80. doi: 10.1038/mt.2008.76. Epub 2008 Apr. 15
10. https://www.addgene.org/79180/
11. https://www.fishersci.fr/shop/products/fermentas-pbr322-dna/10191220
12. https://www.takarabio.com/products/cloning/in-fusion-cloning
13 https://www.fishersci.fr/shop/products/fermentas-pbr322-dna/10191220
14. Jarmander, J., Gustavsson, M., Do, T H. et al. A dual tag system for facilitated detection of surface expressed proteins in Escherichia coli. Microb Cell Fact 11, 118 (2012). https://doi.org/10.1186/1475-2859-11-118
15. Rosano G L and Ceccarelli E A (2014) Recombinant protein expression in Escherichia coli: advances and challenges. Front. Microbiol. 5:172. doi: 10.3389/fmicb.2014.00172
16. https://www.takarabio.com/documents/User%20Manual/In/In-Fusion%20Snap%20Assembly%20User%20Manual_071320.pdf
17. https://assets.thermofisher.com/TFS-Assets/LSG/manuals/oneshottop10_man.pdf
18. https://www.mn-net.com/media/pdf/45/51/02/Instruction-NucleoSpin-Plasmid.pdf
19. http://assets.thermofisher.com/TFS-Assets/BID/Reference-Materials/fastdigest-restriction-enzymes-labaid.pdf
20. Urban et al, (1997) J Biol Chem 272(31):19176-86
21. https://www.takara.co.kr/file/manual/pdf/R050A_e.v1906 Da.pdf.
22. Villard, C., Munakata, R., Kitajima, S., Van Velzen, R., Schranz, M. E., Larbat, R., & Hehn, A. (2021). A new P450 involved in the furanocoumarin pathway underlies a recent case of convergent evolution. New Phytologist, 231(5), 1923-1939.
23. https://tools.thermofisher.com/content/sfs/manuals/oneshotbl21_man.pdf

1. A fusion protein successively comprising (i) at least one polypeptide for targeting and anchoring to the bacterial membrane, (ii) at least one polypeptide comprising the hydrophilic domain of a plant P450 cytochrome, (iii) at least one binding polypeptide comprising at least 47 amino acids, preferably comprising 51 amino acids and (iv) at least one polypeptide comprising the hydrophilic domain of NADPH P450 reductase of cytochrome P450 reductase of plant.

2. The fusion protein according to claim 1, wherein the quaternary structure of said at least one polypeptide for targeting and anchoring to the bacterial membrane forms a beta barrel.

3. The fusion protein according to claim 1, wherein the targeting and anchoring polypeptide is the polypeptide of sequence
(SEQ ID NO 1)
MNKAYSIIWSHSRQAWIVASELARGHGFVLAKNTLLVLAVVSTIGNAFAV
DHHHHHHLEALFQGPGTQKQRTELENLYFQGEQKLISEEDLSRVNNNGSI
VINNSIINGNITNDADLSFGTAKLLSATVNGSLVNNKNIILNPTKESAAA
IGNTLTVSNYTGTPGSVISLGGVLEGDNSLTDRLVVKGNTSGQSDIVYVN
EDGSGGQTRDGINIISVEGNSDAEFSLKNRVVAGAYDYTLQKGNESGTDN
KGWYLTSHLPTSDTRQYRPENGSYATNMALANSLFLMDLNERKQFRAMSD
NTQPESASVWMKITGGISSGKLNDGQNKTTTNQFINQLGGDIYKFHAEQL
GDFTLGIMGGYANAKGKTINYTSNKAARNTLDGYSVGVYGTWYQNGENAT
GLFAETWMQYNWFNASVKGDGLEEEKYNLNGLTASAGGGYNLNVHTWTSP
EGITGEFWLQPHLQAVWMGVTPDTHQEDNGTVVQGAGKNNIQTKAGIRAS
WKVKSTLDKDTGRRFRPYIEANWIHNTHEFGVKMSDDSQLLSGSRNQGEI
KTGIEGVITQNLSVNGGVAYQAGGHGSNAISGALGIKYSF.

4. The fusion protein according to claim 1, wherein said polypeptide of the hydrophilic domain of a plant P450 cytochrome is a polypeptide having a sequence identity of at least 28% identity with the polypeptide of sequence
(SEQ ID NO 3)
IPVPIFGNWLQVGDDLNHRNLTDLAKRFGEILLLRMGQRNLVVVSSPELA
KEVLHTQGVEFGSRTRNVVFDIFTGKGQDMVFTVYGEHWRKMRRIMTVPF
FTNKVVQQYRYGWEAEAAAVVDDVKKNPAAATEGIVIRRRLQLMMYNNMF
RIMFDRRFESEDDPLFLKLKALNGERSRLAQSFEYNYGDFIPILRPFLRN
YLKLCKEVKDKRIQLFKDYFVDERKKIGSTKKMDNNQLKCAIDHILEAKE
KGEINEDNVLYIVENINVAAIETTLWSIEWGIAELVNHPEIQAKLRHELD
TKLGPGVQITEPDVQNLPYLQAVVKETLRLRMAIPLLVPHMNLHDAKLGG
FDIPAESKILVNAWWLANNPDQWKKPEEFRPERFLEEEAKVEANGNDFRY
LPFGVGRRSCPGIILALPILGITIGRLVQNFELLPPPGQSKIDTDEKGGQ
FSLHILKHSTIVAKPRSF.

5. The fusion protein according to claim 1, wherein said polypeptide of the hydrophilic domain of a plant P450 cytochrome is a polypeptide selected from the group comprising the polypeptide of sequence
(SEQ ID NO 3)
IPVPIFGNWLQVGDDLNHRNLTDLAKRFGEILLLRMGQRNLVVVSSPELA
KEVLHTQGVEFGSRTRNVVFDIFTGKGQDMVFTVYGEHWRKMRRIMTVPF
FTNKVVQQYRYGWEAEAAAVVDDVKKNPAAATEGIVIRRRLQLMMYNNMF
RIMFDRRFESEDDPLFLKLKALNGERSRLAQSFEYNYGDFIPILRPFLRN
YLKLCKEVKDKRIQLFKDYFVDERKKIGSTKKMDNNQLKCAIDHILEAKE
KGEINEDNVLYIVENINVAAIETTLWSIEWGIAELVNHPEIQAKLRHELD
TKLGPGVQITEPDVQNLPYLQAVVKETLRLRMAIPLLVPHMNLHDAKLGG
FDIPAESKILVNAWWLANNPDQWKKPEEFRPERFLEEEAKVEANGNDFRY
LPFGVGRRSCPGIILALPILGITIGRLVQNFELLPPPGQSKIDTDEKGGQ
FSLHILKHSTIVAKPRSF
and
(SEQ ID NO 70)
KPRPIIGSLLELGDQPHRSLARLSESYGPFMHLKLGQVTTVVISSTTMAK
EVLQANSQVVSSRTITDASRAHRHSDFSMVMLPVSPLWRNLRKISNSHLL
SSKALDGNMELRNKKVQELLNDVHKSVQAGEAVEIASLSFRATLNLLSTT
FFSMDMADDTNSVTLKELKEAMSHMMEELGKPNLADYFPFLQKIDPQGIR
RRNTVTFRKLINLFGRIIDQRLKVREASGSLKDDDILDTLINMMVVDQEK
KEDQLDKTIIEHFLLDLFSAGTETTSTTLEWAMAELVKAPEIMSKARAEL
DQVIGKGNQVKESDVSRLPYLQAIVKETFRMHPTAPLLIPRKADSDIEIS
DYIIPKDAQVIVNVWAIGRDSSTWENPDKFIPERFLDIDIDVGGRDFKLI
PFGAGRRICPGFPLAMRMLHLMLGSLLHSFDWKLEDGVRPDALNMDEKFG
LTLQMAQPLRAIPVPTKH.

6. The fusion protein according to claim 1, wherein said binding polypeptide is a polypeptide of sequence
(SEQ ID NO 6)
PGGSGGGSGGGGSGGGGSGGGGSGGGGSGGGGSGGGGSGGGGSGGGSGGS
P.

7. The fusion protein according to claim 1, wherein said polypeptide of a hydrophilic domain of a NADPH P450 reductase is a polypeptide having a sequence identity of at least 90% with the polypeptide of sequence
(SEQ ID NO 7)
TRVSIFFGTQTGTAEGFAKALSEEIKARYEKAAVKVIDLDDYAADDDQYE
EKLKKETLAFFCVATYGDGEPTDNAARFYKWFTEENERDIKLQQLAYGVF
ALGNRQYEHFNKIGIVLDEELCKKGAKRLIEVGLGDDDQSIEDDFNAWKE
SLWSELDKLLKDEDDKSVATPYTAVIPEYRVVTHDPRFTTQKSMESNVAN
GNTTIDIHHPCRVDVAVQKELHTHESDRSCIHLEFDISRTGITYETGDHV
GVYAENHVEIVEEAGKLLGHSLDLVFSIHADKEDGSPLESAVPPPFPGPC
TLGTGLARYADLLNPPRKSALVALAAYATEPSEAEKLKHLTSPDGKDEYS
QWIVASQRSLLEVMAAFPSAKPPLGVFFAAIAPRLQPRYYSISSSPRLAP
SRVHVTSALVYGPTPTGRIHKGVCSTWMKNAVPAEKSHECSGAPIFIRAS
NFKLPSNPSTPIVMVGPGTGLAPFRGFLQERMALKEDGEELGSSLLFFGC
RNRQMDFIYEDELNNFVDQGVISELIMAFSREGAQKEYVQHKMMEKAAQV
WDLIKEEGYLYVCGDAKGMARDVHRTLHTIVQEQEGVSSSEAEAIVKKLQ
TEGRYLRDVW.

8. The fusion protein according to claim 1, wherein said polypeptide comprising the hydrophilic domain of a plant P450 cytochrome or said polypeptide comprising the hydrophilic domain of an NADPH P450 reductase is free of a transmembrane domain.

9. A nucleic acid coding for a fusion protein according to claim 1.

10. A vector, preferably an expression vector, comprising a nucleic acid according to claim 9.

11. A host cell comprising a nucleic acid according to claim 8.

12. The host cell according to claim 11, said host cell being a bacterial cell, preferably Escherichia coli.

13. A process for producing a fusion protein comprising culturing a host cell according to claim 11 under conditions suitable for expression of the fusion protein.

14. A process for bioconverting a substrate with a fusion protein according to claim 1, comprising the following steps:
introducing said substrate into a culture medium comprising a host cell,
incubating said culture medium for a time sufficient for bioconversion of said substrate by said fusion protein, and
optionally, recovering the metabolites resulting from bioconversion of said substrate.

15. A host cell comprising a vector according to claim 10.
